(19) United States
(12) Patent Application Publication
(10) Pub. No.: US 2009/0253799 A1
(43) Pub. Date: Oct. 8, 2009

(54) DIVALENT HYDRAZIDE COMPOUND CONJUGATES FOR INHIBITING CYSTIC FIBROSIS TRANSMEMBRANE CONDUCTANCE REGULATOR

(75) Inventors: Alan S. Verkman, San Francisco, CA (US); Nitin D. Sonawane, San Francisco, CA (US)

Correspondence Address: SEED INTELLECTUAL PROPERTY LAW GROUP PLLC, 701 FIFTH AVE, SUITE 5400, SEATTLE, WA, 98104, US

(73) Assignee: THE REGENTS OF THE UNIVERSITY OF CALIFORNIA, Oakland, CA (US)

(21) Appl. No.: 12/418,147

(22) Filed: Apr. 3, 2009

Related U.S. Application Data
(60) Provisional application No. 61/042,651, filed on Apr. 4, 2008.

(51) Int. Cl. A61K 31/17; C07C 335/00

(52) U.S. Cl. 514/586; 564/27

(57) ABSTRACT

Provided herein are divalent hydrazide-polyethylene glycol conjugates that inhibit the ion transport activity of a cystic fibrosis transmembrane conductance regulator (CFTR). The conjugates described herein are useful for treating diseases, disorders, and sequelae of diseases, disorders, and conditions that are associated with aberrantly increased CFTR activity, for example, secretory diarrhea.

### CROSS-REFERENCE TO RELATED APPLICATION

This application claims the benefit of U.S. Provisional Application No. 61/042,651 filed Apr. 4, 2008, which is incorporated herein by reference in its entirety.

### STATEMENT OF GOVERNMENT INTEREST

This invention was made with government support under grants DK72517, HL73854, EB00415, EY13574, DK35124 and DK43840 awarded by National Institutes of Health. The government has certain rights in this invention.

### BACKGROUND

1. Field

Therapeutics are needed for treating diseases and disorders related to aberrant cystic fibrosis transmembrane conductance regulator protein (CFTR), such as increased intestinal fluid secretion, secretory diarrhea, and polycystic kidney disease. Small molecule conjugates are described herein that are potent inhibitors of CFTR activity and that may be used for treating such diseases and disorders.

2. Description of the Related Art

The cystic fibrosis transmembrane conductance regulator protein (CFTR) is a cAMP-activated chloride (Cl⁻) channel expressed in epithelial cells in mammalian airways, intestine, pancreas and testis. CFTR is the chloride-channel responsible for cAMP-mediated Cl⁻ secretion. Hormones, such as a β-adrenergic agonist, or a toxin, such as cholera toxin, leads to an increase in cAMP, activation of cAMP-dependent protein kinase, and phosphorylation of the CFTR Cl⁻ channel, which causes the channel to open. An increase in cell Ca²⁺ can also activate different apical membrane channels. Phosphorylation by protein kinase C can either open or shut Cl⁻ channels in the apical membrane. CFTR is predominantly located in epithelia where it provides a pathway for the movement of Cl⁻ ions across the apical membrane and a key point at which to regulate the rate of transepithelial salt and water transport.

CFTR chloride channel function is associated with a wide spectrum of disease, including cystic fibrosis (CF) and with some forms of male infertility, polycystic kidney disease and secretory diarrhea. Cystic fibrosis is a hereditary lethal disease caused by mutations in CFTR (see, e.g., Quinton, Physiol. Rev. 79:S3-S22 (1999); Boucher, Eur. Respir. J. 23:146-58 (2004)). Observations in human patients with CF and mouse models of CF indicate the functional importance of CFTR in intestinal and pancreatic fluid transport, as well as in male fertility (Grubb et al., Physiol. Rev. 79:S193-S214 (1999); Wong, P. Y., Mol. Hum. Reprod. 4:107-110 (1997)). CFTR is expressed in enterocytes in the intestine and in cyst epithelium in polycystic kidney disease (see, e.g., O'Sullivan et al., Am. J. Kidney Dis. 32:976-983 (1998); Sullivan et al., Physiol. Rev. 78:1165-91 (1998); Strong et al., J. Clin. Invest. 93:347-54 (1994); Mall et al., Gastroenterology 126:32-41 (2004); Hanaoka et al., Am. J. Physiol. 270:C389-C399 (1996); Kunzelmann et al., Physiol. Rev. 82:245-289 (2002); Davidow et al., Kidney Int. 50:208-18 (1996); Li et al., Kidney Int. 66:1926-38 (2004); Al-Awqati, J. Clin. Invest. 110:1599-1601 (2002); Thiagarajah et al., Curr. Opin. Pharmacol. 3:594-99 (2003)).

High-affinity CFTR inhibitors have clinical applications in the therapy of secretory diarrheas. Cell culture and animal models indicate that intestinal chloride secretion in enterotoxin-mediated secretory diarrheas occurs mainly through the CFTR (see, e.g., Clarke et al., Science 257:1125-28 (1992); Gabriel et al., Science 266:107-109 (1994); Kunzelmann and Mall, Physiol. Rev. 82:245-89 (2002); Field, M. J. Clin. Invest. 111:931-43 (2003); and Thiagarajah et al., Gastroenterology 126:511-519 (2003)).

Diarrheal disease in children is a global health concern: approximately four billion cases among children occur annually, resulting in at least two million deaths. Travelers' diarrhea affects approximately 6 million people per year. Antibiotics are routinely used to treat diarrhea; however, the antibiotics are ineffective for treating many pathogens, and the use of these drugs contributes to development of antibiotic resistance in other pathogens.

Oral replacement of fluid loss is also routinely used to treat diarrhea, but is primarily palliative. Therapy directed at reducing intestinal fluid secretion (‘anti-secretory therapy’) has the potential to overcome limitations of existing therapies.

Several CFTR inhibitors have been discovered, although many exhibit weak potency and lack CFTR specificity. The oral hypoglycemic agent glibenclamide inhibits CFTR Cl⁻ conductance from the intracellular side by an open channel blocking mechanism (Sheppard & Robinson, J. Physiol., 503:333-346 (1997); Zhou et al., J. Gen. Physiol. 120:647-62 (2002)) at high micromolar concentrations where it affects other Cl⁻ and cation channels (Edwards & Weston, 1993; Rabe et al., Br. J. Pharmacol. 110: 1280-81 (1995); Schultz et al., Physiol. Rev. 79:S109-S144 (1999)). Other non-selective anion transport inhibitors including diphenylamine-2-carboxylate (DPC), 5-nitro-2(3-phenylpropyl-amino)benzoate (NPPB), and flufenamic acid also inhibit CFTR by occluding the pore at an intracellular site (Dawson et al., Physiol. Rev., 79:S47-S75 (1999); McCarty, J. Exp. Biol., 203:1947-62 (2000)).

A need exists for CFTR inhibitors, particularly those that are safe, non-absorbable, highly potent, inexpensive, and chemically stable.

### BRIEF SUMMARY

Briefly, provided herein are divalent hydrazide compound-polyethylene glycol (PEG) conjugates that are useful for treating diseases and disorders associated with aberrantly increased cystic fibrosis transmembrane conductance regulator (CFTR) chloride channel activity. In certain embodiments, two malonic hydrazide compounds are conjugated to a polymer moiety. In other embodiments, two glycine hydrazide compounds are conjugated to a polymer moiety. Embodiments provided herein include divalent polymer conjugate compounds useful as inhibitors of the cystic fibrosis transmembrane conductance regulator (CFTR) chloride channel and which have one of the following structures I or II:

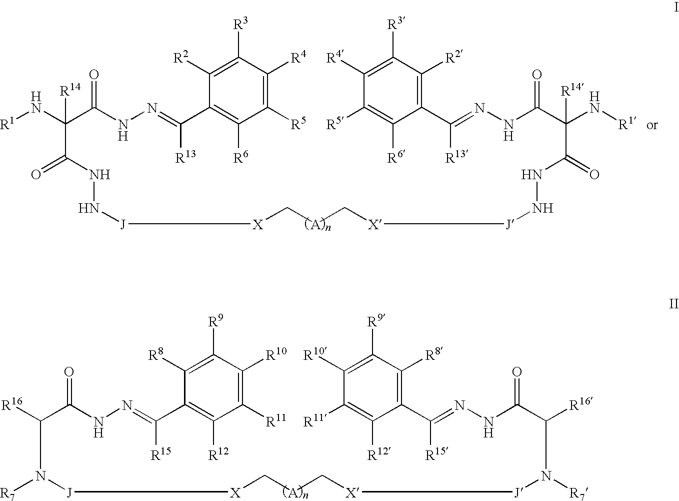

or a pharmaceutically acceptable salt, prodrug, or stereoisomer thereof, wherein each of X, X′, J, J′, n, R¹, R^{1′}, R², R^{2′}, R³, R^{3′}, R⁴, R^{4′}, R⁵, R^{5′}, R⁶, R^{6′}, R⁷, R^{7′}, R⁸, R^{8′}, R⁹, R^{9′}, R¹⁰, R^{10′}, R¹¹, R^{11′}, R¹², R^{12′}, R¹³, R^{13′}, R¹⁴R^{14′}, R¹⁵, R^{15′}, R¹⁶, and R^{16′} are as defined herein.

In certain embodiments, the polymer is polyethylene glycol (PEG) and two malonic hydrazide compounds are conjugated to a PEG moiety (i.e., A is —CH₂—O—CH₂—). In other embodiments, two glycine hydrazide compounds are conjugated to a PEG moiety (i.e., A is —CH₂—O—CH₂—). Embodiments provided herein include divalent PEG conjugate compounds useful as inhibitors of the cystic fibrosis transmembrane conductance regulator (CFTR) chloride channel and which have one of the following structures I(a) or II(a):

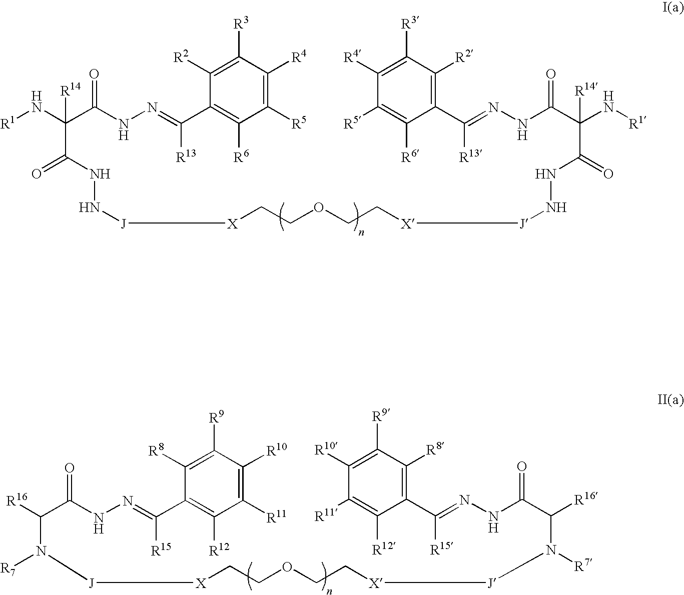

or a pharmaceutically acceptable salt, prodrug, or stereoisomer thereof, wherein each of X, X′, J, J′, n, R¹, R^{1′}, R², R^{2′}, R³, R^{3′}, R⁴, R^{4′}, R⁵, R^{5′}, R⁶, R^{6′}, R⁷, R^{7′}, R⁸, R^{8′}, R⁹, R^{9′}, R¹⁰, R^{10′}, R¹¹, R^{11′}, R¹², R^{12′}, R¹³, R^{13′}, R¹⁴, R^{14′}, R¹⁵, R^{15′}, R¹⁶, and R^{16′} are as defined herein. Also provided herein are substructures and divalent hydrazide PEG conjugate compounds of formulae and subformulae I(b), I(c), I(d), I(e), I(f), I(g), I(h), I(i), I(j), II(b), II(c), II(d), II(e), and II(f), and II((C)-(F)), as described in greater detail herein.

Also provided herein are methods of preparing divalent polymer conjugate compounds of structure I and II and of preparing divalent PEG conjugate compounds of structure I(a) and II(a) (and substructures thereof), pharmaceutical preparations of the same, and methods for inhibiting the cystic fibrosis transmembrane conductance regulator (CFTR) chloride channel, and for treating diseases, disorders, and conditions associated with aberrantly increased CFTR activity.

In another embodiment, a composition is provided wherein the composition comprises a pharmaceutically acceptable excipient and at least one divalent hydrazide polymer compound that has the structure of formula I or II. In another embodiment, a composition is provided wherein the composition comprises a pharmaceutically acceptable excipient and at least one divalent hydrazide-PEG conjugate compound that has a structure of formula I(a) or II(a) or substructures and structures of formulae I(b), I(c)-I(j), II(b), II(c), II(d), II(e), and II(f), II((C)-(F)) as described above and in greater detail herein.

In one embodiment, a method is provided method for treating a disease or disorder associated with aberrantly increased ion transport by cystic fibrosis transmembrane conductance regulator (CFTR), the method comprising administering to a subject the composition as described above and herein (which comprises a pharmaceutically acceptable excipient and at least one divalent hydrazide-polymer conjugate compound that has a structure of formula I or II). In another embodiment, a method is provided method for treating a disease or disorder associated with aberrantly increased ion transport by cystic fibrosis transmembrane conductance regulator (CFTR), the method comprising administering to a subject the composition as described above and herein (which comprises a pharmaceutically acceptable excipient and at least one divalent hydrazide-PEG conjugate compound that has a structure of formula I(a) or II(a) or substructures of formulae I(b), I(c)-I(j), II(b), II(c), II(d), I(e), and II(f), and II((C)-(F)), and other specific substructures and structures as described above and in greater detail herein), wherein ion transport by CFTR is inhibited. In a particular embodiment, the disease or disorder is aberrantly increased intestinal fluid secretion. In another particular embodiment, the disease or disorder is secretory diarrhea. In a certain embodiment, secretory diarrhea is caused by an enteric pathogen. In specific embodiments, the enteric pathogen is Vibrio cholerae, Clostridium difficile, Escherichia coli, Shigella, Salmonella, rotavirus, Giardia lamblia, Entamoeba histolytica, Campylobacter jejuni, and Cryptosporidium. In another certain embodiment, the secretory diarrhea is induced by an enterotoxin. In specific embodiments, the enterotoxin is a cholera toxin, a E. coli toxin, a Salmonella toxin, a Campylobacter toxin, or a Shigella toxin. In particular embodiments, secretory diarrhea is a sequelae of ulcerative colitis, irritable bowel syndrome (IBS), AIDS, chemotherapy, or an enteropathogenic infection. In specific embodiments, the subject is a human or non-human animal.

In another embodiment, a method is provided herein for inhibiting ion transport by a cystic fibrosis transmembrane conductance regulator (CFTR) comprising contacting (a) a cell that comprises CFTR and (b) at least one divalent hydrazide-polymer conjugate compound that has a structure of formula I or II. In another embodiment, a method is provided herein for inhibiting ion transport by a cystic fibrosis transmembrane conductance regulator (CFTR) comprising contacting (a) a cell that comprises CFTR and (b) at least one divalent hydrazide-PEG conjugate compound that has a structure of formula I(a) or II(a) or substructures of formulae I(b), I(c)-I(j), II(b), II(c), II(d), II(e), and II(f), and II((C)-(F)), and specific structures as described herein, under conditions and for a time sufficient for the CFTR and the compound to interact, thereby inhibiting ion transport by CFTR.

In yet another embodiment, a method is provided for treating secretory diarrhea comprising administering to a subject a pharmaceutically acceptable excipient and at least one divalent hydrazide-polymer conjugate compound that has a structure of formula I or II. In yet another embodiment, a method is provided for treating secretory diarrhea comprising administering to a subject a pharmaceutically acceptable excipient and at least one divalent hydrazide-PEG conjugate compound that has a structure of formula I(a) or II(a) or substructures of formulae I(b), I(c)-I(j), II(b), II(c), II(d), II(e), and II(f), and II((C)-(F)), and other specific structures described herein. In a specific embodiment, the subject is a human or non-human animal.

Also provided herein is use of any one of the divalent hydrazide-polymer conjugate compound, including at least one divalent hydrazide-PEG conjugate compound that has a structure of formula I(a) or II(a) or substructures of formulae I(b), I(c)-I(j), II(b), II(c), II(d), II(e), and II(f), and II((C)-(F)), and other specific structures described herein for preparation of a pharmaceutical composition for treating a disease or disorder associated with aberrantly increased CFTR activity, including aberrantly increased intestinal fluid secretion or secretory diarrhea.

As used herein and in the appended claims, the singular forms “a,” “and,” and “the” include plural referents unless the context clearly dictates otherwise. Thus, for example, reference to “a compound” or “a conjugate” includes a plurality of such compounds or conjugates, respectively. Similarly, reference to “a cell” or “the cell” includes reference to one or more cells and equivalents thereof (e.g., plurality of cells) known to those skilled in the art, and so forth. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

### BRIEF DESCRIPTION OF THE DRAWINGS

FIG. 1 depicts an exemplary synthesis of bisamino PEG of 40 kD molecular weight and bisamino PEG of 108 kDa. From left to right: TsCl, TEA, DCM; NaN₃, DMF, 40° C.; PPh₃, H₂O

FIGS. 2A-C depict NMR and mass spectra of monovalent MalH-PEG and divalent MalH-PEG-MalH conjugates. FIG. 2(A) depicts an ¹H-NMR spectrum of MalH-PEG20 kDa-MalH (MalH-PEG-MalH, 20 kDa), showing peaks corresponding to aliphatic and aromatic protons of PEG and MalH moieties, respectively. FIG. 2(B) shows negative ion electrospray ionization (ESI) mass spectra of monovalent conjugates, MalH-PEG750Da-OMe (MalH-PEG, 0.75 kDa) and MalH-PEG2 kDa-OMe (MalH-PEG, 2 kDa). FIG. 2(C) depicts a negative ion ESI mass spectra for divalent conjugate, MalH-PEG3 kDa-MalH (MalH-PEG-MalH, 3 dKa), showing the peaks for [M]³⁻ and [M]⁴⁻ ions with polydispersity.

FIGS. 3A-C depict CFTR inhibition by MalH-PEG and MalH-PEG-MalH conjugates. FIG. 3(A) shows original fluorescence assay data for CFTR inhibition by MalH-PEG20 kDa-MalH (MalH-PEG-MalH, 20 dKa) (left) and MalH-PEG20 kDa-OMe (MalH-PEG, 20 kDa) (right). CFTR was maximally stimulated by multiple agonists (forskolin, IBMX, and apigenin) in stably transfected FRT cells co-expressing human CFTR and the yellow fluorescent protein YFP-H148Q/I152L. The fluorescence decrease following iodide addition represents CFTR halide conductance. FIG. 3(B) shows concentration-inhibition data for indicated monovalent and divalent conjugates determined from the fluorescence assay (error bars represent Standard Error (S.E.), n=3-5). Data were fitted to a single site inhibition model. FIG. 3(C) illustrates fitted IC₅₀ values for monovalent and divalent conjugates as a function of molecular size, with calculated gyration radii shown (left). FIG. 3(C)(right) shows fitted Hill coefficients. At each molecular size, IC₅₀ values and Hill coefficients different significantly (p<0.01; Student's t test). Error bars represent ±S.E.

FIGS. 4A-C show the results from short-circuit current measurements of CFTR inhibition. In FRT cells expressing human wildtype CFTR, CFTR-mediated apical membrane chloride current was measured after permeabilization of the basolateral membrane in the presence of a chloride gradient (see Example 2). CFTR was activated by 20 μM forskolin and indicated concentrations of divalent MalH-PEG-MalH conjugates (PEG at 3, 10, 20, and 40 kDa), as shown in FIG. 4(A), and monovalent MalH-PEG conjugates (PEG at 2, 10, and 20 kDa), as shown in FIG. 4(B) were added to apical bathing solution. FIG. 4(C) shows the deduced IC₅₀ values for monovalent and divalent MalH conjugated to PEG at different molecular weights as shown (S.E., n=3-5).

FIGS. 5A-G illustrate an electrophysiological analysis of CFTR inhibition by the 20 kDa MalH-PEG conjugates. FIGS. 5(A) and 5(B) show representative whole-cell membrane currents from CFTR-expressing FRT cells. Each panel shows superimposed membrane currents induced at different membrane potentials (from −100 to +1000 mV) in 20 mV steps at 600 ms duration. Each pulse was followed by a 600 ms step of −100 mV. The interpulse interval was 4 s. Currents were measured before (upper panels), during (middle panels), and after (lower panels) application of the MalH-PEG conjugates (0.6 μM for MalH-PEG-MalH; 15 μM for MalH-PEG). Forskolin (5 μM) was present throughout all measurements. FIGS. 5(C) and 5(D) show current-voltage relationships from whole-cell experiments, which were measured as in 5(A) and 5(B). The current amplitude was reported as an average value at the end (550-600 ms) of the pulse, normalized to cell capacitance. Each point is the average. Error bars represent ±S.E., (4-5 experiments). FIG. 5(E) depicts the kinetics of current relaxations elicited at indicated membrane voltages. Single exponential regressions are shown. FIG. 5(F) shows time constants for block and unblock measured at the indicated membrane voltages (Vm) by single exponential regression of current relaxations. Closed circles denote monovalent MalH-PEG; open circles denote divalent MalH-PEG-MalH. Error bars represent ±S.E., (4-5 experiments; *p<0.05). Concentrations were 0.6 μM for MalH-PEG-MalH and 15 μM for MalH-PEG. FIG. 5G illustrates the effect of extracellular Cl⁻ concentration on MalH-PEG-MalH block. Inhibition of CFTR current measured at 60 mV in the presence of 154 or 20 mM extracellular Cl⁻. Symbols are the mean of three to five different experiments. Error bars represent ±S.E. (*p<0.05).

FIGS. 6A-D depict outside-out patch-clamp recordings of CFTR inhibition by MalH-PEG conjugates. FIGS. 6(A) and 6(B) illustrate representative traces at 60 mV showing CFTR single channel activity in the absence and presence of 2 μM divalent 20 kDa MalH-PEG-MalH and 15 μM monovalent 20 kDa MalH-PEG conjugates, respectively. Pipette (intracellular) solution contained 1 mM ATP and 5 μg/ml protein kinase A catalytic subunit. Channel openings are shown as upward deflections from the closed channel level (lowest currents) (indicated by short lines on the right side of traces). FIGS. 6(C) and 6(D) summarize the results of a single channel analysis for divalent and monovalent malonic hydrazide-PEG 20 kDa conjugates, respectively. Error bars represent one S.E., (4 experiments, *, p<0.05; **, p<0.01).

FIGS. 7A-C show the antidiarrheal efficacy of divalent MalH-PEG conjugates in both in vitro and in vivo models. FIG. 7(A) demonstrates inhibition of CFTR stimulated short-circuit current in human intestinal T84 cells (non-permeabilized) by MalH-PEG20 kDa-MalH (MalH-PEG-MalH, 20 kDa) and MalH-PEG40 kDa-MalH (MalH-PEG-MalH, 40 kDa). Amiloride was added prior to forskolin. Data are representative of three sets of experiments. Where indicated, forskolin (forsk) (20 μM) was added to activate CFTR. Baseline current was 3-7 μA. FIG. 7(B) shows intestinal fluid accumulation at 6 h, quantified by intestinal loop weight-to-length ration, in closed mid-jejunal loops in mice (error bars indicate one S.E., 6-8 loops studied per condition, * P<0.05, ANOVA). FIG. 7(C) demonstrates improved survival of suckling mice (32 mice per group) following gavage with cholera toxin without versus with MalH-PEG20 kDa-MalH (500 μmol, left) and MalH-PEG40 kDa-MalH (500 μmol, right). The ‘vehicle control’ mice were identically processed but did not receive cholera toxin or inhibitors.

### DETAILED DESCRIPTION

Significantly improved hydrazide compound conjugates that inhibit CFTR activity are described herein. Two hydrazide compounds, for example two malonic hydrazide compounds, are covalently attached (i.e., conjugated, reacted with, or joined together in a manner to form a covalent bond) to a polymer with two reactive functional groups (including but not limited to polyethylene glycol (PEG)) to provide a divalent hydrazide-polymer conjugate compound (for example, a divalent hydrazide PEG-conjugate compound). The exemplary divalent malonic hydrazide-PEG conjugate compounds described herein have significantly improved potency (approximately 10-20 fold improvement) compared with monovalent malonic hydrazide-PEG conjugate compounds. The divalent malonic hydrazide PEG conjugate compounds are minimally absorbable by cells and, thus, minimize potential cellular and systemic toxicity.

Specific inhibitors of CFTR activity useful for altering intestinal fluid secretion include the non-absorbable glycine hydrazide compounds and malonic hydrazide compounds (see, e.g., Muanprasat et al., J. Gen. Physiol. 124:125-37 (2004); Sonawane et al., FASEB J. 20:130-32 (2006); U.S. Pat. No. 7,414,037; U.S. Patent Application Publication No. 2005/0239740; see also, e.g., Salinas et al., FASEB J. 19:431-33 (2005); Thiagarajah et al., FASEB J. 18:875-77 (2004))). Effective glycine hydrazide and malonic hydrazide inhibitors had an IC₅₀ of approximately 5 μM. However, binding of compounds with micromolar IC₅₀ to CFTR expressed in intestinal lumen may be reversed, particularly by washout of the compound from the intestine by rapid intestinal fluid transit in a subject affected with secretory diarrhea.

The divalent hydrazide-PEG conjugate compounds described herein, including divalent malonic hydrazide-PEG conjugate compounds, may therefore be used for treating diseases and disorders associated with aberrantly increased CFTR-mediated transepithelial fluid secretion. Such diseases and disorders include secretory diarrhea, which may be caused by enteropathogenic organisms including bacteria, viruses, and parasites, such as but not limited to Vibrio cholerae, Clostridium difficile, Escherichia coli, Shigella, Salmonella, rotavirus, Campylobacter jejuni, Giardia lamblia, Entamoeba histolytica, Cyclospora, and Cryptosporidium or by toxins such as cholera toxin and Shigella toxin. The conjugates described herein may also be useful for treating secretory diarrhea that is a sequelae of a disease, disorder, or condition, including but not limited to AIDS, administration of AIDS related therapies, chemotherapy, and inflammatory gastrointestinal disorders such as ulcerative colitis, inflammatory bowel disease (IBD), and Crohn's disease.

Small molecule inhibitors of the cystic fibrosis transmembrane conductance regulator protein (CFTR), which is a cAMP-activated chloride (Cl⁻) channel, include glycine hydrazide, oxamic hydrazide, and malonic hydrazide compounds (see, e.g., U.S. Pat. No. 7,414,037; U.S. Patent Application Publication No. 2005/0239740; see also, e.g., Salinas et al., FASEB J. 19:431-33 (2005); Thiagarajah et al., FASEB J. 18:875-77 (2004)). Any one of these compounds may be conjugated to (i.e., linked, attached, joined, covalently bonded to) polyethylene glycol that is capable of binding to (i.e., associating by ionic interaction (coulombic forces), hydrophobic, hydrophilic, lipophilic interaction, hydrogen bonding, or any combination thereof, to) a cell that expresses CFTR. Without wishing to be bound by theory, these minimally absorbable divalent hydrazide-PEG conjugate compounds may have increased potency compared with a non-conjugated compound, in part, because the conjugated compounds are not washed away from the intestinal lumen.

Monovalent polyethylene glycol (PEG) conjugates of malonic acid hydrazide (MalH) analogs block CFTR chloride current rapidly and fully when added to solutions bathing the external cell surface (see, e.g., Sonawane, et al., FASEB J. 20:130-132 (2006)). Monovalent MalH-PEG conjugates prevent cholera toxin-induced intestinal fluid secretion when present in the lumen of closed intestinal loops in mice. The IC₅₀ values for CFTR inhibition by monovalent MalH-PEG conjugate compounds are generally >5 μM, however, and inhibition is reversed rapidly following washout. Therefore, in subjects who have severe secretory diarrhea, rapid intestinal fluid transit may significantly reduce the therapeutic effect by dilutional washout of the compound. Unexpectedly, divalent MalH-PEG conjugates had significantly improved potency (10-20 fold) compared with monovalent MalH-PEG conjugates.

### Divalent Hydrazide Polymer Conjugate Compounds

Provided herein are divalent hydrazide-polymer conjugate compounds that are inhibitors of the cystic fibrosis transmembrane conductance regulator (CFTR) chloride channel. In one embodiment, a polymer is joined at each of two reactive termini (also called herein terminal ends) to a malonic hydrazide or glycine hydrazide compound moiety to provide a divalent hydrazide structure: hydrazide-polymer-hydrazide conjugate compound. In general, a polymer as described herein is comprised of repeating units, which may be depicted as (A)ₙ, in which A is the repeating unit and n is an integer between 0 and 2500. A suitable polymer that may be used for making a divalent hydrazide polymer conjugate compound has two nucleophilic terminal groups (e.g., an oxygen, nitrogen, or sulfur containing group) that may be joined to a linker group (e.g., X and X′ described in detail herein), which linker group may be joined to a spacer group (e.g., J and J′, respectively, as described in detail herein). Spacer J is joined to one hydrazide compound moiety and J′ is attached to a second hydrazide compound moiety. Exemplary polymers include, but are not limited to, polymers such as polyethylene glycol (PEG), polypropylene glycol, polyhydroxyethyl glycerol and other polyoxyalkyl polyethers. Another suitable polymer is polyethylene amine, an amine analog of PEG, which has a subunit of (—CH₂NH—CH₂—). Other polymers include polyethylenimines (PEI), dendrimers, and carbohydrates (such as dextrans), for which reactive groups can be limited to two, such that each of the two reactive groups can be joined to each of two hydrazide compounds to provide a dimer hydrazide-polymer conjugate.

An embodiment provided herein is a divalent malonic hydrazide-polymer conjugate compound that has the following structure I:

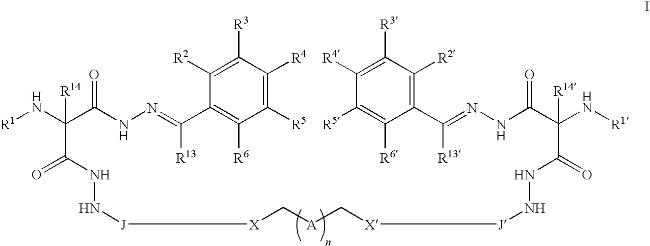

or a pharmaceutically acceptable salt, prodrug, or stereoisomer thereof wherein:

R¹ and R^{1′} are the same or different and independently optionally substituted phenyl, optionally substituted heteroaryl, optionally substituted quinolinyl, optionally substituted anthracenyl, or optionally substituted naphthalenyl;

R², R^{2′}, R³, R^{3′}, R⁴, R^{4′}, R⁵, R^{5′}, R⁶, and R^{6′} are each the same or different and independently hydrogen, hydroxy, C₁₋₈ alkyl, C₁₋₈ alkoxy, carboxy, halo, nitro, cyano, —SO₃H, —S(═O)₂NH₂, aryl, and heteroaryl;

R¹³, R^{13′}, R¹⁴, and R^{14′} are each the same or different and independently hydrogen or C₁₋₈ alkyl;

X and X′ are each the same or different linker moiety;

J and J′ are each the same or different spacer moiety;

A is a subunit of a polymer; and

n is an integer between 0 and 2,500.

In certain embodiments, n is any integer between 0 and 10, between 0 and 100, between 1 and 5, between 1 and 10, between 1 and 100, between 1 and 550, between 1 and 1000, between 10 and 2500, between 10 and 2000, between 50 and 1000, between 250 and 1000, or between 450 and 1000. In more specific embodiments of structures I, n is any integer between 50 and 1000. In another specific embodiment, n is any integer between 200 and 300. In yet another specific embodiment, n is any integer between 450 and 550. In still another specific embodiment, n is any integer between 900 and 1000. In another specific embodiment, n is 0.

In certain embodiments, A is a subunit of the polymer polyethylene glycol (PEG) (i.e., —CH₂—O—CH₂—). In another embodiment, A is a subunit of a polymer selected from a polyethylenimine (PEI), a dendrimer, or a carbohydrate (such as a dextran), wherein the polymer has two termini (i.e., terminal ends) one of which is joined to linker X and the other (or second) of which is joined to the linker X′. In other embodiments, A is an amino acid and the polymer is a peptide or polypeptide. In certain specific embodiments, when A is an amino acid, n is between 1 and 5, 1 and 10, 1 and 15, 1 and 20, 1 and 40, 1 and 50, between 1 and 100, or between 100 and 500.

In another specific embodiment, A is —CH₂—NH—CH₂— (a monomer of polyethylene amine). In certain specific embodiments, n is an integer between 1 and 5, 1 and 10, 1 and 20, 1 and 30, between 1 and 100, between 100 and 500, or between 500 and 1000.

In another embodiment, A is optionally substituted alkanediyl, optionally substituted alkenylene (divalent aliphatic hydrocarbon containing at least one double bond), or optionally substituted alkynylene (divalent aliphatic hydrocarbon containing at least one triple bond). In certain specific embodiments, when A is an alkanediyl, alkenylene, or alkynylene n is an integer between 2 and 5, 2 and 10, 2 and 20, or between 2 and 30, or between 2 and 50.

In still other embodiments, A is optionally substituted aryl or optionally substituted cycloalkyl. In specific embodiments, A is optionally substituted phenyl, and in other specific embodiments, A is optionally substitute cyclohexyl. In certain specific embodiments, when A is an aryl or cycloalkyl, n is an integer between 1 and 3, 1 and 5, 1 and 10, 1 and 20, or 1 and 30, or between 1 and 100.

In yet another embodiment, n is 0 and A is absent.

In certain embodiments, each of R¹ R^{1′}, R², R^{2′}, R³, R^{3′}, R⁴, R^{4′}, R⁵, R^{5′}, R⁶, and R^{6′}, R¹³, R^{13′}, R¹⁴, R^{14′}, X, X′, J, and J′ are as defined herein (see below with respect to divalent hydrazide-PEG conjugate compounds).

### Divalent Hydrazide-PEG Conjugate Compounds

In one embodiment, A is —CH₂—O—CH₂— and the polymer is polyethylene glycol (PEG). Provided herein are divalent hydrazide-PEG conjugate compounds that are inhibitors of the cystic fibrosis transmembrane conductance regulator (CFTR) chloride channel. An embodiment provided herein is a divalent malonic hydrazide-PEG conjugate compound, which has the following structure I(a):

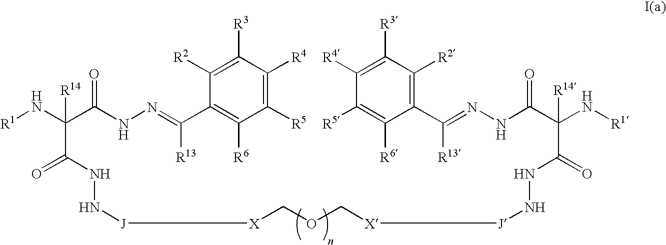

or a pharmaceutically acceptable salt, prodrug, or stereoisomer thereof wherein:

R¹ and R^{1′} are the same or different and independently optionally substituted phenyl, optionally substituted heteroaryl, optionally substituted quinolinyl, optionally substituted anthracenyl, or optionally substituted naphthalenyl;

R², R^{2′}, R³, R^{3′}, R⁴, R^{4′}, R⁵, R^{5′}, R⁶, and R^{6′} are each the same or different and independently hydrogen, hydroxy, C₁₋₈ alkyl, C₁₋₈ alkoxy, carboxy, halo, nitro, cyano, —SO₃H, —S(═O)₂NH₂, aryl, and heteroaryl;

R¹³, R^{13′}, R¹⁴, and R^{14′} are each the same or different and independently hydrogen or C₁₋₈ alkyl;

X and X′ are each the same or different linker moiety;

J and J′ are each the same or different spacer moiety; and

n is an integer between 0 and 2,500.

In certain embodiments of structures I(a), n is any integer between 0 and 10, between 0 and 100, between 1 and 5, between 1 and 10, between 1 and 100, between 1 and 300, between 1 and 550, between 1 and 1000, between 1 and 2500, between 10 and 2500, between 10 and 2000, between 50 and 1000, between 250 and 1000, or between 450 and 1000. In more specific embodiments of structures I(a), n is any integer between 50 and 1000. In another specific embodiment, n is any integer between 200 and 300. In yet another specific embodiment, n is any integer between 450 and 550. In still another specific embodiment, n is any integer between 900 and 1000. In another specific embodiment, n is 0.

In certain embodiments, R¹³, R^{13′}, R¹⁴, and R^{14′} are the same or different and independently hydrogen or methyl. In a more specific embodiment, each of R¹³, R^{13′}, R¹⁴, and R^{14′} is hydrogen.

In more specific embodiments of structure I and structure I(a), R¹ and R^{1′} are the same or different and independently 1-naphthalenyl or 2-naphthalenyl, optionally substituted with one or more of halo, hydroxy, —SH, —SO₃H, C₁₋₈ alkyl, and C₁₋₈ alkoxy; aryloxy; mono-halophenyl; di-halophenyl; mono-alkylphenyl; 2-anthracenyl; or 6-quinolinyl. In a specific embodiment, halo is chloro. In other specific embodiments of structure I and structure I(a), R¹ and R^{1′} are the same or different and independently unsubstituted phenyl, or substituted phenyl wherein phenyl is substituted with one or more of hydroxy, C₁₋₈ alkyl, aryl, aryloxy, —SO₃H, C₁₋₈ alkoxy, or halo wherein halo is fluoro, chloro, bromo, or iodo. In a specific embodiment, halo is chloro. In another specific embodiment, C₁₋₈ alkyl is methyl. In yet another specific embodiment, R¹ and R^{1′} are the same or different and independently phenyl substituted with methyl or chloro. In other specific embodiments, R¹ and R^{1′} are the same or different and independently quinolinyl or anthracenyl, optionally substituted with one or more of halo, hydroxy, C₁₋₈ alkyl, or C₁₋₈ alkoxy.

In other specific embodiments of structure I and I(a), R¹ and R^{1′} are the same or different and independently 2-halophenyl; 4-halophenyl; -2-4-halophenyl, 4-methylphenyl; mono-(halo)naphthalenyl, di-(halo)naphthalenyl, tri-(halo)naphthalenyl, mono-(hydroxy)naphthalenyl, di-(hydroxy)naphthalenyl, tri-(hydroxy)naphthalenyl, mono-(alkoxy)naphthalenyl, di-(alkoxy)naphthalenyl, tri-(alkoxy)naphthalenyl, mono-(aryloxy)naphthalenyl, di-(aryloxy)naphthalenyl, mono-(alkyl)naphthalenyl, di-(alkyl)naphthalenyl, tri-(alkyl)naphthalenyl, mono-(hydroxy)-naphthalene-sulfonic acid, mono-(hydroxy)-naphthalene-disulfonic acid, mono(halo)-mono(hydroxy)naphthalenyl; di(halo)-mono(hydroxy)naphthalenyl; mono(halo)-di(hydroxy)naphthalenyl; di(halo)-di(hydroxy)naphthalenyl; mono-(alkyl)-mono-(alkoxy)-naphthalenyl, mono-(alkyl)-di-(alkoxy)-naphthalenyl, mono-(halo)phenyl, di-(halo)phenyl, tri-(halo) phenyl, mono-(hydroxy)phenyl, di-(hydroxy)phenyl, tri-(hydroxy)phenyl, mono-(alkoxy)phenyl, di-(alkoxy)phenyl, tri-(alkoxy)phenyl, mono-(aryloxy)phenyl, di-(aryloxy)phenyl, mono-(alkyl)phenyl, di-(alkyl)phenyl, tri-(alkyl)phenyl, mono-(hydroxy)-phenyl-sulfonic acid, mono-(hydroxy)-phenyl-disulfonic acid, mono(halo)-mono(hydroxy)phenyl, di(halo)-mono(hydroxy)phenyl, mono(halo)-di(hydroxy)phenyl, di(halo)-di(hydroxy)phenyl, mono-(alkyl)-mono-(alkoxy)-phenyl, or mono-(alkyl)-di-(alkoxy)-phenyl wherein halo is fluoro, chloro, bromo, or iodo. In a particular embodiment, halo is chloro.

In even more specific embodiments of structure I and structure I(a), R¹ and R^{1′} are the same or different and independently 2-naphthalenyl, 2-chlorophenyl, 4-chlorophenyl, 2-4-dichlorophenyl, 4-methylphenyl, 2-anthracenyl, or 6-quinolynyl. In other specific embodiments, of structure I and structure I(a), R¹ and R^{1′} are each the same or different and independently 2-naphthalenyl or 4-chlorophenyl.

In other specific embodiments of structure I and structure I(a) described above, R², R^{2′}, R³, R^{3′}, R⁴, R^{4′}, R⁵, R^{5′}, R⁶, and R^{6′} are each the same or different and independently hydrogen, hydroxy, halo, C₁₋₈ alkyl, C₁₋₈ alkoxy, or carboxy.

In other certain embodiments of structure I and structure I(a), R², R³, R⁴, R⁵, and R⁶, are each the same or different and independently selected from hydrogen, hydroxy, halo, C₁₋₈ alkyl, C₁₋₈ alkoxy, or carboxy, such that the phenyl group to which R², R³, R⁴, R⁵, and R⁶ are attached is substituted with one, two, or three halo; one or two carboxy; one, two, or three hydroxy; one or two halo and one, two, or three hydroxy; one or two halo, one or two hydroxy, and one C₁₋₈ alkoxy; one or two halo, one hydroxy, and one or two C₁₋₈ alkoxy; or one halo, one or two hydroxy, and one or two C₁₋₈ alkoxy, wherein halo is bromo, chloro, iodo, or fluoro; in a more specific embodiment, halo is bromo. In other specific embodiments, alkoxy is methoxy.

In other certain embodiments of structure I and structure I(a), R^{2′}, R^{3′}, R^{4′}, R^{5′}, and R^{6′} are each the same or different and independently selected from hydrogen, hydroxy, halo, C₁₋₈ alkyl, C₁₋₈ alkoxy, or carboxy, such that the phenyl group to which R², R^{3′}, R^{4′}, R^{5′}, and R^{6′} are attached is substituted with one, two, or three halo; one or two carboxy; one, two, or three hydroxy; one or two halo and one, two, or three hydroxy; one or two halo, one or two hydroxy, and one C₁₋₈ alkoxy; one or two halo, one hydroxy, and one or two C₁₋₈ alkoxy; or one halo, one or two hydroxy, and one or two C₁₋₈ alkoxy, wherein halo is bromo, chloro, iodo, or fluoro. In a more specific embodiment, halo is bromo. In other specific embodiments, alkoxy is methoxy.

In certain specific embodiments of structure I and structure I(a), R², R³, R⁴, R⁵, and R⁶ are the same or different and independently selected from hydrogen, hydroxy, halo, C₁₋₈ alkyl, C₁₋₈ alkoxy, or carboxy, such that the phenyl group to which R², R³, R⁴, R⁵ and R⁶ are attached is substituted with di(hydroxy); mono-(halo)-mono-(hydroxy); mono-(halo)-di-(hydroxy); mono-(halo)-tri-(hydroxy); di(halo)-mono-(hydroxy); di(halo)-di-(hydroxy); di(halo)-tri-(hydroxy); mono-(halo)-mono-(hydroxy)-mono-(alkoxy); mono-(halo)-di-(hydroxy)-mono-(alkoxy); mono-(halo)-mono-(hydroxy)-di-(alkoxy); mono-(halo)-di-(hydroxy)-di-(alkoxy); di-(halo)-mono-(hydroxy)-mono-(alkoxy); di-(halo)-di-(hydroxy)-mono-(alkoxy); or di-(halo)-mono-(hydroxy)-di-(alkoxy). In a specific embodiment, halo is bromo.

In certain specific embodiments of structure I and structure I(a), R^{2′}, R^{3′}, R^{4′}, R^{5′}, and R^{6′}, are the same or different and independently selected from hydrogen, hydroxy, halo, C₁₋₈ alkyl, C₁₋₈ alkoxy, or carboxy, such that the phenyl group to which R^{2′}, R^{3′}, R^{4′}, R^{5′}, R^{5′}, and R^{6′} is attached is substituted with di(hydroxy); mono-(halo)-mono-(hydroxy); mono-(halo)-di-(hydroxy); mono-(halo)-tri-(hydroxy); di(halo)-mono-(hydroxy); di(halo)-di-(hydroxy); di(halo)-tri-(hydroxy); mono-(halo)-mono-(hydroxy)-mono-(alkoxy); mono-(halo)-di-(hydroxy)-mono-(alkoxy); mono-(halo)-mono-(hydroxy)-di-(alkoxy); mono-(halo)-di-(hydroxy)-di-(alkoxy); di-(halo)-mono-(hydroxy)-mono-(alkoxy); di-(halo)-di-(hydroxy)-mono-(alkoxy); or di-(halo)-mono-(hydroxy)-di-(alkoxy). In a specific embodiment, halo is bromo.

In other certain specific embodiments of structure I and structure I(a), R², R³, R⁴, R⁵, and R⁶ are the same or different and independently selected from hydrogen, hydroxy, halo, C₁₋₈ alkyl, C₁₋₈ alkoxy, or carboxy, such that the phenyl group to which R², R³, R⁴, R⁵, and R⁶ is attached is 2-, 3-, or 4-halophenyl; 3,5-dihalophenyl; 2-, 3-, or 4-hydroxyphenyl; 2,4-dihydroxyphenyl; 3,5-dihalo-2,4,6-trihydroxyphenyl, 3,5-dihalo-2,4-dihydroxyphenyl; 3,5-dihalo-4-hydroxyphenyl; 3-halo-4-hydroxyphenyl; 3,5-dihalo-2-hydroxy-4-methoxyphenyl; or 4-carboxyphenyl, wherein halo is bromo, chloro, fluoro, or iodo. In another specific embodiment, halo is bromo.

In other certain specific embodiments of structure I and structure I(a), R^{2′}, R^{3′}, R^{4′}, R^{5′}, and R^{6′} are the same or different and independently selected from hydrogen, hydroxy, halo, C₁₋₈ alkyl, C₁₋₈ alkoxy, or carboxy, such that the phenyl group to which R^{2′}, R^{3′}, R^{4′}, R^{5′}, and R^{6′} is attached is 2-, 3-, or 4-halophenyl; 3,5-dihalophenyl; 2-, 3-, or 4-hydroxyphenyl; 2,4-dihydroxyphenyl; 3,5-dihalo-2,4,6-trihydroxyphenyl, 3,5-dihalo-2,4-dihydroxyphenyl; 3,5-dihalo-4-hydroxyphenyl; 3-halo-4-hydroxyphenyl; 3,5-dihalo-2-hydroxy-4-methoxyphenyl; or 4-carboxyphenyl, wherein halo is bromo, chloro, fluoro, or iodo. In another specific embodiment, halo is bromo.

In other certain specific embodiments of structure I and structure I(a), each of R³ and R⁵ is halo and each of R⁴ and R⁶ is hydroxy. In another specific embodiment, each of R³ and R⁵ is halo and R⁴ is hydroxy. In yet another specific embodiment, each of R³ and R⁵ is bromo and each of R⁴ and R⁶ is hydroxy. In still another specific embodiment, each of R³ and R⁵ is bromo, R⁴ is hydroxy, and R⁶ is hydrogen. In certain specific embodiments of structure I and structure I(a), each of R^{3′} and R^{5′} is halo and each of R^{4′} and R^{6′} is hydroxy. In another specific embodiment, each of R^{3′} and R^{5′} is halo and R^{4′} is hydroxy. In still another specific embodiment, each of R^{3′} and R^{5′} is bromo and each of R^{4′} and R^{6′} is hydroxy. In yet another specific embodiment, each of R^{3′} and R^{5′} is bromo, R^{4′} is hydroxy, and R^{6′} is hydrogen. In specific embodiments, each of R² and R^{2′} is hydrogen.

In certain specific embodiments of structure I and structure I(a), each of R³, R^{3′}, R⁵ and R^{5′} is halo and each of R⁴, R^{4′}, R⁶, and R^{6′} is hydroxy. In other certain specific embodiments, each of R² and R^{2′} is hydrogen.

In other specific embodiments of structure I and structure I(a), each of R³, R^{3′}, R⁵, and R^{5′} is halo and each of R⁴ and R^{4′} is hydroxy. In specific embodiments, each of R² and R^{2′} is hydrogen.

In yet more specific embodiments of structure I and structure I(a), each of R³, R^{3′}, R⁵, and R^{5′} is bromo, and each of R⁴, R^{4′}, R⁶, and R^{6′} is hydroxy. In specific embodiments, each of R² and R^{2′} is hydrogen.

In yet more specific embodiments of structure I and structure I(a), each of R³, R^{3′}, R⁵, and R^{5′} is bromo, each of R⁴ and R^{4′} is hydroxy, and each of R⁶ and R^{6′} is hydrogen. In specific embodiments, each of R² and R^{2′} is hydrogen.

In other more specific embodiments of structure I and structure I(a), R¹³, R^{13′}, R¹⁴, and R^{14′} are the same or different and independently hydrogen or methyl. In such embodiments, R¹ and R^{1′} are each the same or different and independently phenyl substituted with at least one chloro or methyl; 1-naphthalenyl; 2-naphthalenyl; 6-quinolinyl; or 2-anthracenyl. In specific embodiments, R², R³, R⁴, R⁵, R⁶, R^{2′}, R^{3′}, R^{4′}, R^{5′}, and R^{6′} are each the same or different and independently hydrogen, halo, methoxy, hydroxyl, or carboxy; in specific embodiments, halo is bromo. In certain specific embodiments, when each of R³, R⁴, R⁵, R⁶, R^{3′}, R^{4′}, R^{5′}, and R^{6′} is not hydrogen, R² and R^{2′} are each hydrogen.

The linker moieties X and X′ are each a functional group that may be used for conjugating the spacer J and spacer J′, respectively, to polyethylene glycol (i.e., (—CH₂—O—CH₂—)n) for the compounds having structure I(a) or to the polymer (A)ₙ for compounds having structure I. In certain embodiments of the compounds having structure I or I(a) as described above, the linker X and the linker X′ are the same or different and independently —NH—, —O—, or —S—. In a particular embodiment, X and X′ are the same and each is —NH—.

The spacer J and the spacer J′ are each independently a moiety that is a spacer between the polyethylene glycol moiety and each of two hydrazide compound moieties (which spacers are respectively conjugated to PEG via the linker X and X′), respectively, as set forth in the structure of formula I(a). Similarly, the spacer J and the spacer J′ are each independently a moiety that is a spacer between the polymer (A)ₙ and each of two hydrazide compound moieties (which spacers are respectively conjugated to the polymer via the linker X and X′), respectively, as set forth in the structure of formula I. Exemplary spacer moieties (i.e., -J- and -J′-) of the compounds having structure I or I(a) as described above include the following structures J1 through J29.

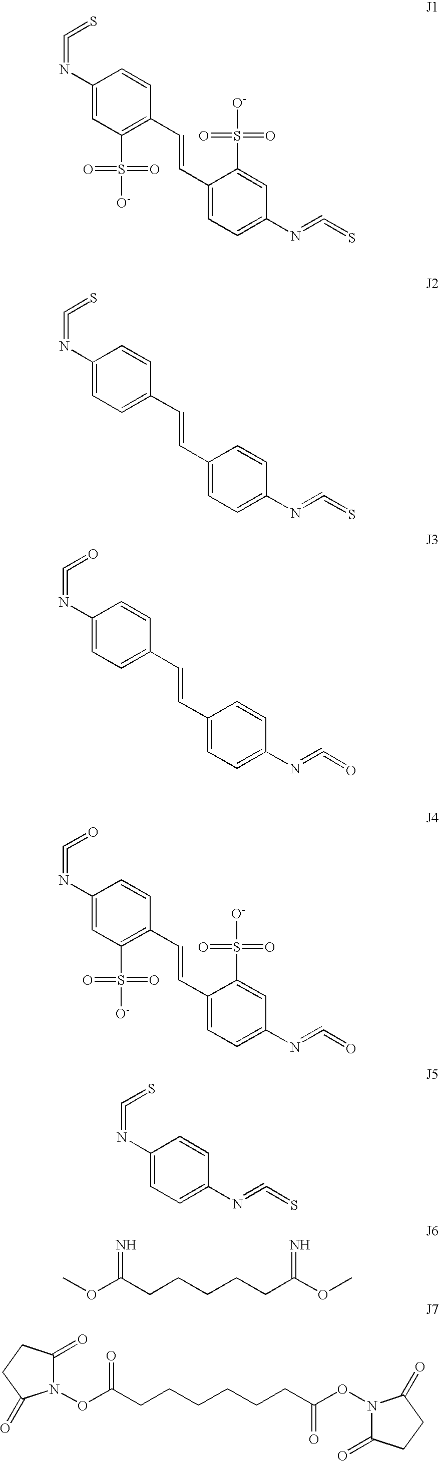

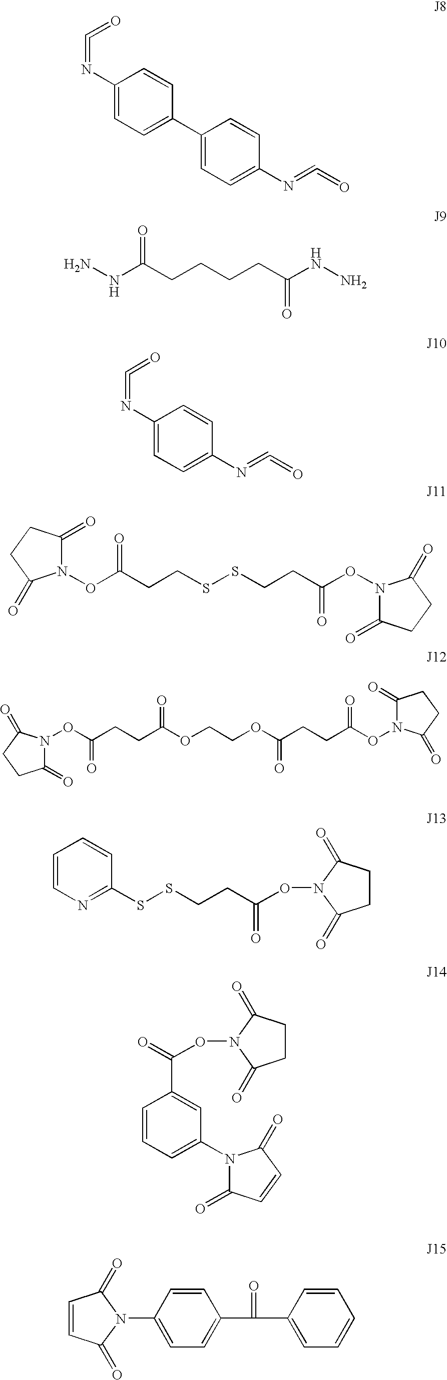

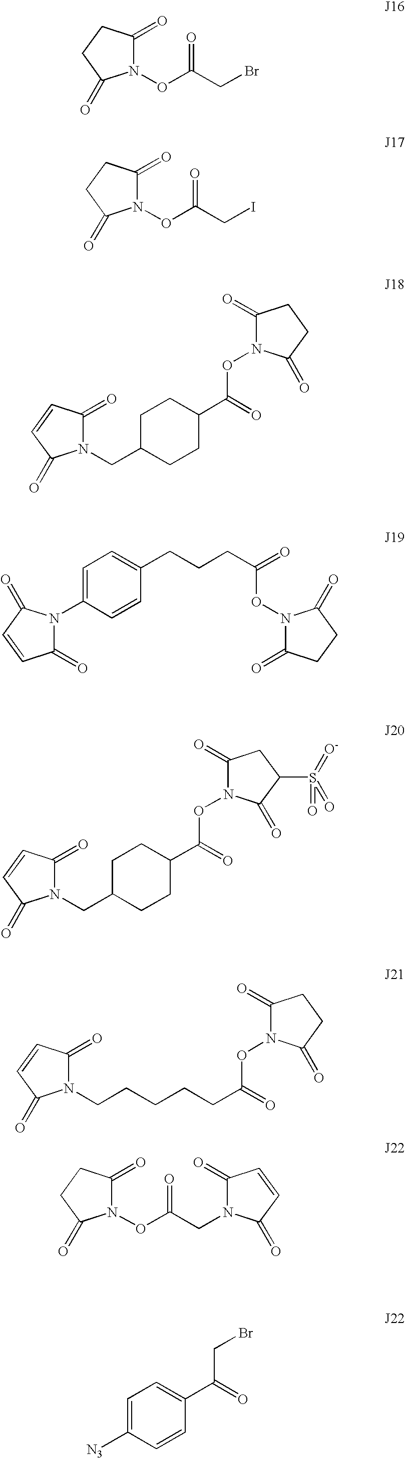

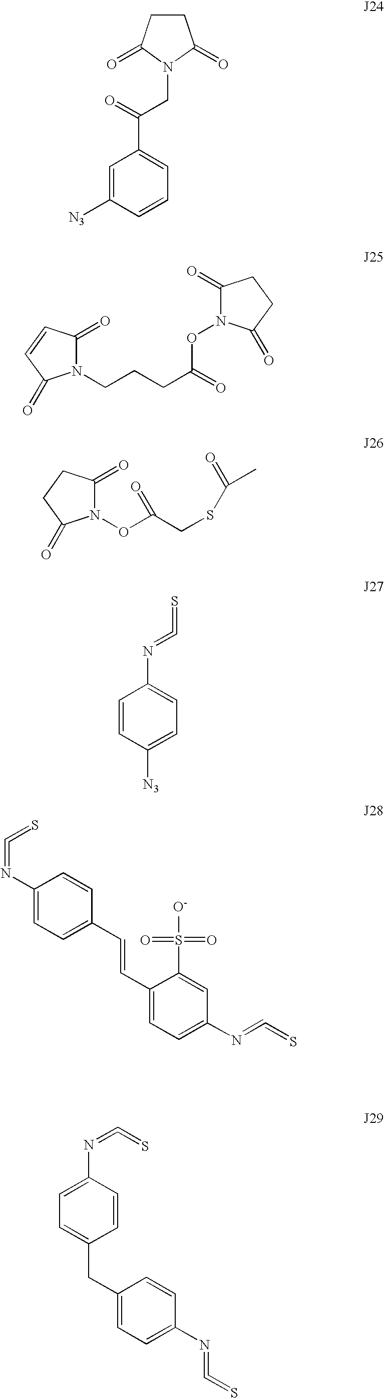

Each spacer J and J′ may be the same or different and selected from J1-J29. In certain embodiments, each of J and J′ are the same and each is J1 (4,4′-diisothiocyanostilbene-2,2′-disulfonic acid (DIDS)).

The exemplary structures shown above provide the chemical moiety that may be used as spacer J or spacer J′. As will be readily apparent to a person skilled in the chemical art, the structure of the spacer, such as any one of J1-J29, shown above and herein, will not be identical when the spacer is joined to the hydrazide moiety and to the linker moiety; that is, the above structures J 1-J29 represent a precursor structure of the spacer moieties or in certain instances, represent the reactant chemical moiety. The exemplary spacer moieties J1-J29 above, and other spacer moieties available in the art, have at least two reactive groups (i.e., functional groups), one of which is joined to one of the two hydrazide compounds of the dimer conjugate, and the other (or second) reactive group of the spacer is joined to the linker X (or to the linker X′). As used herein, an “end” of the spacer J and spacer J′ denotes each reactive group (i.e., functional group).

Each spacer J and spacer J′ has a first end and a second end, wherein the first end of spacer J is attached or joined to the hydrazide nitrogen atom of one hydrazide compound moiety as depicted in formulae I or I(a) through a first J spacer functional group. The spacer J is attached or joined to the linker X at the second end of spacer J through a second J spacer functional group. Similarly, spacer J′ is attached or joined to the terminal hydrazide nitrogen of the second hydrazide compound moiety as depicted in formulae I and I(a) through a first J′ spacer functional group. The spacer J′ is attached or joined to the linker X′ at the second end of the spacer J′ through a second J′ spacer functional group.

In a specific embodiment of structure I(a), each of R², R^{2′}, R³, R^{3′}, R⁴, R^{4′}, R⁵, R^{5′}, R⁶, R^{6′}, R¹³, R^{13′}, R¹⁴, and R^{14′}, X and X′, and n are as described above and herein for structure I(a), and each of J and J′ is a moiety of structure J1 (4,4′-diisothiocyanostilbene-2,2′-disulfonic acid (DIDS)) and the compound has the following structure I(b):

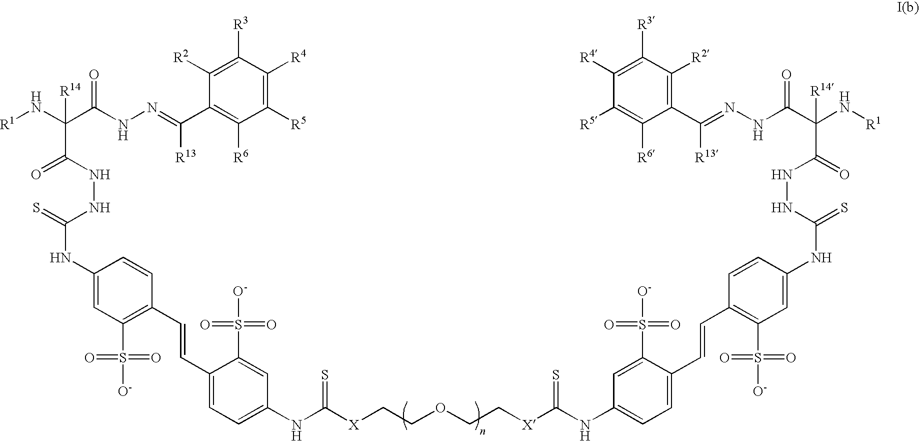

or a pharmaceutically acceptable salt, prodrug, or stereoisomer thereof.

Accordingly, in certain embodiments, R¹ and R^{1′} are the same or different and independently optionally substituted phenyl, optionally substituted heteroaryl, optionally substituted quinolinyl, optionally substituted anthracenyl, or optionally substituted naphthalenyl;

R², R^{2′}, R³, R^{3′}, R⁴, R^{4′}, R⁵, R^{5′}, R⁶, and R^{6′} are each the same or different and independently hydrogen, hydroxy, C₁₋₈ alkyl, C₁₋₈ alkoxy, carboxy, halo, nitro, cyano, —SO₃H, —S(═O)₂NH₂, aryl, and heteroaryl;

R¹³, R^{13′}, R¹⁴, and R^{14′} are each the same or different and independently hydrogen or C₁₋₈ alkyl;

X and X′ are each the same or different linker moiety; and

n is an integer between 0 and 2,500.

In certain embodiments of structures I(b), n is any integer between 0 and 10, between 0 and 100, between 1 and 5, between 1 and 10, between 1 and 100, between 1 and 300, between 1 and 550, between 1 and 1000, between 1 and 2500, between 10 and 2500, between 10 and 2000, between 50 and 1000, between 250 and 1000, or between 450 and 1000. In more specific embodiments of structures I(b), n is any integer between 50 and 1000. In another specific embodiment, n is any integer between 200 and 300. In yet another specific embodiment, n is any integer between 450 and 550. In still another specific embodiment, n is any integer between 900 and 1000. In another specific embodiment, n is 0.

In certain embodiments, R¹³, R^{13′}, R¹⁴, and R^{14′} are the same or different and independently hydrogen or methyl. In a more specific embodiment, each of R¹³, R^{13′}, R¹⁴, and R^{14′} is hydrogen.

In more specific embodiments of structure I(b), R¹ and R^{1′} are the same or different and independently 1-naphthalenyl or 2-naphthalenyl, optionally substituted with one or more of halo, hydroxy, —SH, —SO₃H, C₁₋₈ alkyl, and C₁ s alkoxy; aryloxy; mono-halophenyl; di-halophenyl; mono-alkylphenyl; 2-anthracenyl; or 6-quinolinyl. In a specific embodiment, halo is chloro. In another specific embodiment, C₁₋₈ alkyl is methyl. In other specific embodiments, R¹ and R^{1′} are the same or different and independently quinolinyl or anthracenyl, optionally substituted with one or more of halo, hydroxy, C₁₋₈ alkyl, or C₁₋₈ alkoxy.

In more specific embodiments of structure I(b), R¹ and R^{1′} are the same or different and independently unsubstituted phenyl, or substituted phenyl wherein phenyl is substituted with one or more of hydroxy, C₁₋₈ alkyl, aryl, aryloxy, —SO₃H, C₁₋₈ alkoxy, or halo wherein halo is fluoro, chloro, bromo, or iodo. In a specific embodiment, halo is chloro. In another specific embodiment, C₁₋₈ alkyl is methyl. In yet another specific embodiment, R¹ and R^{1′} are the same or different and independently phenyl substituted with methyl or chloro.

In other specific embodiments of structure I(b), R¹ and R^{1′} are the same or different and independently 2-halophenyl; 4-halophenyl; -2-4-halophenyl, 4-methylphenyl; mono-(halo)naphthalenyl, di-(halo)naphthalenyl, tri-(halo)naphthalenyl, mono-(hydroxy)naphthalenyl, di-(hydroxy)naphthalenyl, tri-(hydroxy)naphthalenyl, mono-(alkoxy)naphthalenyl, di-(alkoxy)naphthalenyl, tri-(alkoxy)naphthalenyl, mono-(aryloxy)naphthalenyl, di-(aryloxy)naphthalenyl, mono-(alkyl)naphthalenyl, di-(alkyl)naphthalenyl, tri-(alkyl)naphthalenyl, mono-(hydroxy)-naphthalene-sulfonic acid, mono-(hydroxy)-naphthalene-disulfonic acid, mono(halo)-mono(hydroxy)naphthalenyl; di(halo)-mono(hydroxy)naphthalenyl; mono(halo)-di(hydroxy)naphthalenyl; di(halo)-di(hydroxy)naphthalenyl; mono-(alkyl)-mono-(alkoxy)-naphthalenyl, mono-(alkyl)-di-(alkoxy)-naphthalenyl, mono-(halo)phenyl, di-(halo)phenyl, tri-(halo) phenyl, mono-(hydroxy)phenyl, di-(hydroxy)phenyl, tri-(hydroxy)phenyl, mono-(alkoxy)phenyl, di-(alkoxy)phenyl, tri-(alkoxy)phenyl, mono-(aryloxy)phenyl, di-(aryloxy)phenyl, mono-(alkyl)phenyl, di-(alkyl)phenyl, tri-(alkyl)phenyl, mono-(hydroxy)-phenyl-sulfonic acid, mono-(hydroxy)-phenyl-disulfonic acid, mono(halo)-mono(hydroxy)phenyl, di(halo)-mono(hydroxy)phenyl, mono(halo)-di(hydroxy)phenyl, di(halo)-di(hydroxy)phenyl, mono-(alkyl)-mono-(alkoxy)-phenyl, or mono-(alkyl)-di-(alkoxy)-phenyl wherein halo is fluoro, chloro, bromo, or iodo. In a particular embodiment, halo is chloro.

In even more specific embodiments of structure I(b), R¹ and R^{1′} are the same or different and independently 2-naphthalenyl, 2-chlorophenyl, 4-chlorophenyl, 2-4-dichlorophenyl, 4-methylphenyl, 2-anthracenyl, or 6-quinolynyl. In other specific embodiments, of structure I(b), R¹ and R^{1′} are each the same or different and independently 2-naphthalenyl or 4-chlorophenyl.

In other specific embodiments of structure I(b) as described above, R², R^{2′}, R³, R^{3′}, R⁴, R^{4′}, R⁵, R^{5′}, R⁶, and R^{6′} are each the same or different and independently hydrogen, hydroxy, halo, C₁₋₈ alkyl, C₁₋₈ alkoxy, or carboxy.

In other certain embodiments of structure I(b), R², R³, R⁴, R⁵, and R⁶, are each the same or different and independently selected from hydrogen, hydroxy, halo, C₁₋₈ alkyl, C₁₋₈ alkoxy, or carboxy, such that the phenyl group to which R², R³, R⁴, R⁵, and R⁶ are attached is substituted with one, two, or three halo; one or two carboxy; one, two, or three hydroxy; one or two halo and one, two, or three hydroxy; one or two halo, one or two hydroxy, and one C₁₋₈ alkoxy; one or two halo, one hydroxy, and one or two C₁₋₈ alkoxy; or one halo, one or two hydroxy, and one or two C₁₋₈ alkoxy, wherein halo is bromo, chloro, iodo, or fluoro; in a more specific embodiment, halo is bromo.

In other certain embodiments of structure I(b), R^{2′}, R^{3′}, R^{4′}, R^{5′}, and R^{6′} are each the same or different and independently selected from hydrogen, hydroxy, halo, C₁₋₈ alkyl, C₁₋₈ alkoxy, or carboxy, such that the phenyl group to which R^{2′}, R^{3′}, R^{4′}, R^{5′}, and R^{6′} are attached is substituted with one, two, or three halo; one or two carboxy; one, two, or three hydroxy; one or two halo and one, two, or three hydroxy; one or two halo, one or two hydroxy, and one C₁₋₈ alkoxy; one or two halo, one hydroxy, and one or two C₁₋₈ alkoxy; or one halo, one or two hydroxy, and one or two C₁₋₈ alkoxy, wherein halo is bromo, chloro, iodo, or fluoro. In a more specific embodiment, halo is bromo.

In certain specific embodiments of structure I(b), R², R³, R⁴, R⁵, and R⁶ are the same or different and independently selected from hydrogen, hydroxy, halo, C₁₋₈ alkyl, C₁₋₈ alkoxy, or carboxy, such that the phenyl group to which R², R³, R⁴, R⁵, and R⁶ are attached is substituted with di(hydroxy); mono-(halo)-mono-(hydroxy); mono-(halo)-di-(hydroxy); mono-(halo)-tri-(hydroxy); di(halo)-mono-(hydroxy); di(halo)-di-(hydroxy); di(halo)-tri-(hydroxy); mono-(halo)-mono-(hydroxy)-mono-(alkoxy); mono-(halo)-di-(hydroxy)-mono-(alkoxy); mono-(halo)-mono-(hydroxy)-di-(alkoxy); mono-(halo)-di-(hydroxy)-di-(alkoxy); di-(halo)-mono-(hydroxy)-mono-(alkoxy); di-(halo)-di-(hydroxy)-mono-(alkoxy); or di-(halo)-mono-(hydroxy)-di-(alkoxy). In a specific embodiment, halo is bromo. In other specific embodiments, alkoxy is methoxy.

In certain specific embodiments of structure I(b), R^{2′}, R^{3′}, R^{4′}, R^{5′}, and R^{6′}, are the same or different and independently selected from hydrogen, hydroxy, halo, C₁₋₈ alkyl, C₁₋₈ alkoxy, or carboxy, such that the phenyl group to which R^{2′}, R^{3′}, R^{4′}, R^{5′}, and R^{6′} is attached is substituted with di(hydroxy); mono-(halo)-mono-(hydroxy); mono-(halo)-di-(hydroxy); mono-(halo)-tri-(hydroxy); di(halo)-mono-(hydroxy); di(halo)-di-(hydroxy); di(halo)-tri-(hydroxy); mono-(halo)-mono-(hydroxy)-mono-(alkoxy); mono-(halo)-di-(hydroxy)-mono-(alkoxy); mono-(halo)-mono-(hydroxy)-di-(alkoxy); mono-(halo)-di-(hydroxy)-di-(alkoxy); di-(halo)-mono-(hydroxy)-mono-(alkoxy); di-(halo)-di-(hydroxy)-mono-(alkoxy); or di-(halo)-mono-(hydroxy)-di-(alkoxy). In a specific embodiment, halo is bromo. In other specific embodiments, alkoxy is methoxy.

In other certain specific embodiments of structure I(b), R², R³, R⁴, R⁵, and R⁶ are the same or different and independently selected from hydrogen, hydroxy, halo, C₁₋₈ alkyl, C₁₋₈ alkoxy, or carboxy, such that the phenyl group to which R², R³, R⁴, R⁵, and R⁶ is attached is 2-, 3-, or 4-halophenyl; 3,5-dihalophenyl; 2-, 3-, or 4-hydroxyphenyl; 2,4-dihydroxyphenyl; 3,5-dihalo-2,4,6-trihydroxyphenyl, 3,5-dihalo-2,4-dihydroxyphenyl; 3,5-dihalo-4-hydroxyphenyl; 3-halo-4-hydroxyphenyl; 3,5-dihalo-2-hydroxy-4-methoxyphenyl; or 4-carboxyphenyl, wherein halo is bromo, chloro, fluoro, or iodo. In another specific embodiment, halo is bromo.

In other certain specific embodiments of structure I(b), R^{2′}, R^{3′}, R^{4′}, R^{5′}, and R^{6′} are the same or different and independently selected from hydrogen, hydroxy, halo, C₁₋₈ alkyl, C₁₋₈ alkoxy, or carboxy, such that the phenyl group to which R^{2′}, R^{3′}, R^{4′}, R^{5′}, and R^{6′} is attached is 2-, 3-, or 4-halophenyl; 3,5-dihalophenyl; 2-, 3-, or 4-hydroxyphenyl; 2,4-dihydroxyphenyl; 3,5-dihalo-2,4,6-trihydroxyphenyl, 3,5-dihalo-2,4-dihydroxyphenyl; 3,5-dihalo-4-hydroxyphenyl; 3-halo-4-hydroxyphenyl; 3,5-dihalo-2-hydroxy-4-methoxyphenyl; or 4-carboxyphenyl, wherein halo is bromo, chloro, fluoro, or iodo. In another specific embodiment, halo is bromo.

In other certain specific embodiments of structure I(b), each of R³ and R⁵ is halo and each of R⁴ and R⁶ is hydroxy. In another specific embodiment, each of R³ and R⁵ is halo and R⁴ is hydroxy. In yet another specific embodiment, R³ and R⁵ is bromo and each of R⁴ and R⁶ is hydroxy. In still another specific embodiment, R³ and R⁵ is bromo, R⁴ is hydroxy, and R⁶ is hydrogen. In certain specific embodiments of structure I(b), each of R^{3′} and R^{5′} is halo and each of R^{4′} and R^{6′} is hydroxy. In another specific embodiment, each of R^{3′} and R^{5′} is halo and R^{4′} is hydroxy. In still another specific embodiment, each of R^{3′} and R^{5′} is bromo and each of R^{4′} and R^{6′} is hydroxy. In yet another specific embodiment, each of R^{3′} and R^{5′} is bromo, R^{4′} is hydroxy, and R^{6′} is hydrogen. In specific embodiments, each of R² and R^{2′} is hydrogen.

In certain specific embodiments of structure I(b), each of R³, R^{3′}, R⁵ and R^{5′} is halo and each of R⁴, R^{4′}, R⁶, and R^{6′} is hydroxy. In specific embodiments, each of R² and R^{2′} is hydrogen.

In other specific embodiments of structure I(b), each of R³, R^{3′}, R⁵, and R^{5′} is halo and each of R⁴ and R^{4′} is hydroxy. In specific embodiments, each of R² and R^{2′} is hydrogen.

In yet more specific embodiments of structure I(b), each of R³, R^{3′}, R⁵, and R^{5′} is bromo, and each of R⁴, R^{4′}, R⁶, and R^{6′} is hydroxy. In specific embodiments, each of R² and R^{2′} is hydrogen.

In yet more specific embodiments of structure I(b), each of R³, R^{3′}, R⁵, and R^{5′} is bromo, each of R⁴ and R^{4′} is hydroxy, and each of R⁶ and R^{6′} is hydrogen. In specific embodiments, each of R² and R^{2′} is hydrogen.

In other more specific embodiments of structure I(b) as described above, R¹³, R^{13′}, R¹⁴, and R^{14′} are the same or different and independently hydrogen or methyl. In such embodiments, R¹ and R^{1′} are each the same or different and independently phenyl substituted with at least one chloro or methyl; 1-naphthalenyl; 2-naphthalenyl; 6-quinolinyl; or 2-anthracenyl. In specific embodiments, R², R³, R⁴, R⁵, R⁶, R^{2′}, R^{3′}, R^{4′}, R^{5′}, and R^{6′} are each the same or different and independently hydrogen, halo, methoxy, hydroxyl, or carboxy; in specific embodiments, halo is bromo. In certain specific embodiments, when each of R³, R⁴, R⁵, R⁶, R^{3′}, R^{4′}, R^{5′}, and R^{6′} is not hydrogen, R² and R^{2′} are each hydrogen.

With respect to the embodiments of structure I(b), the linker moieties X and X′ are each a functional group that may be used for conjugating the spacer J and spacer J′, respectively, to polyethylene glycol (i.e., (—CH₂—O—CH₂—)ₙ). In certain specific embodiments of the compounds having structure I(b) as described above, the linker X and the linker X′ are the same or different and independently —NH—, —O—, or —S—. In a particular embodiment, X and X′ are the same and each is —NH—.

In certain specific embodiments of structures I, I(a) and I(b), the compounds are sodium salts.

In yet more specific embodiments of structure I, I(a) and I(b) that are described above, the compounds are illustrated by the following structures I(c)-I(j):

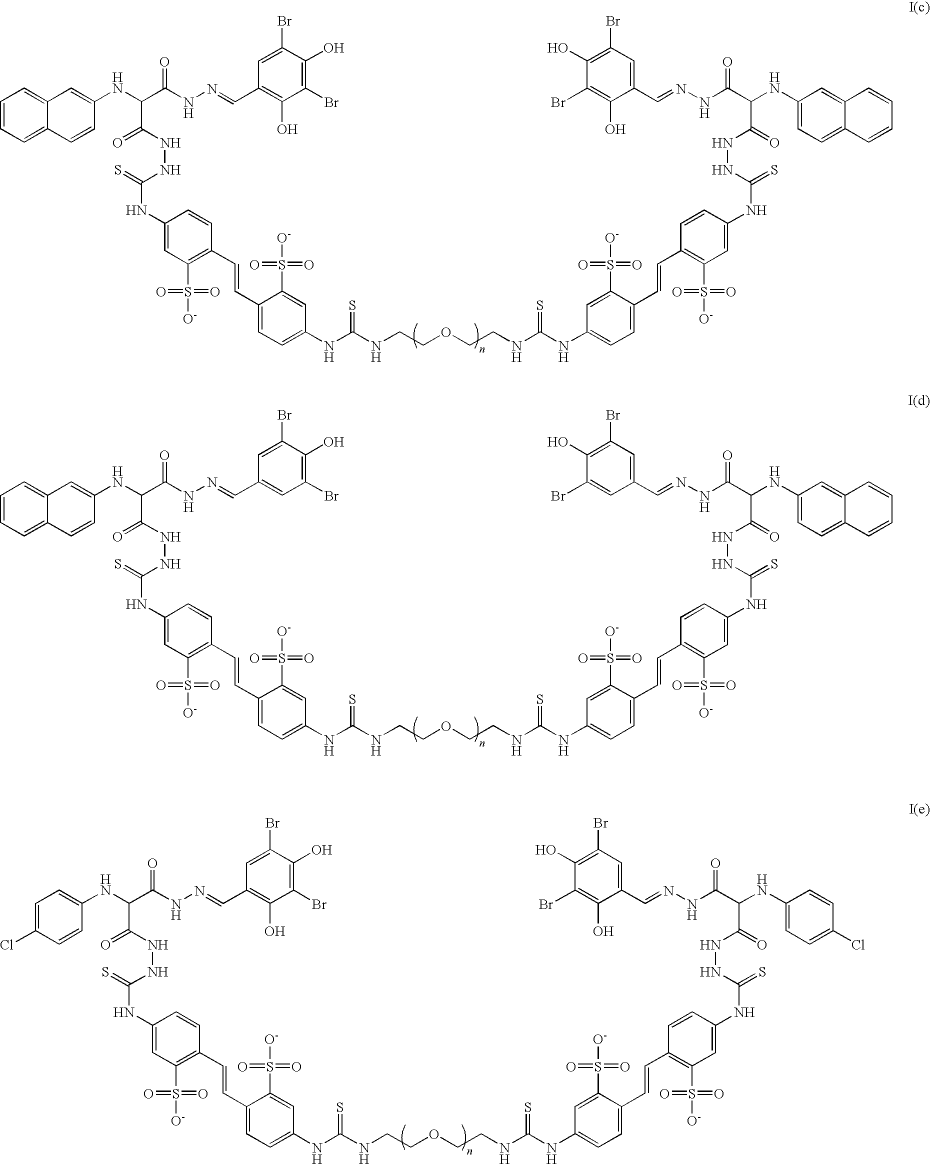

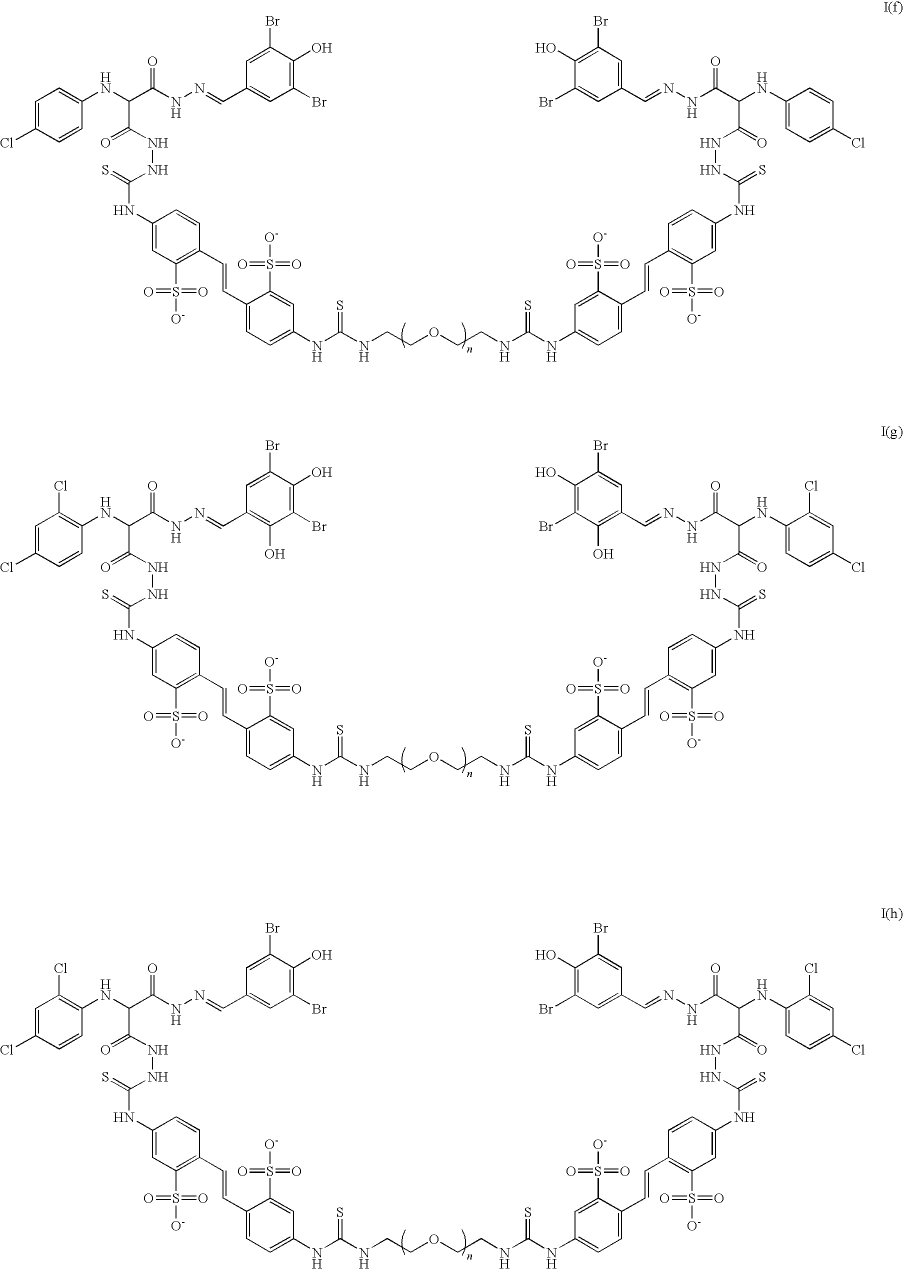

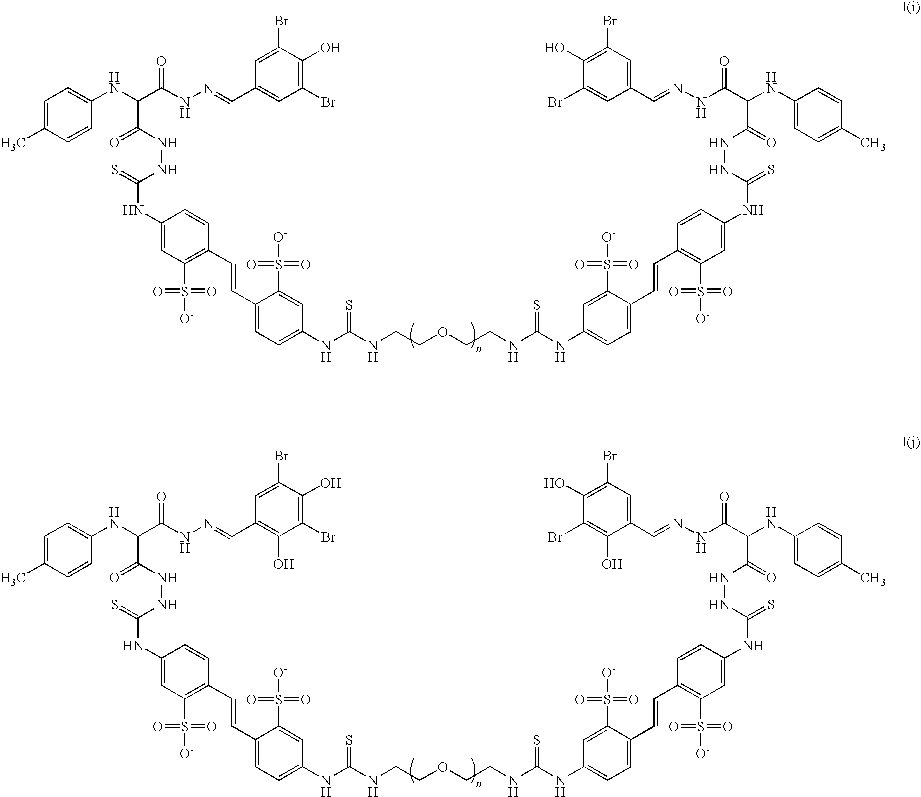

In certain specific embodiments, structures I(c)-I(j) are sodium salts.

In certain embodiments of a structure of any of formulae I(b), I(c), I(d), I(e), I(f), I(g), I(h), I(i), and I(j), n is any integer between 0 and 10, between 0 and 100, between 1 and 5, between 1 and 10, between 1 and 100, between 1 and 300, between 1 and 550, between 1 and 1000, between 1 and 2500, between 10 and 2500, between 10 and 2000, between 50 and 1000, between 250 and 1000, or between 450 and 1000. In more specific embodiments of structures I(b), and structures I(c)-I(j), n is any integer between 50 and 1000. In another specific embodiment, n is any integer between 200 and 300. In yet another specific embodiment, n is any integer between 450 and 550. In still another specific embodiment, n is any integer between 900 and 1000. In another specific embodiment, n is 0.

The conjugate compounds having a structure of any one of formulae I, I(a), I(b), and structures I(c)-I(j) or any substructure thereof are also referred to herein as divalent malonic hydrazide-PEG conjugate compounds (or divalent malonic hydrazide-PEG conjugates).

### Divalent Glycine Hydrazide Polymer Conjugates

Also provided herein are compounds that are divalent glycine hydrazide polymer conjugates. Such compounds are also useful as inhibitors of the cystic fibrosis transmembrane conductance regulator (CFTR) chloride channel and have the following structure II:

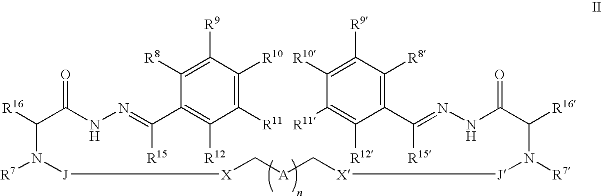

or a pharmaceutically acceptable salt, prodrug, or stereoisomer thereof

wherein:

R⁷ and R^{7′} are the same or different and independently optionally substituted phenyl, optionally substituted heteroaryl, optionally substituted quinolinyl, optionally substituted anthracenyl, or optionally substituted naphthalenyl;
R⁸, R^{8′}, R⁹, R^{9′}, R¹⁰, R^{10′}, R¹¹, R^{11′}, R¹², and R^{12′} are the same or different and independently hydrogen, hydroxy, C₁₋₈ alkyl, C₁₋₈ alkoxy, carboxy, halo, nitro, cyano, —SO₃H, —S(═O)₂NH₂, aryl, and heteroaryl;

R¹⁵, R^{15′}, R¹⁶ and R^{16′} are the same or different and independently hydrogen, oxo, or C₁₋₈ alkyl;

X and X′ are each the same or different linker moiety;

J and J′ are each the same or different spacer moiety;

A is a polymer subunit; and

n is an integer between 0 and 2,500.

In certain embodiments, n is any integer between 0 and 10, between 0 and 100, between 1 and 5, between 1 and 10, between 1 and 100, between 1 and 300, between 1 and 550, between 1 and 1000, between 1 and 2500, between 10 and 2500, between 10 and 2000, between 50 and 1000, between 250 and 1000, or between 450 and 1000. In more specific embodiments of structures I, n is any integer between 50 and 1000. In another specific embodiment, n is any integer between 200 and 300. In yet another specific embodiment, n is any integer between 450 and 550. In still another specific embodiment, n is any integer between 900 and 1000. In another specific embodiment, n is 0.

In certain embodiments, A is a subunit of the polymer polyethylene glycol (PEG) (i.e., —CH₂—O—CH₂—). In another embodiment, A is a subunit of a polymer selected from a polyethylenamine (PEI), a carbohydrate, such as a dextran, wherein the polymer has two termini (i.e., terminal ends) one of which is joined to linker X and the other (or second) of which is joined to the linker X′. In other embodiments, A is an amino acid and the polymer is a peptide or polypeptide. In certain specific embodiments, when A is an amino acid, n is between 1 and 5, 1 and 10, 1 and 15, 1 and 20, 1 and 40, 1 and 50, between 1 and 100, or between 100 and 500.

In another specific embodiment, A is —CH₂—NH—CH₂—. In certain specific embodiments, n is an integer between 1 and 5, 1 and 10, 1 and 20, 1 and 30, between 1 and 100, between 100 and 500, or between 500 and 1000.

In another embodiment, A is optionally substituted alkanediyl, optionally substituted alkenylene (divalent aliphatic hydrocarbon containing at least one double bond), or optionally substituted alkynylene (divalent aliphatic hydrocarbon containing at least one triple bond). In certain specific embodiments, when A is an alkanediyl, alkenylene, or alkynylene n is an integer between 2 and 5, 2 and 10, 2 and 20, or between 2 and 30, or between 2 and 50.

In still other embodiments, A is optionally substituted aryl or optionally substituted cycloalkyl. In specific embodiments, A is optionally substituted phenyl, and in other specific embodiments, A is optionally substitute cyclohexyl. In certain specific embodiments, when A is an aryl or cycloalkyl, n is an integer between 1 and 3, 1 and 5, 1 and 10, 1 and 20, or 1 and 30, or between 1 and 100.

In certain embodiments, each of R⁷, R^{7′}, R⁸, R^{8′}, R⁹, R^{9′}, R¹⁰, R^{10′}, R¹¹, R^{11′}, R¹², and R^{12′}, R¹⁵, R^{15′}, R¹⁶, R^{16′}, X, X′, J, and J′ are as defined herein (see below with respect to divalent glycine hydrazide-PEG conjugate compounds).

### Divalent Glycine Hydrazide PEG Conjugates

In yet another embodiment, n is 0 and A is absent. In one embodiment, A is —CH₂—O—CH₂— and the polymer is polyethylene glycol (PEG). Provided herein are compounds that are divalent glycine hydrazide PEG conjugates. Such compounds are also useful as inhibitors of the cystic fibrosis transmembrane conductance regulator (CFTR) chloride channel and have the following structure II(a):

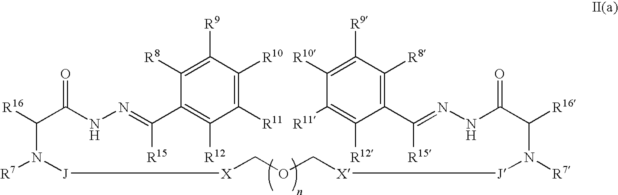

or a pharmaceutically acceptable salt, prodrug, or stereoisomer thereof wherein:

R⁷ and R^{7′} are each the same or different and independently optionally substituted phenyl, optionally substituted heteroaryl, optionally substituted quinolinyl, optionally substituted anthracenyl, or optionally substituted naphthalenyl;

R⁸, R^{8′}, R⁹, R^{9′}, R¹⁰, R^{10′}, R¹¹, R^{11′}, R¹², and R^{12′} are each the same or different and independently hydrogen, hydroxy, C₁₋₈ alkyl, C₁₋₈ alkoxy, carboxy, halo, nitro, cyano, —SO₃H, —S(═O)₂NH₂, aryl, and heteroaryl;

R¹⁵, R^{15′}, R¹⁶, and R^{16′} are each the same or different and independently hydrogen, oxo, or C₁₋₈ alkyl;

X and X′ are each the same or different linker moiety;

J and J′ are each the same or different spacer moiety; and

n is an integer between 0 and 2,500.

In certain embodiments of compounds of structure II(a), n is any integer between 0 and 10, between 0 and 100, between 1 and 5, between 1 and 10, between 1 and 100, between 1 and 300, between 1 and 550, between 1 and 1000, between 1 and 2500, between 10 and 2500, between 10 and 2000, between 50 and 1000, between 250 and 1000, or between 450 and 1000. In more specific embodiments of structure II and structure II(a), n is any integer between 50 and 1000. In another specific embodiment, n is any integer between 200 and 300. In yet another specific embodiment, n is any integer between 450 and 550. In still another specific embodiment, n is any integer between 900 and 1000. In another specific embodiment, n is 0.

In a particular embodiment of structure II and structure II(a), R⁷ and R^{7′} are the same or different and independently unsubstituted phenyl, or substituted phenyl wherein phenyl is substituted with one or more of hydroxy, C₁₋₈ alkyl, aryl, aryloxy, —SO₃H, C₁₋₈ alkoxy, or halo wherein halo is fluoro, chloro, bromo, or iodo. In a specific embodiment, halo is chloro.

In another particular embodiment of structure II and structure II(a), R⁷ and R^{7′} are the same or different and independently 1-naphthalenyl or 2-naphthalenyl, optionally substituted with one or more of halo, hydroxy, —SH, —SO₃H, C₁₋₈ alkyl, and C₁₋₈ alkoxy; aryloxy; mono-halophenyl; di-halophenyl; mono-alkylphenyl; 2-anthracenyl; or 6-quinolinyl. In a specific embodiment, C₁₋₈ alkyl is methyl. In other specific embodiments, halo is chloro.

In another particular embodiment of structure II and structure II(a), R⁷ and R^{7′} are the same or different and independently are the same or different and independently 2-halophenyl; 4-halophenyl; -2-4-halophenyl, 4-methylphenyl; mono-(halo)naphthalenyl, di-(halo)naphthalenyl, tri-(halo)naphthalenyl, mono-(hydroxy)naphthalenyl, di-(hydroxy)naphthalenyl, tri-(hydroxy)naphthalenyl, mono-(alkoxy)naphthalenyl, di-(alkoxy)naphthalenyl, tri-(alkoxy)naphthalenyl, mono-(aryloxy)naphthalenyl, di-(aryloxy)naphthalenyl, mono-(alkyl)naphthalenyl, di-(alkyl)naphthalenyl, tri-(alkyl)naphthalenyl, mono-(hydroxy)-naphthalene-sulfonic acid, mono-(hydroxy)-naphthalene-disulfonic acid, mono(halo)-mono(hydroxy)naphthalenyl; di(halo)-mono (hydroxy)naphthalenyl; mono(halo)-di(hydroxy)naphthalenyl; di(halo)-di(hydroxy)naphthalenyl; mono-(alkyl)-mono-(alkoxy)-naphthalenyl, mono-(alkyl)-di-(alkoxy)-naphthalenyl, mono-(halo)phenyl, di-(halo)phenyl, tri-(halo) phenyl, mono-(hydroxy)phenyl, di-(hydroxy)phenyl, tri-(hydroxy)phenyl, mono-(alkoxy)phenyl, di-(alkoxy)phenyl, tri-(alkoxy)phenyl, mono-(aryloxy)phenyl, di-(aryloxy)phenyl, mono-(alkyl)phenyl, di-(alkyl)phenyl, tri-(alkyl)phenyl, mono-(hydroxy)-phenyl-sulfonic acid, mono-(hydroxy)-phenyl-disulfonic acid, mono(halo)-mono(hydroxy)phenyl, di(halo)-mono(hydroxy)phenyl, mono(halo)-di(hydroxy)phenyl, di(halo)-di(hydroxy)phenyl, mono-(alkyl)-mono-(alkoxy)-phenyl, or mono-(alkyl)-di-(alkoxy)-phenyl wherein halo is fluoro, chloro, bromo, or iodo. In a particular embodiment, halo is chloro.

In yet another embodiment of structure II and structure II(a), R⁷ and R^{7′} are the same or different and independently quinolinyl or anthracenyl, optionally substituted with one or more of halo, hydroxy, C₁₋₈ alkyl, or C₁₋₈ alkoxy. In a specific embodiment, C₁₋₈ alkyl is methyl. In other specific embodiments, halo is chloro. In another specific embodiment of structure II and structure II(a), R⁷ and R^{7′} are the same or different and independently substituted phenyl wherein phenyl is substituted with methyl or chloro.

In still another embodiment of structure II and structure II(a), R⁷ and R^{7′} are the same or different and independently 2-naphthalenyl or 1-naphthalenyl, optionally substituted with one or more of halo, hydroxy, —SH, —SO₃H, C₁₋₈ alkyl, aryl, aryloxy, or C₁₋₈ alkoxy.

In certain embodiments of structure II and structure II(a), R⁷ and R^{7′} are the same or different and independently mono-(halo)naphthalenyl; di-(halo)naphthalenyl; tri-(halo)naphthalenyl; mono-(hydroxy)naphthalenyl; di-(hydroxy)naphthalenyl; tri-(hydroxy)naphthalenyl; mono-(alkoxy)naphthalenyl; di-(alkoxy)naphthalenyl; tri-(alkoxy)naphthalenyl; mono-(aryloxy)naphthalenyl; di-(aryloxy)naphthalenyl; mono-(alkyl)naphthalenyl; di-(alkyl)naphthalenyl; tri-(alkyl)naphthalenyl; mono-(hydroxy)-naphthalene-sulfonic acid; mono-(hydroxy)-naphthalene-disulfonic acid; mono(halo)-mono(hydroxy)naphthalenyl; di(halo)-mono(hydroxy)naphthalenyl; mono(halo)-di(hydroxy)naphthalenyl; di(halo)-di(hydroxy)naphthalenyl; mono-(alkyl)-mono-(alkoxy)-naphthalenyl; or mono-(alkyl)-di-(alkoxy)-naphthalenyl, wherein halo is fluoro, chloro, bromo, or iodo. In a specific embodiment, halo is chloro.

In yet other specific embodiments of structure II and structure II(a), R⁷ and R^{7′} are the same or different and independently 2-naphthalenyl, 2-chlorophenyl, 4-chlorophenyl, 2,4-chlorophenyl, 4-methylphenyl, 2-anthracenyl, or 6-quinolinyl.

In other particular embodiments of structure II and structure II(a), R⁷ and R^{7′} are the same or different and independently quinolinyl or anthracenyl, optionally substituted with one or more of halo, hydroxy, C₁₋₈ alkyl, or C₁₋₈ alkoxy.

In other particular embodiments of structure II and structure II(a) described above, R⁸, R^{8′}, R⁹, R^{9′}, R¹⁰, R^{10′}, R¹¹, R^{11′}, R¹², and R^{12′} are the same or different and independently hydrogen, hydroxy, halo, carboxy, C₁₋₈ alkyl, or C₁₋₈ alkoxy.

In another particular embodiment of structure II and structure II(a), R⁸, R⁹, R¹⁰, R^{10′}, R¹¹, and R¹² are each the same or different and independently selected from hydrogen, hydroxy, halo, carboxy, C₁₋₈ alkyl, or C₁₋₈ alkoxy, such that the phenyl group to which R⁸, R⁹, R¹⁰, R¹¹, and R¹² are attached is substituted with one, two, or three halo; one or two carboxy; one, two, or three hydroxy; one or two halo and one, two, or three hydroxy; one or two halo, one or two hydroxy, and one C₁₋₈ alkoxy; one or two halo, one hydroxy, and one or two C₁₋₈ alkoxy; or one halo, one or two hydroxy, and one or two C₁₋₈ alkoxy.

In another particular embodiment of structure II and structure II(a), R^{8′}, R^{9′}, R^{10′}, R^{11′}, and R^{12′} are each the same or different and independently selected from hydrogen, hydroxy, halo, carboxy, C₁₋₈ alkyl, or C₁₋₈ alkoxy, such that the phenyl group to which R^{8′}, R^{9′}, R^{10′}, R^{11′}, and R^{12′} are attached is substituted with one, two, or three halo; one or two carboxy; one, two, or three hydroxy; one or two halo and one, two, or three hydroxy; one or two halo, one or two hydroxy, and one C₁₋₈ alkoxy; one or two halo, one hydroxy, and one or two C₁₋₈ alkoxy; or one halo, one or two hydroxy, and one or two C₁₋₈ alkoxy.

In yet other specific embodiments of structure II and structure II(a), R⁸, R⁹, R¹⁰, R¹, and R¹² are each the same or different and independently selected from hydrogen, hydroxy, halo, carboxy, C₁₋₈ alkyl, or C₁₋₈ alkoxy, such that the phenyl group to which R⁸, R⁹, R¹⁰, R¹¹, and R¹² is attached is substituted with di(hydroxy); mono-(halo)-mono-(hydroxy); mono-(halo)-di-(hydroxy) mono-(halo)-tri-(hydroxy); di(halo)-mono-(hydroxy); di(halo)-di-(hydroxy); di(halo)-tri-(hydroxy); mono-(halo)-mono-(hydroxy)-mono-(alkoxy);mono-(halo)-di-(hydroxy)-mono-(alkoxy); mono-(halo)-mono-(hydroxy)-di-(alkoxy); mono-(halo)-di-(hydroxy)-di-(alkoxy); di-(halo)-mono-(hydroxy)-mono-(alkoxy); di-(halo)-di-(hydroxy)-mono-(alkoxy); or di-(halo)-mono-(hydroxy)-di-(alkoxy). In specific embodiments, halo is bromo. In other specific embodiments, alkoxy is methoxy.

In yet other specific embodiments of structure II and structure II(a), R^{8′}, R^{9′}, R^{10′}, R^{11′}, and R^{12′} are each the same or different and independently selected from hydrogen, hydroxy, halo, carboxy, C₁₋₈ alkyl, or C₁₋₈ alkoxy, such that the phenyl group to which R^{8′}, R^{9′}, R^{10′}, R^{11′}, and R^{12′} are attached is substituted with di(hydroxy); mono-(halo)-mono-(hydroxy); mono-(halo)-di-(hydroxy) mono-(halo)-tri-(hydroxy); di(halo)-mono-(hydroxy); di(halo)-di-(hydroxy); di(halo)-tri-(hydroxy); mono-(halo)-mono-(hydroxy)-mono-(alkoxy);mono-(halo)-di-(hydroxy)-mono-(alkoxy); mono-(halo)-mono-(hydroxy)-di-(alkoxy); mono-(halo)-di-(hydroxy)-di-(alkoxy); di-(halo)-mono-(hydroxy)-mono-(alkoxy); di-(halo)-di-(hydroxy)-mono-(alkoxy); or di-(halo)-mono-(hydroxy)-di-(alkoxy). In specific embodiments, halo is bromo. In other specific embodiments, alkoxy is methoxy.

In certain specific embodiments of structure TI and structure II(a), R⁸, R⁹, R¹⁰, R¹¹, and R¹² are each the same or different and independently selected from hydrogen, hydroxy, halo, carboxy, C₁₋₈ alkyl, or C₁₋₈ alkoxy, such that the phenyl group to which R⁸, R⁹, R¹⁰, R¹¹, and R¹² are attached is 2-, 3-, or 4-halophenyl; 3,5-dihalophenyl; 2-, 3-, or 4-hydroxyphenyl; 2,4-dihydroxyphenyl; 3,5-dihalo-2,4,6-trihydroxyphenyl; 3,5-dihalo-2,4-dihydroxyphenyl; 3,5-dihalo-4-hydroxyphenyl; 3-halo-4-hydroxyphenyl; 3,5-dihalo-2-hydroxy-4-methoxyphenyl; or 4-carboxyphenyl. In a more specific embodiment, the halo is bromo.

In other certain specific embodiments of structure II and structure II(a), R^{8′}, R^{9′}, R^{10′}, R^{11′}, and R^{12′} are each the same or different and independently selected from hydrogen, hydroxy, halo, carboxy, C₁₋₈ alkyl, or C₁₋₈ alkoxy, such that the phenyl group to which R^{8′}, R^{9′}, R^{10′}, R^{11′}, and R^{12′} are attached is 2-, 3-, or 4-halophenyl; 3,5-dihalophenyl; 2-, 3-, or 4-hydroxyphenyl; 2,4-dihydroxyphenyl; 3,5-dihalo-2,4,6-trihydroxyphenyl, 3,5-dihalo-2,4-dihydroxyphenyl; 3,5-dihalo-4-hydroxyphenyl; 3-halo-4-hydroxyphenyl; 3,5-dihalo-2-hydroxy-4-methoxyphenyl; or 4-carboxyphenyl, wherein halo is fluoro, chloro, bromo, or iodo. In a more specific embodiment, the halo is bromo.

In a more specific embodiment of structure II and structure II(a), each of R⁹ and R¹¹ is halo and each of R¹⁰ and R¹² is hydroxy. In another specific embodiment, each of R⁹ and R¹¹ is halo and R¹⁰ is hydroxy. In still another specific embodiment, each of R⁹ and R¹¹ is bromo, and each of R¹⁰ and R¹² is hydroxy. In yet another specific embodiment, each of R⁹ and R^{11′} is bromo, R¹⁰ is hydroxy, and R¹² is hydrogen. In other embodiments, each of R^{9′} and R^{11′} is halo and each of R^{10′} and R^{12′} is hydroxy. In still other specific embodiments, each of R^{9′} and R^{11′} is halo and R^{10′} is hydroxy. In another particular embodiment, each of R^{9′} and R^{11′} is bromo, and each of R^{10′} and R^{12′} is hydroxy. In still another particular embodiment, each of R^{9′} and R^{11′} is bromo, R^{10′} is hydroxy, and R^{12′} is hydrogen. In other specific embodiments, R⁸ and R^{8′} are each hydrogen.

In certain specific embodiments of structure II and structure II(a), each of R⁹, R^{9′}, R¹¹ and R^{11′} is halo and each of R¹⁰, R^{10′}, R¹², and R^{12′} is hydroxy. In other specific embodiments, R⁸ and R^{8′} are each hydrogen.

In other specific embodiments of structure II and structure II(a), each of R⁹, R^{9′}, R¹¹, and R^{11′} is halo and each of R¹⁰ and R^{10′} is hydroxy. In other specific embodiments, R⁸ and R^{8′} are each hydrogen.

In yet more specific embodiments of structure II and structure II(a), each of R⁹, R^{9′}, R¹¹, and R^{11′} is bromo, and each of R¹⁰, R^{10′}, R¹², and R^{12′} is hydroxy. In other specific embodiments, R⁸ and R^{8′} are each hydrogen.

In yet more specific embodiments of structure II and structure II(a), each of R⁹, R^{9′}, R¹¹, and R^{11′} is bromo, each of R¹⁰ and R^{10′}is hydroxy, and each of R¹² and R^{12′} is hydrogen. In other specific embodiments, R⁸ and R^{8′} are each hydrogen.

In yet other specific embodiments of structure II and structure II(a), R¹⁵, R^{15′}, R¹⁶, and R^{16′} are each the same or different and independently hydrogen or methyl. In another specific embodiment, R¹⁵, R^{15′}, R¹⁶, and R^{16′} are each hydrogen. In still another specific embodiment, each of R¹⁶ and R^{16′} is the same or different and independently hydrogen or oxo.

In other more specific embodiments of structure II and II(a) described above and herein, R¹⁵, R^{15′}, R¹⁶, and R^{16′} are the same or different and independently hydrogen or methyl. In still another specific embodiment, each of R¹⁶ and R^{16′} is oxo. In such embodiments, R⁷ and R^{7′} are each the same or different and independently phenyl substituted with at least one chloro or methyl; 1-naphthalenyl; 2-naphthalenyl; 6-quinolinyl; or 2-anthracenyl. In specific embodiments, R⁸, R⁹, R¹⁰, R¹¹, R¹², R^{8′}, R^{9′}, R^{10′}, R^{11′}, and R^{12′} are each the same or different and independently hydrogen, halo, methoxy, hydroxyl, or carboxy; in specific embodiments, halo is bromo. In certain specific embodiments, when each of R⁹, R¹⁰, R¹¹, R¹², R^{9′}, R^{10′}, R^{11′} and R^{12′} is not hydrogen, R⁸ and R^{8′} are each hydrogen.

The linker moieties X and X′ are each a functional group that may be used for conjugating the spacer J and spacer J′, respectively, to polyethylene glycol (i.e., (—CH₂—O—CH₂—)ₙ) for the compounds having structure II(a) or to the polymer (A), for compounds having structure II. In certain embodiments of the compounds having structure II or II(a) as described above, the linker X and the linker X′ are the same or different and independently —NH—, —O—, —S—. In a more specific embodiment, the linker X and the linker X′ are each —NH—.

The spacer J and the spacer J′ are each independently a moiety that is a spacer between the polyethylene glycol moiety and each of two hydrazide compound moieties (which spacers are respectively conjugated to PEG via the linker X and X′), respectively, as set forth in the structure of formula II(a). Similarly, the spacer J and the spacer J′ are each independently a moiety that is a spacer between the polymer (A), and each of two hydrazide compound moieties (which spacers are respectively conjugated to the polymer via the linker X and X′), respectively, as set forth in the structure of formula II. Exemplary spacer moieties include the structures J1 through J29 as depicted in the table above. Each spacer J and spacer J′ is the same or different and may be selected from J1-J29 (see above). In specific embodiments of a compound of structure II or II(a), each of J and J′ is (4,4′-diisothiocyanostilbene-2,2′-disulfonic acid (DIDS)).

The exemplary structures shown above provide the chemical moiety that may be used as spacer J or spacer J′. As will be readily apparent to a person skilled in the chemical art, the structure of the spacer, such as any one of J1-J29, shown above and herein, will not be identical when the spacer is joined to the hydrazide moiety and to the linker moiety; that is, the above structures J 1-J29 represent a precursor structure of the spacer moieties or in certain instances, represent the reactant chemical moiety. The exemplary spacer moieties J1-J29 above, and other spacer moieties available in the art, have at least two reactive groups (i.e., functional groups), one of which is joined to one of the two hydrazide compounds of the dimer conjugate, and the other (or second) reactive group of the spacer is joined to the linker X (or to the linker X′). As used herein, an “end” of the spacer J and spacer J′ denotes each reactive group (i.e., functional group).

Each spacer J and spacer J′ and has a first end and a second end. The first end of the spacer J is attached or joined to the R⁷ nitrogen through a first J spacer functional group, and the first end of the spacer J′ is attached to the R^{7′} nitrogen through a first J′ spacer functional group. The spacer J is attached or joined to the linker X at the second end of spacer J through a second J spacer functional group, and the spacer J′ is attached to the linker X′ at the second end of the spacer J′ through a second J′ spacer functional group.

In certain specific embodiments of structure II(a), each of R⁷, R^{7′}, R⁸, R^{8′}, R⁹, R^{9′}, R¹⁰, R^{10′}, R¹¹, R^{11′}, R¹², R^{12′}, R¹⁵, R^{15′}, R¹⁶R^{16′}, n, X, and X′ are as described above for structure II(a), and each of J and J′ is J1 (4,4′-diisothiocyanostilbene-2,2′-disulfonic acid (DIDS)) and the compound has the following structure II(b):

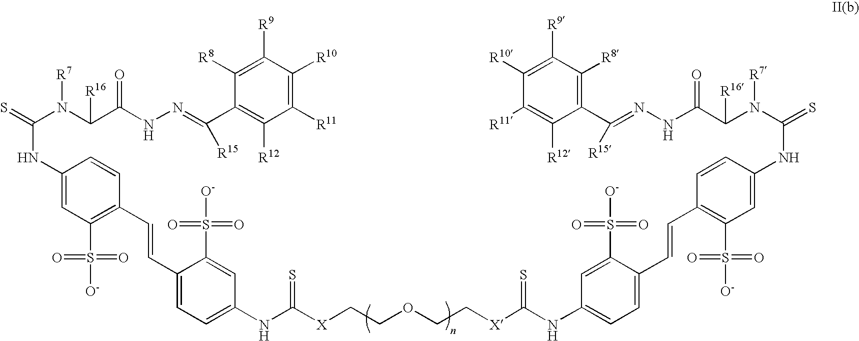

or a pharmaceutically acceptable salt, prodrug, or stereoisomer thereof wherein:

R⁷ and R^{7′} are each the same or different and independently optionally substituted phenyl, optionally substituted heteroaryl, optionally substituted quinolinyl, optionally substituted anthracenyl, or optionally substituted naphthalenyl;

R⁸, R^{8′}, R⁹, R^{9′}, R¹⁰, R^{10′}, R¹¹, R^{11′}, R¹², and R^{12′} are each the same or different and independently hydrogen, hydroxy, C₁₋₈ alkyl, C₁₋₈ alkoxy, carboxy, halo, nitro, cyano, —SO₃H, —S(═O)₂NH₂, aryl, and heteroaryl;

R¹⁵, R^{15′}, R¹⁶, and R^{16′} are each the same or different and independently hydrogen, oxo, or C₁₋₈ alkyl;

X and X′ are each the same or different linker moiety; and

n is an integer between 0 and 2,500.

In certain embodiments of compounds of structure II(b), n is any integer between 0 and 10, between 0 and 100, between 1 and 5, between 1 and 10, between 1 and 100, between 1 and 300, between 1 and 550, between 1 and 1000, between 1 and 2500, between 10 and 2500, between 10 and 2000, between 50 and 1000, between 250 and 1000, or between 450 and 1000. In more specific embodiments of structure II(b), n is any integer between 50 and 1000. In another specific embodiment, n is any integer between 200 and 300. In yet another specific embodiment, n is any integer between 450 and 550. In still another specific embodiment, n is any integer between 900 and 1000. In another specific embodiment, n is 0.

In a particular embodiment of structure II(b), R⁷ and R^{7′} are the same or different and independently unsubstituted phenyl, or substituted phenyl wherein phenyl is substituted with one or more of hydroxy, C₁₋₈ alkyl, aryl, aryloxy, —SO₃H, and C₁₋₈ alkoxy or halo wherein halo is fluoro, chloro, bromo, or iodo. In a specific embodiment, halo is chloro.

In another particular embodiment of structure II(b), R⁷ and R^{7′} are the same or different and independently 1-naphthalenyl or 2-naphthalenyl, optionally substituted with one or more of halo, hydroxy, —SH, —SO₃H, C₁₋₈ alkyl, and C₁₋₈ alkoxy; aryloxy; mono-halophenyl; di-halophenyl; mono-alkylphenyl; 2-anthracenyl; or 6-quinolinyl. In a specific embodiment, C₁₋₈ alkyl is methyl. In other specific embodiments, halo is chloro.

In another particular embodiment of structure II(b), R⁷ and R^{7′} are the same or different and independently are the same or different and independently 2-halophenyl; 4-halophenyl; -2-4-halophenyl, 4-methylphenyl; mono-(halo)naphthalenyl, di-(halo)naphthalenyl, tri-(halo)naphthalenyl, mono-(hydroxy)naphthalenyl, di-(hydroxy)naphthalenyl, tri-(hydroxy)naphthalenyl, mono-(alkoxy)naphthalenyl, di-(alkoxy)naphthalenyl, tri-(alkoxy)naphthalenyl, mono-(aryloxy)naphthalenyl, di-(aryloxy)naphthalenyl, mono-(alkyl)naphthalenyl, di-(alkyl)naphthalenyl, tri-(alkyl)naphthalenyl, mono-(hydroxy)-naphthalene-sulfonic acid, mono-(hydroxy)-naphthalene-disulfonic acid, mono(halo)-mono(hydroxy)naphthalenyl; di(halo)-mono (hydroxy)naphthalenyl; mono(halo)-di(hydroxy)naphthalenyl; di(halo)-di(hydroxy)naphthalenyl; mono-(alkyl)-mono-(alkoxy)-naphthalenyl, mono-(alkyl)-di-(alkoxy)-naphthalenyl, mono-(halo)phenyl, di-(halo)phenyl, tri-(halo) phenyl, mono-(hydroxy)phenyl, di-(hydroxy)phenyl, tri-(hydroxy)phenyl, mono-(alkoxy)phenyl, di-(alkoxy)phenyl, tri-(alkoxy)phenyl, mono-(aryloxy)phenyl, di-(aryloxy)phenyl, mono-(alkyl)phenyl, di-(alkyl)phenyl, tri-(alkyl)phenyl, mono-(hydroxy)-phenyl-sulfonic acid, mono-(hydroxy)-phenyl-disulfonic acid, mono(halo)-mono(hydroxy)phenyl, di(halo)-mono(hydroxy)phenyl, mono(halo)-di(hydroxy)phenyl, di(halo)-di(hydroxy)phenyl, mono-(alkyl)-mono-(alkoxy)-phenyl, or mono-(alkyl)-di-(alkoxy)-phenyl wherein halo is fluoro, chloro, bromo, or iodo. In a particular embodiment, halo is chloro.

In another specific embodiment of structure II(b), R⁷ and R^{7′} are the same or different and independently substituted phenyl wherein phenyl is substituted with methyl or chloro.

In yet another embodiment of structure II(b), R⁷ and R^{7′} are the same or different and independently quinolinyl or anthracenyl, optionally substituted with one or more of halo, hydroxy, C₁₋₈ alkyl, or C₁₋₈ alkoxy. In a specific embodiment, C₁₋₈ alkyl is methyl. In other specific embodiments, halo is chloro.

In still another embodiment of structure II(b), R⁷ and R^{7′} are the same or different and independently 2-naphthalenyl or 1-naphthalenyl, optionally substituted with one or more of halo, hydroxy, —SH, —SO₃H, C₁₋₈ alkyl, aryl, aryloxy, or C₁₋₈ alkoxy.

In certain embodiments of structure II(b), R⁷ and R^{7′} are the same or different and independently mono-(halo)naphthalenyl; di-(halo)naphthalenyl; tri-(halo)naphthalenyl; mono-(hydroxy)naphthalenyl; di-(hydroxy)naphthalenyl; tri-(hydroxy)naphthalenyl; mono-(alkoxy)naphthalenyl; di-(alkoxy)naphthalenyl; tri-(alkoxy)naphthalenyl; mono-(aryloxy)naphthalenyl; di-(aryloxy)naphthalenyl; mono-(alkyl)naphthalenyl; di-(alkyl)naphthalenyl; tri-(alkyl)naphthalenyl; mono-(hydroxy)-naphthalene-sulfonic acid; mono-(hydroxy)-naphthalene-disulfonic acid; mono(halo)-mono(hydroxy)naphthalenyl; di(halo)-mono(hydroxy)naphthalenyl; mono(halo)-di(hydroxy)naphthalenyl; di(halo)-di(hydroxy)naphthalenyl; mono-(alkyl)-mono-(alkoxy)-naphthalenyl; or mono-(alkyl)-di-(alkoxy)-naphthalenyl, wherein halo is fluoro, chloro, bromo, or iodo. In a specific embodiment, halo is chloro.

In yet other specific embodiments of structure II(b), R⁷ and R^{7′} are the same or different and independently 2-naphthalenyl, 2-chlorophenyl, 4-chlorophenyl, 2,4-chlorophenyl, 4-methylphenyl, 2-anthracenyl, or 6-quinolinyl.

In other particular embodiments of structure II(b), R⁷ and R^{7′} are the same or different and independently quinolinyl or anthracenyl, optionally substituted with one or more of halo, hydroxy, C₁₋₈ alkyl, or C₁₋₈ alkoxy.

In another particular embodiment of structure II(b) as described above, R⁸, R^{8′}, R⁹, R^{9′}, R¹⁰, R^{10′}, R¹¹, R^{11′}, R¹², and R^{12′} are the same or different and independently hydrogen, hydroxy, halo, carboxy, C₁₋₈ alkyl, or C₁₋₈ alkoxy.

In another particular embodiment of structure II(b), R⁸, R⁹, R¹⁰, R¹¹, and R¹² are each the same or different and independently selected from hydrogen, hydroxy, halo, carboxy, C₁₋₈ alkyl, or C₁₋₈ alkoxy, such that the phenyl group to which R⁸, R⁹, R¹⁰, R¹¹, and R¹² are attached is substituted with one, two, or three halo; one or two carboxy; one, two, or three hydroxy; one or two halo and one, two, or three hydroxy; one or two halo, one or two hydroxy, and one C₁₋₈ alkoxy; one or two halo, one hydroxy, and one or two C₁₋₈ alkoxy; or one halo, one or two hydroxy, and one or two C₁₋₈ alkoxy.

In another particular embodiment of structure II(b), R^{8′}, R^{9′}, R^{10′}, R^{11′}, and R^{12′} are each the same or different and independently selected from hydrogen, hydroxy, halo, carboxy, C₁₋₈ alkyl, or C₁₋₈ alkoxy, such that the phenyl group to which R^{8′}, R^{9′}, R^{10′}, R^{11′}, and R^{12′} are attached is substituted with one, two, or three halo; one or two carboxy; one, two, or three hydroxy; one or two halo and one, two, or three hydroxy; one or two halo, one or two hydroxy, and one C₁₋₈ alkoxy; one or two halo, one hydroxy, and one or two C₁₋₈ alkoxy; or one halo, one or two hydroxy, and one or two C₁₋₈ alkoxy.

In yet other specific embodiments of structure II(b), R⁸, R⁹, R¹⁰, R¹, and R¹² are each the same or different and independently selected from hydrogen, hydroxy, halo, carboxy, C₁₋₈ alkyl, or C₁₋₈ alkoxy, such that the phenyl group to which R⁸, R⁹, R¹⁰, R¹¹, and R¹² is attached is substituted with di(hydroxy); mono-(halo)-mono-(hydroxy); mono-(halo)-di-(hydroxy) mono-(halo)-tri-(hydroxy); di(halo)-mono-(hydroxy); di(halo)-di-(hydroxy); di(halo)-tri-(hydroxy); mono-(halo)-mono-(hydroxy)-mono-(alkoxy);mono-(halo)-di-(hydroxy)-mono-(alkoxy); mono-(halo)-mono-(hydroxy)-di-(alkoxy); mono-(halo)-di-(hydroxy)-di-(alkoxy); di-(halo)-mono-(hydroxy)-mono-(alkoxy); di-(halo)-di-(hydroxy)-mono-(alkoxy); or di-(halo)-mono-(hydroxy)-di-(alkoxy). In specific embodiments, halo is bromo. In other specific embodiments, alkoxy is methoxy.

In yet other specific embodiments of structure II(b), R^{8′}, R^{9′}, R^{10′}, R^{11′}, and R^{12′} are each the same or different and independently selected from hydrogen, hydroxy, halo, carboxy, C₁₋₈ alkyl, or C₁₋₈ alkoxy, such that the phenyl group to which R^{8′}, R^{9′}, R^{10′}, R^{11′}, and R^{12′} are attached is substituted with di(hydroxy); mono-(halo)-mono-(hydroxy); mono-(halo)-di-(hydroxy) mono-(halo)-tri-(hydroxy); di(halo)-mono-(hydroxy); di(halo)-di-(hydroxy); di(halo)-tri-(hydroxy); mono-(halo)-mono-(hydroxy)-mono-(alkoxy);mono-(halo)-di-(hydroxy)-mono-(alkoxy); mono-(halo)-mono-(hydroxy)-di-(alkoxy); mono-(halo)-di-(hydroxy)-di-(alkoxy); di-(halo)-mono-(hydroxy)-mono-(alkoxy); di-(halo)-di-(hydroxy)-mono-(alkoxy); or di-(halo)-mono-(hydroxy)-di-(alkoxy). In specific embodiments, halo is bromo. In other specific embodiments, alkoxy is methoxy.

In certain specific embodiments of structure II(b), R⁸, R⁹, R¹⁰, R¹¹, and R¹² are each the same or different and independently selected from hydrogen, hydroxy, halo, carboxy, C₁₋₈ alkyl, or C₁₋₈ alkoxy, such that the phenyl group to which R⁸, R⁹, R¹⁰, R¹¹, and R¹² are attached is 2-, 3-, or 4-halophenyl; 3,5-dihalophenyl; 2-, 3-, or 4-hydroxyphenyl; 2,4-dihydroxyphenyl; 3,5-dihalo-2,4,6-trihydroxyphenyl; 3,5-dihalo-2,4-dihydroxyphenyl; 3,5-dihalo-4-hydroxyphenyl; 3-halo-4-hydroxyphenyl; 3,5-dihalo-2-hydroxy-4-methoxyphenyl; or 4-carboxyphenyl. In a more specific embodiment, the halo is bromo.

In other certain specific embodiments structure II(b), R^{8′}, R^{9′}, R^{10′}, R^{11′}, and R^{12′} are each the same or different and independently selected from hydrogen, hydroxy, halo, carboxy, C₁₋₈ alkyl, or C₁₋₈ alkoxy, such that the phenyl group to which R^{8′}, R^{9′}, R^{10′}, R^{11′}, and R^{12′} are attached is 2-, 3-, or 4-halophenyl; 3,5-dihalophenyl; 2-, 3-, or 4-hydroxyphenyl; 2,4-dihydroxyphenyl; 3,5-dihalo-2,4,6-trihydroxyphenyl, 3,5-dihalo-2,4-dihydroxyphenyl; 3,5-dihalo-4-hydroxyphenyl; 3-halo-4-hydroxyphenyl; 3,5-dihalo-2-hydroxy-4-methoxyphenyl; or 4-carboxyphenyl, wherein halo is fluoro, chloro, bromo, or iodo. In a more specific embodiment, the halo is bromo.

In a more specific embodiment of structure II(b), each of R⁹ and R¹¹ is halo and each of R¹⁰ and R¹² is hydroxy. In another specific embodiment, each of R⁹ and R¹¹ is halo and R¹⁰ is hydroxy. In still another specific embodiment, each of R⁹ and R¹¹ is bromo, and each of R¹⁰ and R¹² is hydroxy. In yet another specific embodiment, each of R⁹ and R¹¹ is bromo, R¹⁰ is hydroxy, and R¹² is hydrogen. In other embodiments, each of R^{9′} and R^{11′} is halo and each of R^{10′} and R^{12′} is hydroxy. In still other specific embodiments, each of R^{9′} and R^{11′} is halo and R^{10′} is hydroxy. In another particular embodiment, each of R^{9′} and R^{11′} is bromo, and each of R^{10′} and R^{12′} is hydroxy. In still another particular embodiment, each of R^{9′} and R^{11′} is bromo, R^{10′} is hydroxy, and R^{12′} is hydrogen. In other specific embodiments, R⁸ and R^{8′} are each hydrogen.

In certain specific embodiments of structure II(b), each of R⁹, R^{9′}, R¹¹ and R^{11′} is halo and each of R¹⁰, R^{10′}, R¹², and R^{12′} is hydroxy. In other specific embodiments, R⁸ and R^{8′} are each hydrogen.

In other specific embodiments of structure 11(b), each of R⁹, R^{9′}, R¹¹, and R^{11′} is halo and each of R¹⁰ and R^{10′} is hydroxy. In other specific embodiments, R⁸ and R^{8′} are each hydrogen.

In yet more specific embodiments of structure II(b), each of R⁹, R^{9′}, R¹¹, and R^{11′} is bromo, and each of R¹⁰, R^{10′}, R¹², and R^{12′} is hydroxy. In other specific embodiments, R⁸ and R^{8′} are each hydrogen.

In yet more specific embodiments of structure II(b), each of R⁹, R^{9′}, R¹¹, and R^{11′} is bromo, each of R¹⁰ and R^{10′} is hydroxy, and each of R¹² and R^{12′} is hydrogen. In other specific embodiments, R⁸ and R^{8′} are each hydrogen.

In yet other specific embodiments of structure II(b), R¹⁵, R^{15′}, R¹⁶, and R^{16′} are each the same or different and independently hydrogen or methyl. In another specific embodiment, R¹⁵, R^{15′}, R¹⁶, and R^{16′} are each hydrogen. In still another specific embodiment, each of R¹⁶, and R^{16′} is the same or different and independently hydrogen or oxo.

In other more specific embodiments of structure II(b), R¹⁵, R^{15′}, R¹⁶, and R^{16′} are the same or different and independently hydrogen or methyl. In still another specific embodiment, each of R¹⁶ and R^{16′} is oxo. In such embodiments, R⁷ and R^{7′} are each the same or different and independently phenyl substituted with at least one chloro or methyl; 1-naphthalenyl; 2-naphthalenyl; 6-quinolinyl; or 2-anthracenyl. In specific embodiments, R⁸, R⁹, R¹⁰, R¹¹, R¹², R^{8′}, R^{9′}, R^{10′}, R^{11′}, and R^{12′} are each the same or different and independently hydrogen, halo, methoxy, hydroxyl, or carboxy; in specific embodiments, halo is bromo. In certain specific embodiments, when each of R⁹, R¹⁰, R¹¹, R¹², R^{9′}, R^{10′}, R^{11′}, and R^{12′} is not hydrogen, R⁸ and R^{8′} are each hydrogen.

With respect to the embodiments of structure II(b), the linker moieties X and X′ are each a functional group that may be used for conjugating the spacer J and spacer J′ (e.g., DIDS (see compounds having structure 11(b)), respectively, to polyethylene glycol (i.e., (—CH₂—O—CH₂—)ₙ). In certain specific embodiments, the linker X and the linker X′ are the same or different and independently —NH—, —O—, —S—. In a more specific embodiment, the linker X and the linker X′ are each —NH—.

In certain specific embodiments of structure II(a) and II(b), the compounds are sodium salts.

In yet more specific embodiments of structure II(a) and 11(b), the compounds have the following structures II(c), II(d), II(e), or II(f):

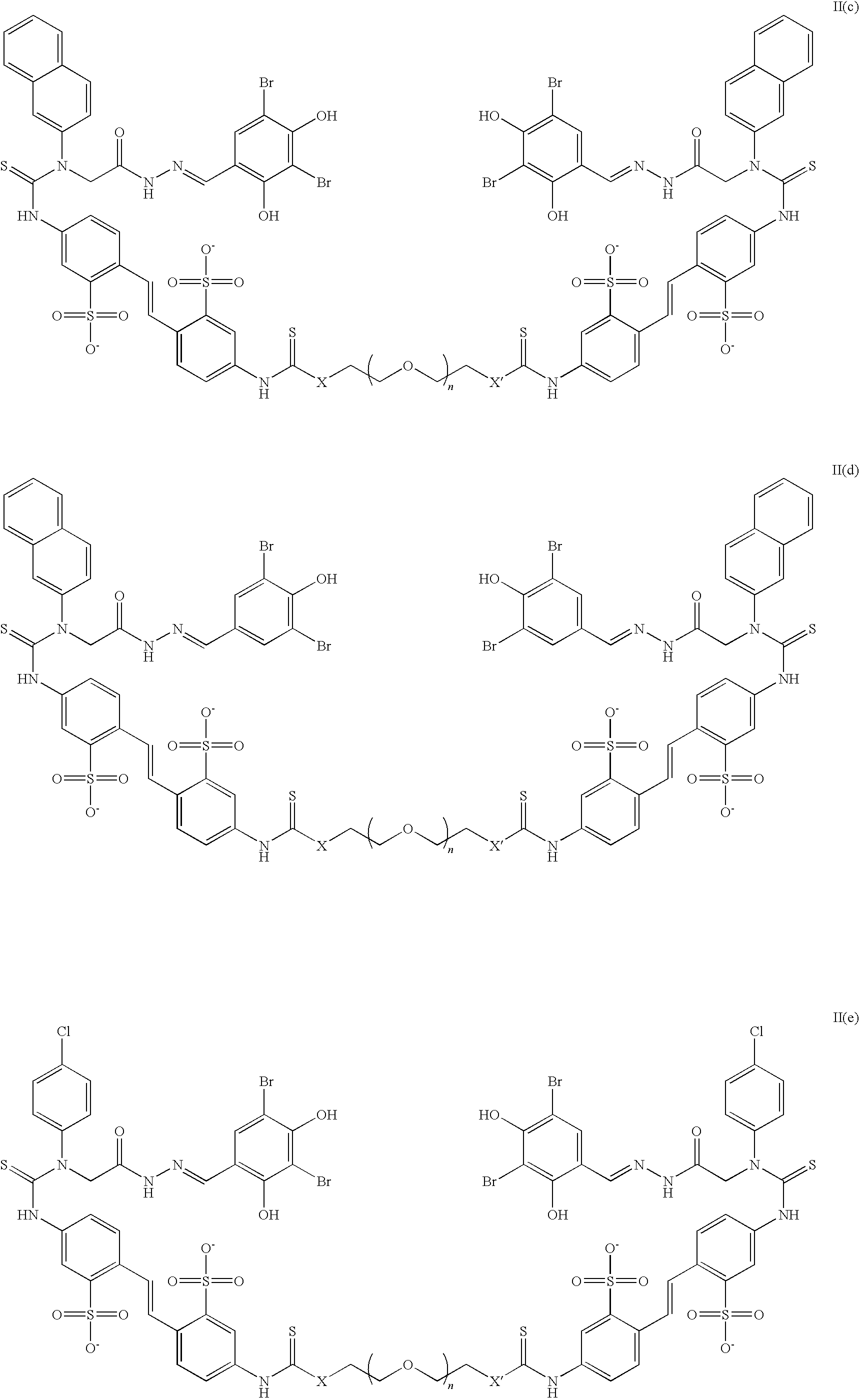

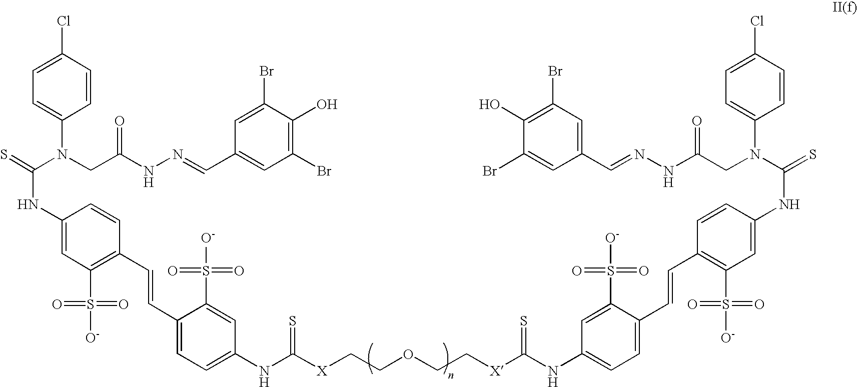

In certain specific embodiments, structures II(c), II(d), II(e), and II(f) are sodium salts.

In other specific embodiments, X and X′ are each —NH—, —O—, or —S—.

In specific embodiments, when X and X′ are each —NH—, the structures II(C), II(D), II(E), and II(F) have the specific formulae:

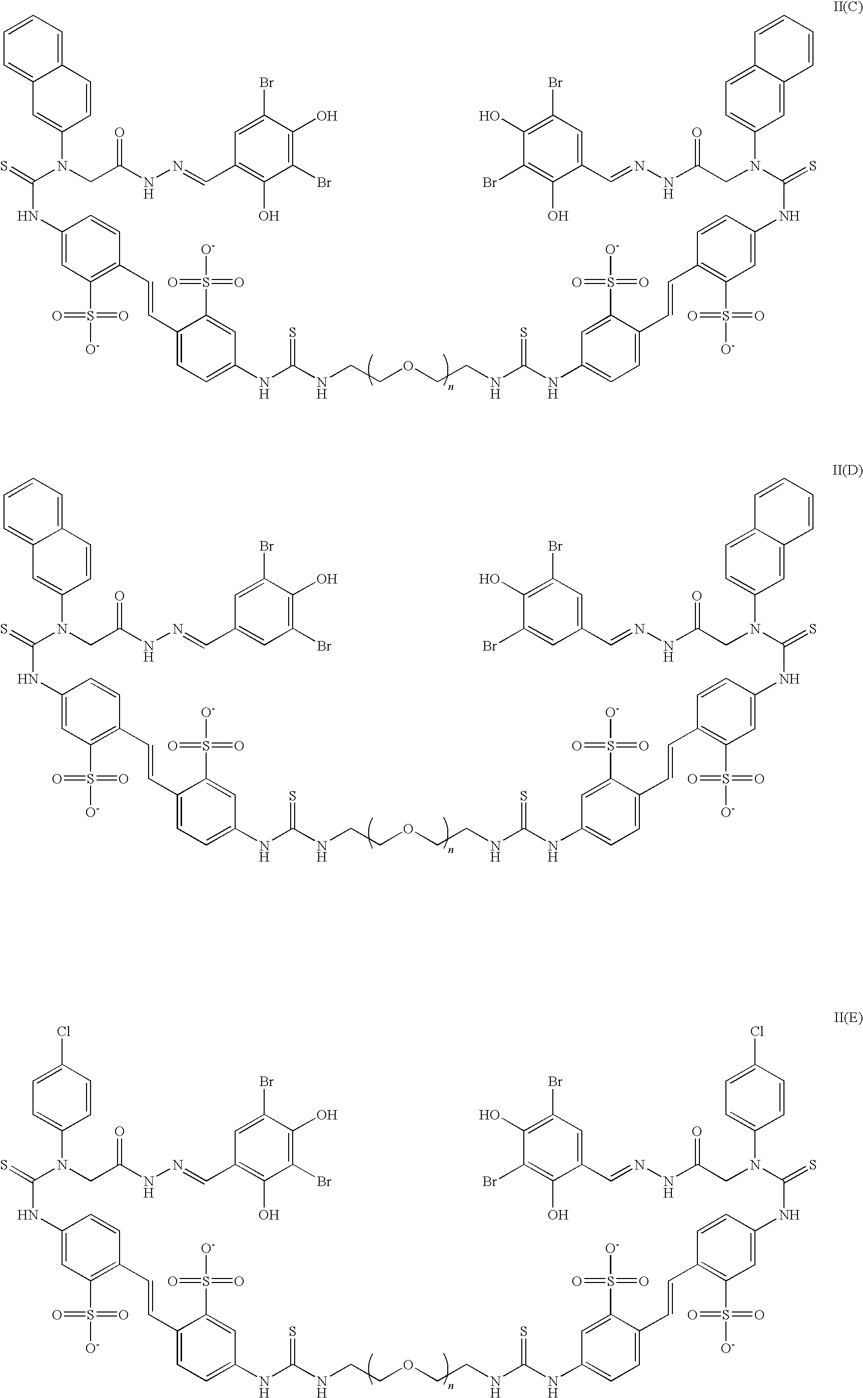

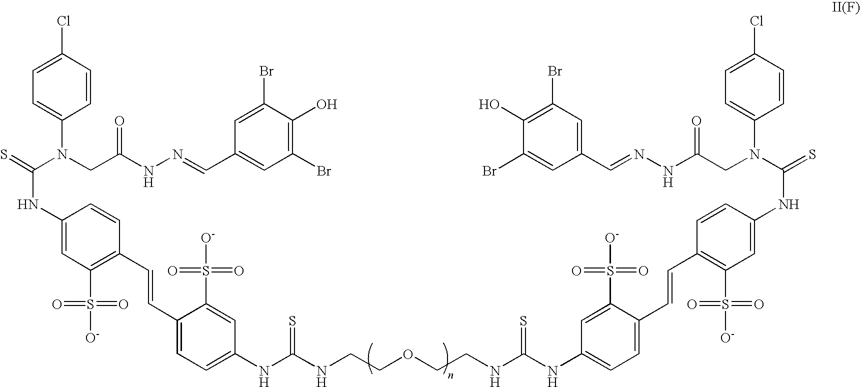

In certain specific embodiments, structures II(C), II(D), II(E), and II(F) are sodium salts.

In certain embodiments of a structure of any of formulae II(b), II(c), II(d), II(e), and II(f), II(C), II(D), II(E), and II(F)ₙ is any integer between 0 and 10, between 0 and 100, between 1 and 5, between 1 and 10, between 1 and 100, between 1 and 300, between 1 and 550, between 1 and 1000, between 1 and 2500, between 10 and 2500, between 10 and 2000, between 50 and 1000, between 250 and 1000, or between 450 and 1000. In more specific embodiments of structures II(b), II(c), II(d), II(e), and II(f), and II((C)-(F)), n is any integer between 50 and 1000. In another specific embodiment, n is any integer between 200 and 300. In yet another specific embodiment, n is any integer between 450 and 550. In still another specific embodiment, n is any integer between 900 and 1000. In another specific embodiment, n is 0.

The conjugate compounds having a structure of any one of formulae II(a), II(b), II(c), II(d), II(e), and II(f) or any substructure thereof (e.g., II(C)-(F)) are also referred to herein as divalent glycine hydrazide-PEG conjugate compounds (or divalent glycine hydrazide-PEG conjugates).

In certain specific embodiments the divalent glycine hydrazide PEG conjugate compound has one of the following structures:

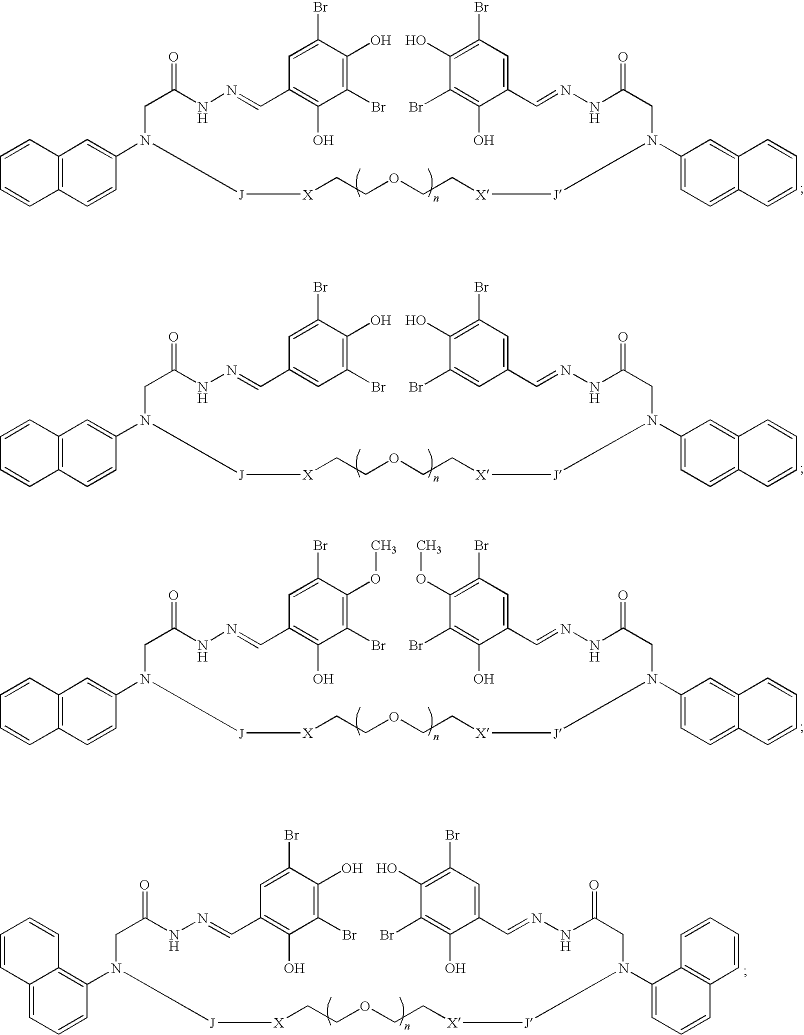

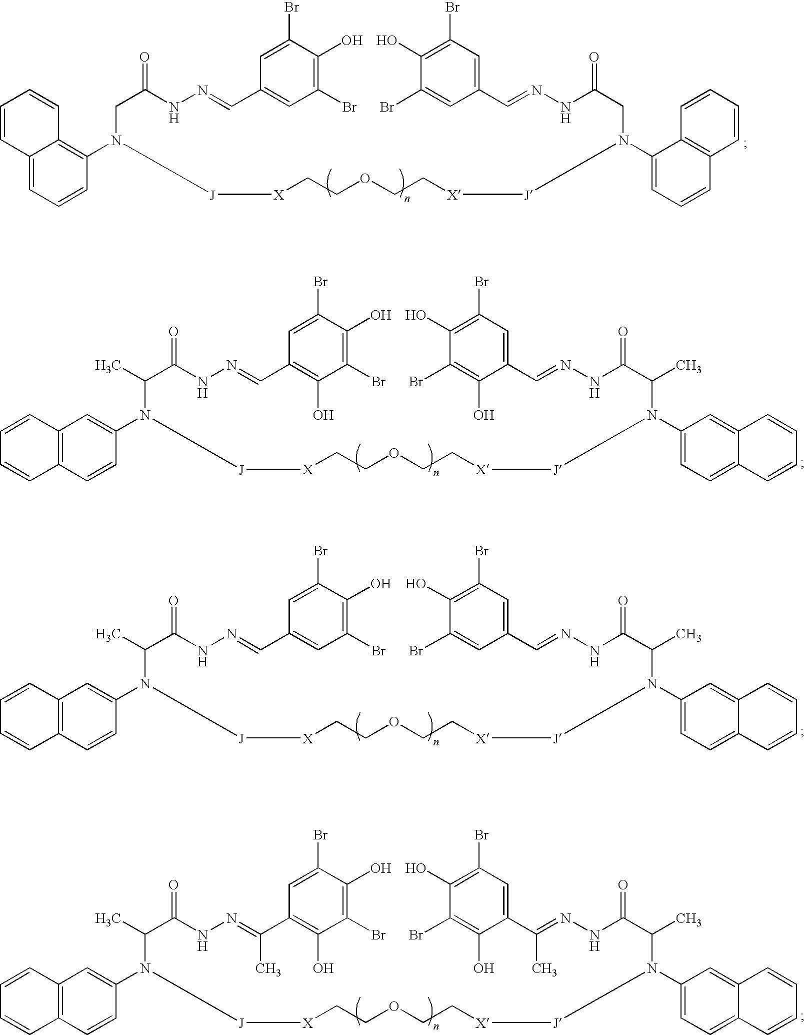

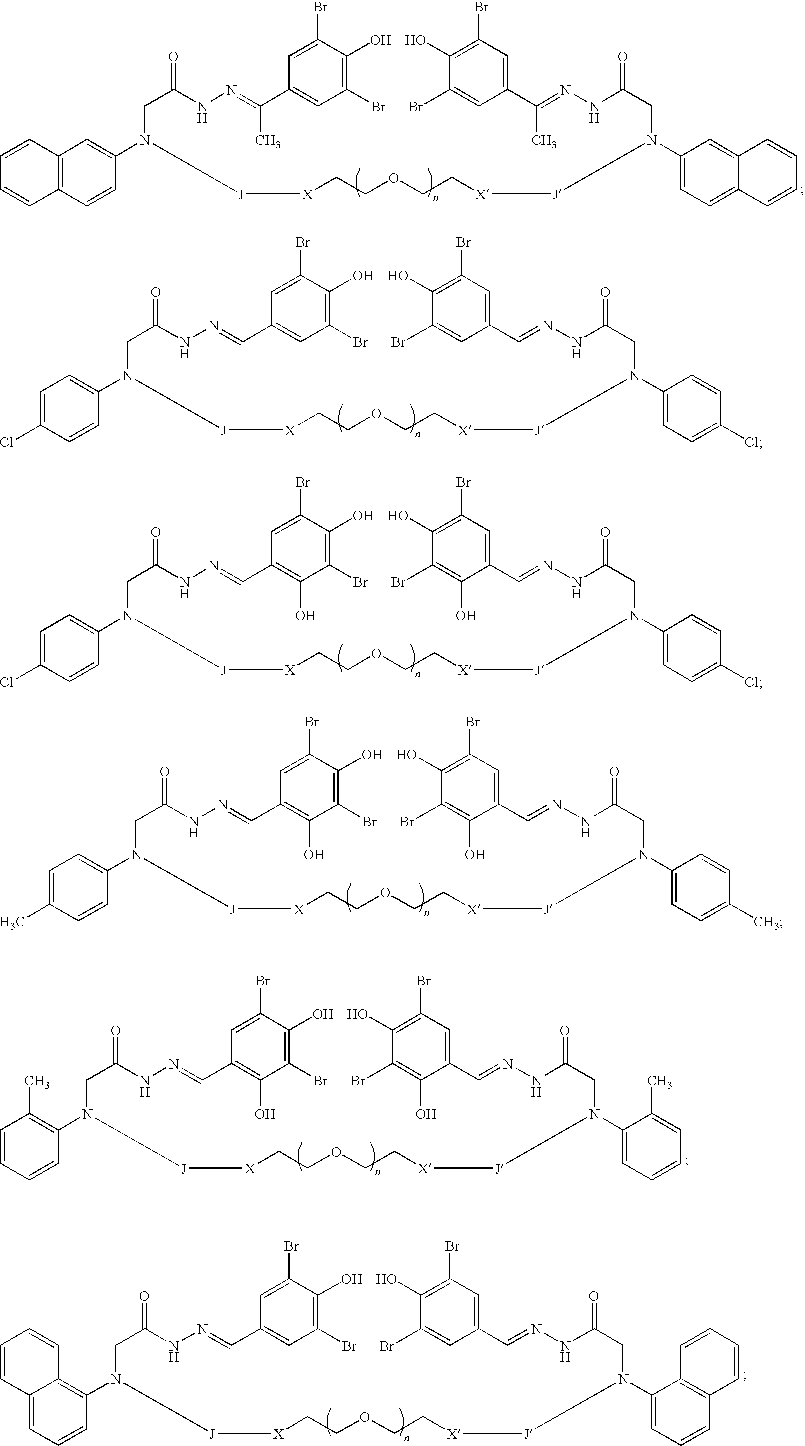

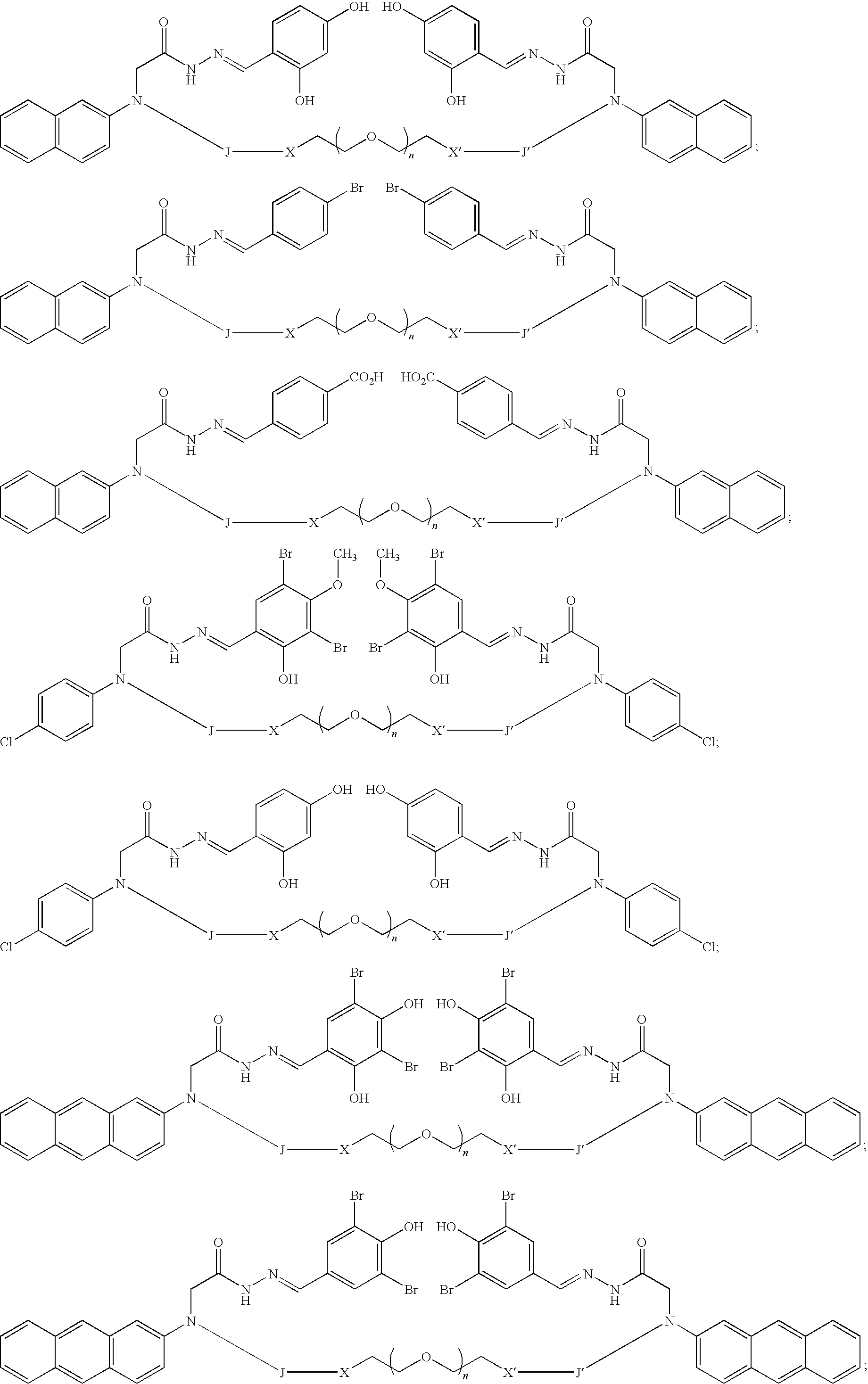

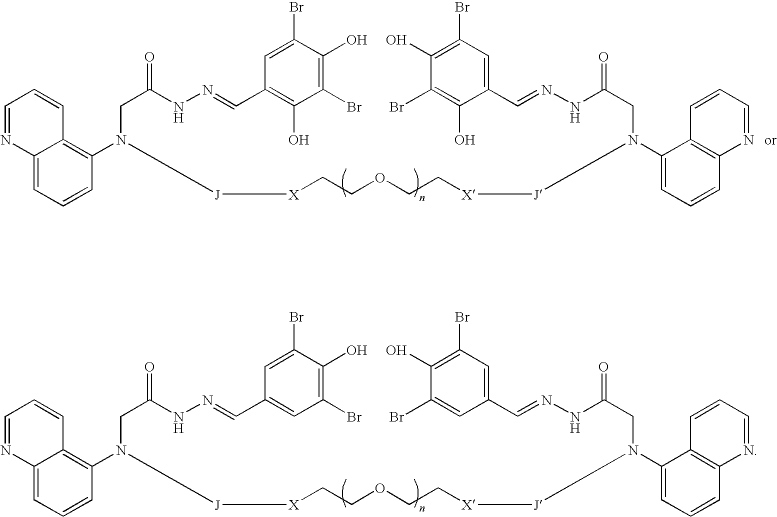

or a pharmaceutically acceptable salt, prodrug, or stereoisomer thereof, wherein J and J′ are each independently any one of spacer J1-J29 as described herein, and wherein X and X′ are each independently —NH—, —O—, or —S—. In certain embodiments, each of J and J′ is J1 (4,4′-diisothiocyanostilbene-2,2′-disulfonic acid (DIDS)). In other specific embodiments, X and X′ are each —NH—.

### Chemistry Definitions

Certain chemical groups named herein are preceded by a shorthand notation indicating the total number of carbon atoms that are to be found in the indicated chemical group. For example; C₁-C₈ alkyl describes an alkyl group, as defined below, having a total of 1 to 8 carbon atoms, and C₃-C₁₂ cycloalkyl describes a cycloalkyl group, as defined below, having a total of 3 to 12 carbon atoms. The total number of carbons in the shorthand notation does not include carbons that may exist in substituents of the group described. In addition to the foregoing, as used herein, unless specified to the contrary, the following terms have the meaning indicated.

“Alkyl” means a straight chain or branched, noncyclic or cyclic, unsaturated or saturated aliphatic hydrocarbon containing from 1 to 18 carbon atoms, while the term “C₁₋₈ alkyl” has the same meaning as alkyl but contain from 1 to 8 carbon atoms. Representative saturated straight chain alkyls include methyl, ethyl, n-propyl, n-butyl, n-pentyl, n-hexyl, and the like, while saturated branched alkyls include isopropyl, sec-butyl, isobutyl, tert-butyl, heptyl, n-octyl, isopentyl, 2-ethylhexyl and the like. Representative saturated cyclic alkyls include cyclopropyl, cyclobutyl, cyclopentyl, cyclohexyl, —CH₂cyclopropyl, —CH₂cyclobutyl, —CH₂cyclopentyl, —CH₂cyclohexyl, and the like; unsaturated cyclic alkyls include cyclopentenyl and cyclohexenyl, and the like. Cyclic alkyls, also referred to as “homocyclic rings,” include di- and poly-homocyclic rings such as decalin and adamantyl. Unsaturated alkyls contain at least one double or triple bond between adjacent carbon atoms (referred to as an “alkenyl” or “alkynyl,” respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; representative straight chain and branched alkynyls include acetylenyl, propynyl, 1-butynyl, 2-butynyl, 1-pentynyl, 2-pentynyl, 3-methyl-I butynyl, and the like.

Within the context of the compounds described herein, the terms alkyl, aryl, arylalkyl, heterocycle, homocycle, and heterocycloalkyl are taken to comprise unsubstituted alkyl and substituted alkyl, unsubstituted aryl and substituted aryl, unsubstituted arylalkyl and substituted arylalkyl, unsubstituted heterocycle and substituted heterocycle, unsubstituted homocycle and substituted homocycle, unsubstituted heterocycloalkyl and substituted heterocycloalkyl, respectively, as defined herein, unless otherwise specified.

As used herein, the term “substituted” in the context of alkyl, aryl, arylalkyl, heterocycle, and heterocycloalkyl means that at least one hydrogen atom of the alkyl, aryl, arylalkyl, heterocycle or heterocycloalkyl moiety is replaced with a substituent. In the instance of an oxo substituent (“═O”) two hydrogen atoms are replaced. A “substituent” as used within the context of this disclosure includes oxo, halogen, hydroxy, cyano, nitro, amino, alkylamino, dialkylamino, alkyl, alkoxy, thioalkyl, haloalkyl, substituted alkyl, heteroalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocycloalkyl, substituted heterocycloalkyl, —NRₐR_{b}, —NRₐC(═O)R_{b}, —NRₐC(═O)NRₐR_{b}, —NRₐC(═O)OR_{b}—NRₐS(═O)₂R_{b}, —ORₐ, —C(═O)Rₐ—C(═O)ORₐ, —C(═O)NRₐR_{b}, —OCH₂C(═O)NRₐR_{b}, —OC(═O)NRₐR_{b}, —SH, —SRₐ, —SORₐ, —S(═O)₂NRₐR_{b}, —S(═O)₂Rₐ, —SRₐC(═O)NRₐR_{b}, —OS(═O)₂Rₐ and —S(═O)₂ORₐ (also written as —SO₃Rₐ), wherein Rₐ and R_{b} are the same or different and independently hydrogen, alkyl, haloalkyl, substituted alkyl, alkoxy, aryl, substituted aryl, arylalkyl, substituted arylalkyl, arylalkoxy, heteroaryl, substituted heteroaryl, heteroarylalkyl, substituted heteroarylalkyl, heterocycle, substituted heterocycle, heterocycloalkyl or substituted heterocycloalkyl. The definitions of Rₐ and R_{b} above apply to all uses of these substituents throughout the description.

Representative substituents include (but are not limited to) alkoxy (i.e., alkyl-O—, including C₁₋₈ alkoxy e.g., methoxy, ethoxy, propoxy, butoxy, pentoxy,), aryloxy (e.g., phenoxy, chlorophenoxy, tolyloxy, methoxyphenoxy, benzyloxy, alkyloxycarbonylphenoxy, alkyloxycarbonyloxy, acyloxyphenoxy), acyloxy (e.g., propionyloxy, benzoyloxy, acetoxy), carbamoyloxy, carboxy, mercapto, alkylthio, acylthio, arylthio (e.g., phenylthio, chlorophenylthio, alkylphenylthio, alkoxyphenylthio, benzylthio, alkyloxycarbonyl-phenylthio), amino (e.g., amino, mono- and di-C₁-C₃ alkanylamino, methylphenylamino, methylbenzylamino, C₁-C₃ alkanylamido, acylamino, carbamamido, ureido, guanidino, nitro and cyano). Moreover, any substituent may have from 1-5 further substituents attached thereto.

“Aryl” means an aromatic carbocyclic moiety such as phenyl or naphthyl (i.e., naphthalenyl) (1- or 2-naphthyl) or anthracenyl (e.g., 2-anthracenyl).

“Arylalkyl” (e.g., phenylalkyl) means an alkyl having at least one alkyl hydrogen atom replaced with an aryl moiety, such as —CH₂-phenyl, —CH═CH-phenyl, —C(CH₃)═CH-phenyl, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are furyl, benzofuranyl, thiophenyl, benzothiophenyl, pyrrolyl, indolyl, isoindolyl, azaindolyl, pyridyl, quinolinyl (including 6-quinolinyl), isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

“Heteroarylalkyl” means an alkyl having at least one alkyl hydrogen atom replaced with a heteroaryl moiety, such as —CH₂pyridinyl, —CH₂pyrimidinyl, and the like.

“Heterocycle” (also referred to herein as a “heterocyclic ring”) means a 4- to 7-membered monocyclic, or 7- to 10-membered bicyclic, heterocyclic ring which is saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined herein. Thus, in addition to the heteroaryls listed above, heterocycles also include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The term “optionally substituted” as used in the context of an optionally substituted heterocycle (as well heteroaryl) means that at least one hydrogen atom is replaced with a substituent. In the case of a keto substituent (“—C(═O)—”) two hydrogen atoms are replaced. When substituted, one or more of the above groups are substituted. “Substituents” within the context of description herein are also described above and include halogen, hydroxy, cyano, nitro, amino, alkylamino, dialkylamino, alkyl, alkoxy, alkylthio, haloalkyl, aryl, arylalkyl, heteroaryl, heteroarylalkyl, heterocycle and heterocycloalkyl, as well as —NRₐR_{b}, —NRₐC(═O)R_{b}, —NRₐC(═O)NRₐR_{b}, —NRₐC(═O)OR_{b}—NRₐS(═O)₂R_{b}, —ORₐ, —C(═O)Rₐ—C(═O)ORₐ, —C(═O)NRₐR_{b}, —OCH₂C(═O)NRₐR_{b}, —OC(═O)NRₐR_{b}, —SH, —SRₐ, —SORₐ, —S(═O)₂NRₐR_{b}, —S(═O)₂Rₐ, —OS(═O)₂Rₐ and —S(═O)₂ORₐ. In addition, the above substituents may be further substituted with one or more of the above substituents, such that the substituent is a substituted alkyl, substituted aryl, substituted arylalkyl, substituted heterocycle or substituted heterocycloalkyl. Rₐ and R_{b} in this context may be the same or different and independently hydrogen, alkyl, haloalkyl, substituted alkyl, alkoxy, aryl, substituted aryl, arylalkyl, substituted arylalkyl, heterocycle (including heteroaryl), substituted heterocycle (including substituted heteroaryl), heterocycloalkyl, or substituted heterocycloalkyl.

“Heterocycloalkyl” means an alkyl having at least one alkyl hydrogen atom replaced with a heterocycle, such as —CH₂-morpholinyl, —CH₂CH₂piperidinyl, —CH₂azepineyl, —CH₂pirazineyl, —CH₂pyranyl, —CH₂furanyl, —CH₂pyrrolidinyl, and the like.

“Homocycle” (also referred to herein as “homocyclic ring”) means a saturated or unsaturated (but not aromatic) carbocyclic ring containing from 3-7 carbon atoms, such as cyclopropane, cyclobutane, cyclopentane, cyclohexane, cycloheptane, cyclohexene, and the like.

“Halogen” or “halo” means fluoro, chloro, bromo, and iodo.

“Haloalkyl,” which is an example of a substituted alkyl, means an alkyl having at least one hydrogen atom replaced with halogen, such as trifluoromethyl and the like.

“Haloaryl,” which is an example of a substituted aryl, means an aryl having at least one hydrogen atom replaced with halogen, such as 4-fluorophenyl and the like.

“Alkoxy” means an alkyl moiety attached through an oxygen bridge (i.e., —O-alkyl) such as methoxy, ethoxy, and the like.

“Haloalkoxy,” which is an example, of a substituted alkoxy, means an alkoxy moiety having at least one hydrogen atom replaced with halogen, such as chloromethoxy and the like.

“Alkoxydiyl” means an alkyl moiety attached through two separate oxygen bridges (i.e., —O-alkyl-O—) such as —O—CH₂—O—, —O—CH₂CH₂—O—, —O—CH₂CH₂CH₂—O—, —O—CH(CH₃)CH₂CH₂—O—, —O—CH₂C(CH₃)₂CH₂—O—, and the like.

“Alkanediyl” means a divalent alkyl from which two hydrogen atoms are taken from the same carbon atom or from different carbon atoms, such as —CH₂—, —CH₂CH₂—, —CH₂CH₂CH₂—, —CH(CH₃)CH₂CH₂—, —CH₂C(CH₃)₂CH₂—, and the like.

As used herein, “alkenylene” refers to a straight, branched or cyclic, in one embodiment straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —CH═CH—CH₂—. The term “lower alkenylene” refers to alkenylene groups having 2 to 6 carbons. In certain embodiments, alkenylene groups are lower alkenylene, including alkenylene of 3 to 4 carbon atoms.

As used herein, “alkynylene” refers to a straight, branched or cyclic, in certain embodiments straight or branched, divalent aliphatic hydrocarbon group, in one embodiment having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C—C—, —C≡C— and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

“Thioalkyl” means an alkyl moiety attached through a sulfur bridge (i.e., —S-alkyl) such as methylthio, ethylthio, and the like.

“Alkylamino” and “dialkylamino” mean one or two alkyl moieties attached through a nitrogen bridge (i.e., —N-alkyl) such as methylamino, ethylamino, dimethylamino, diethylamino, and the like.

“Carbamate” is —RₐOC(═O)NRₐR_{b}.

“Cyclic carbamate” means any carbamate moiety that is part of a ring.

“Amidyl” is —NRₐR_{b}.

“Hydroxyl” or “hydroxy” refers to the —OH radical.

“Sulfhydryl” or “thio” is —SH.

“Amino” refers to the —NH₂ radical.

“Nitro” refers to the —NO₂ radical.

“Imino” refers to the ═NH radical.

“Thioxo” refers to the ═S radical.

“Cyano” refers to the —C≡N radical.

“Sulfonamide refers to the radical —S(═O)₂NH₂.

“Isocyanate” refers to the —N═C═O radical.

“Isothiocyanate” refers to the —N═C═S radical.

“Azido” refers to the —N═N⁺═N⁻ radical.

“Carboxy” refers to the —CO₂H radical (also depicted as —C(═O)OH or COOH).

“Hydrazide” refers to the —C(═O)NRₐ—NRₐR_{b} radical.

“Oxo” refers to the ═O radical.

A polyethylene imine (PEI) monomer is a three-membered ring. Two “corners” of the molecule consist of —CH₂— linkages, and the third “corner” is a secondary amine group, ═NH.

Each of —CH₂—O—CH₂— (representing a monomeric unit of polyethylene glycol (PEG)) in any of structures of formulae I(a), I(b), I(c)-I(j), II(a), II(b), II(c), II(d), II(e), and II(f) and II((C)-(F)) described herein has a calculated molecular weight of 44 daltons. When n of any of these formulae is between 1 and 2500, the estimated molecular weight contributed by (—CH₂—O—CH₂—)n is therefore between about 0.044 kDa and about 110 kDa; when n is between 10 and 2500, the estimated molecular weight contributed by (—CH₂—O—CH₂—)n is between about 0.44 kDa and about 110 kDa; when n is between 10 and 2000, the estimated molecular weight contributed by (—CH₂—O—CH₂—)ₙ is between about 0.44 kDa and about 88 kDa; when n is between 50 and 1000, the estimated molecular weight contributed by (—CH₂—O—CH₂—)ₙ is between 2.2 kDa and 44 kDa; when n is between 250 and 1000, the estimated molecular weight contributed by (—CH₂—O—CH₂—)ₙ is between about 11 kDa and about 44 kDa; when n is between 450 and 1000, the estimated molecular weight contributed by (—CH₂—O—CH₂—)n is between about 20 kDa and about 44 kDa. When n of any of these formulae is between 200 and 300, the estimated molecular weight contributed by (—CH₂—O—CH₂—)ₙ is therefore between about 8.8 kDa and about 13 kDa; when n of any of these formulae is between 450 and 550, the estimated molecular weight contributed by (—CH₂—O—CH₂—)ₙ is therefore between about 20 kDa and about 24 kDa; and when n of any of these formulae is between 900 and 1000, the estimated molecular weight contributed by (—CH₂—O—CH₂—)ₙ is therefore between about 40 kDa and about 44 kDa. In certain specific embodiments, the estimated molecular weight contributed by (—CH₂—O—CH₂—)ₙ is 0.2, 3, 6, 10, 20, 40, or 100 kDa. In more particular embodiments, the estimated molecular weight contributed by (—CH₂—O—CH₂—)ₙ is 10, 20, or 40 kDa.

The compounds described herein may generally be used as the free acid or free base. Alternatively, the compounds may be used in the form of acid or base addition salts. Acid addition salts of the free base amino compounds may be prepared according to methods well known in the art, and may be formed from organic and inorganic acids. Suitable organic acids include (but are not limited to) maleic, fumaric, benzoic, ascorbic, succinic, methanesulfonic, acetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include (but are not limited to) hydrochloric, hydrobromic, sulfuric, phosphoric, and nitric acids. Base addition salts of the free acid compounds of the compounds described herein may also be prepared by methods well known in the art, and may be formed from organic and inorganic bases. Suitable inorganic bases included (but are not limited to) the hydroxide or other salt of sodium, potassium, lithium, ammonium, calcium, magnesium, iron, zinc, copper, manganese, aluminum, and the like, and organic bases such as substituted ammonium salts. Thus, the term “pharmaceutically acceptable salt” of structure I, I(a) and structure II, and II(a), as well as any and all substructures and specific compounds and conjugates described herein is intended to encompass any and all pharmaceutically suitable salt forms.

Structures I, I(a), II and II(a) and substructures thereof as well as J and J′ may sometimes be depicted as an anionic species. For instance, the compounds may be depicted as the sulfonic acid (SO₃⁻) anion. One of ordinary skill in the art will recognize that the compounds exist with an equimolar ratio of cation. For instance, the compounds described herein can exist in the fully protonated form, or in the form of a salt such as sodium, potassium, ammonium or in combination with any inorganic base as described above. When more than one anionic species is depicted, each anionic species may independently exist as either the protonated species or as the salt species. In some specific embodiments, the compounds described herein exist as the sodium salt.

Also contemplated are prodrugs of any of the compound conjugates described herein. Prodrugs are any covalently bonded carriers that release a conjugate compound of structure I, I(a), II, or II(a), as well as any of the substructures herein, in vivo when such prodrug is administered to a subject. Prodrugs are generally prepared by modifying functional groups in a way such that the modification is cleaved, either by routine manipulation or by an in vivo process, yielding the parent compound. Prodrugs include, for example, conjugate compounds described herein when, for example, hydroxy or amine groups are bonded to any group that, when administered to a subject, is cleaved to form the hydroxy or amine groups. Thus, representative examples of prodrugs include (but are not limited to) acetate, formate and benzoate derivatives of alcohol and amine functional groups of the compounds of structure I, I(a), II, or II(a), as well as any of the substructures herein. Further, in the case of a carboxylic acid (—COOH), esters may be employed, such as methyl esters, ethyl esters, and the like. Prodrug chemistry is conventional to and routinely practiced by a person having ordinary skill in the art.

Prodrugs are typically rapidly transformed in vivo to yield the parent compound (I.e., a compound conjugate of formula I or I(a) or subformulae I(b)-I(j) or of formulae II or II(a) or subformulae II(b), II(c), II(d), II(e), and II(f)), and II((C)-(F)), for example, by hydrolysis in blood. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam)). A discussion of prodrugs is provided in Higuchi, T., et al., “Pro-drugs as Novel Delivery Systems,” A.C.S. Symposium Series, Vol. 14, and in Bioreversible Carriers in Drug Design, Ed. Edward B. Roche, American Pharmaceutical Association and Pergamon Press, 1987, both of which are incorporated in full by reference herein.

With regard to stereoisomers, the conjugate compounds of structure I, I(a), II, or II(a), as well as any substructure described herein, may have one or more chiral centers and may occur in any isomeric form, including racemates, racemic mixtures, and as individual enantiomers or diastereomers. In addition, the conjugate compounds of structure I, I(a), II or II(a), as well as any substructure thereof, may contain olefinic double bonds or other centers of geometric asymmetry, and unless specifically indicated otherwise, include both E and Z geometric isomers (e.g., cis or trans). Likewise, all possible isomers, as well as their racemic and optically pure forms, and all tautomeric forms are also intended to be included. A tautomer refers to a proton shift from one atom of a molecule to another atom of the same molecule. All such isomeric forms of the compounds are included and contemplated, as well as mixtures thereof. Furthermore, some of the crystalline forms of any compound described herein may exist as polymorphs, which are also included and contemplated by the present disclosure. In addition, some of the compounds may form solvates with water or other organic solvents. Such solvates are similarly included within the scope of compounds and compositions described herein.

### Compound Synthesis

In general, the compounds used in the reactions described herein may be made according to organic synthesis techniques known to those skilled in this art, starting from commercially available chemicals and/or from compounds described in the chemical literature. “Commercially available chemicals” may be obtained from standard commercial sources including Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hanover, Germany), Spectrum Quality Product, Inc. (New Brunswick, N.J.), TCI America (Portland Oreg.), Trans World Chemicals, Inc. (Rockville Md.), and Wako Chemicals USA, Inc. (Richmond Va.).

Methods known to one of ordinary skill in the art may be identified through various reference books and databases. Suitable reference books and treatises that detail the synthesis of reactants useful in the preparation of compounds and compound conjugates described herein, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Modem Synthetic Reactions,” 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of conjugate compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modem Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J.C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the hydrazide compounds and conjugate compounds described herein is P. H. Stahl & C. G. Wermuth “Handbook of Pharmaceutical Salts”, Verlag Helvetica Chimica Acta, Zurich, 2002.

With respect to methods for synthesizing malonic and glycine hydrazide compounds, also see U.S. Pat. No. 7,414,037, Muanprasat et al., J. Gen. Physiol. 124:125-37 (2004), and Sonawane et al., FASEB J. 20:130-132 (2006)). Additional detail describing synthesis of the divalent malonic hydrazide compounds is provided in Example 1.

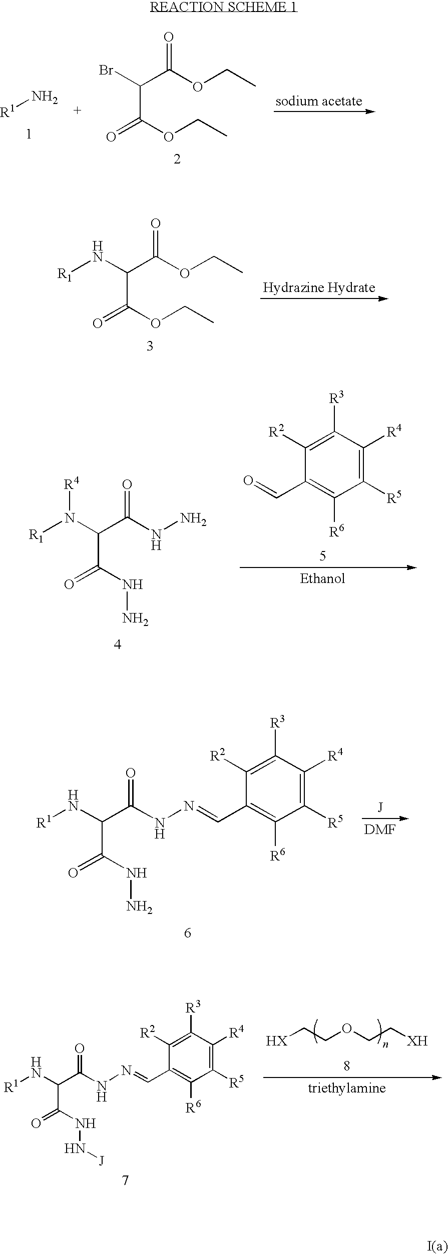

In general, conjugate compounds of formula I and I(a) can be prepared according to Reaction Scheme 1. Referring to Reaction Scheme 1, reactant 1 is combined with diethyl bromomalonate (2), each at 10 mmol. The resulting reaction mixture is then stirred at an elevated temperature for about 8 hours. Upon cooling, the solid material is filtered and recrystallized from hexane to yield the compound of formula 3. A solution of 3 in ethanol is then refluxed with 12 mmol hydrazine hydrate for about 10 hours. The solvent and excess reagent are then distilled under vacuum. The product is then recrystallized from ethanol to yield the compound of formula 4. The compound of formula 4 is then combined with aldehyde 5 in ethanol and then refluxed for about 3 hours to yield the desired product 6. The compound of formula 6 is then combined with J (any of J1-J29) in DMF to yield the spacer-linked compound 7. Compound 7 is then conjugated to a polyethylene glycol moiety of formula 8 to yield compounds of formula I(a).

One skilled in the art will recognize that when any one of R¹, R², R³, R⁴, R⁵, and R⁶ are not the same as R^{1′}, R^{2′}, R^{3′}, R^{4′}, R^{5′}, and R^{6′}, respectively, compounds of formula I(a) can be prepared by first reacting a compound of formula 7 with 8 in a 1:1 ratio followed by reaction of the resultant product with an excess of a different compound of formula 7.

Alternatively, the second end of spacer J may be attached first to a polyethylene glycol moiety 8. The resulting compound can then be reacted with compounds of formula 6 to obtain compound of formula I(a).

A person skilled in the art will readily understand that the valency of a spacer J described herein adjusts to retain stability of the compound. For example, where I(a) attaches to J via an isothiocyanate (as in J1 for example), the nitrogen atom of the isothiocyanate will add a hydrogen to retain stability.

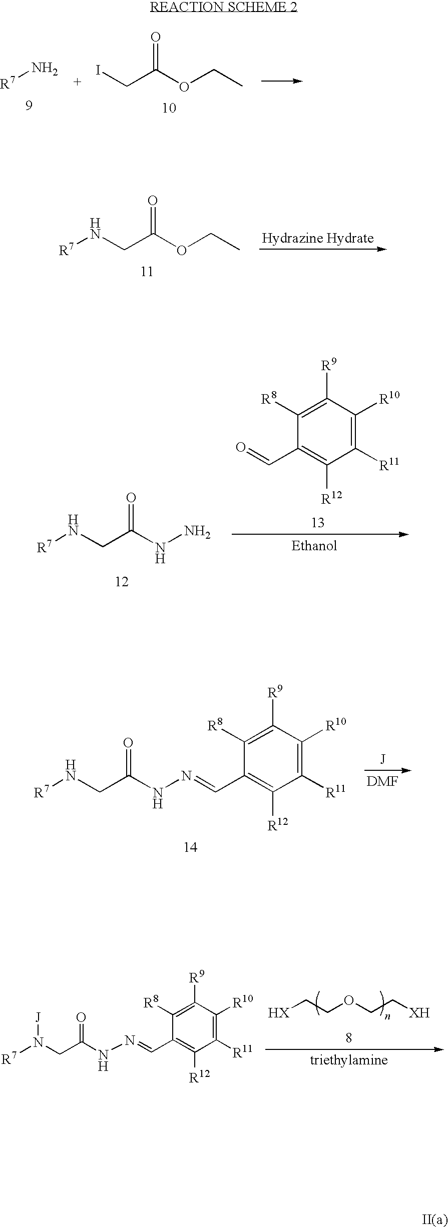

In general, compounds of formula II and II(a) are prepared according to Reaction Scheme 2. Referring to Reaction Scheme 2, compounds 9 and 10 are combined to form 11. The compound of formula II is solubilized in ethanol and refluxed with 12 mmol hydrazine hydrate for about 10 hours. The solvent and excess reagent are then distilled under vacuum. The product is recrystallized from ethanol to yield the compound of formula 12. The compound of formula 12 is then combined with aldehyde 13 in ethanol and then refluxed for about 3 hours to yield the desired compound of Formula 14. Treatment of 14 with J in DMF yields compound 15. Compounds of formula II(a) are then obtained by reaction of a polyethylene glycol moiety with 15.

As in Reaction Scheme 1, one skilled in the art will also recognize that when any one of R⁷, R⁸, R⁹, R¹⁰, R¹¹, and R¹² is not the same as R^{7′}, R^{8′}, R^{9′}, R^{10′}, R^{11′}, and R^{12′}, respectively, compounds of formula II(a) can be prepared by first reacting a compound of formula 15 with 8 in a 1:1 ratio followed by reaction of the resultant product with an excess of a different compound of formula 15.

Alternatively, the second end of spacer J may be attached first to a polyethylene glycol moiety 8. The resulting compound can then be reacted with compounds of formula 14 to obtain compound of formula I(a).

Preparation of Hydrazide-Polyethylene Glycol (Peg) Conjugates May be performed according to methods practiced in the art and described herein. Monovalent and divalent PEG conjugates may be synthesized by reaction of the corresponding bisamino and monoamino PEGs with a 5-fold molar excess of a malonic hydradize or glycine hydrazide compound that is attached to a spacer J, such as MalH-DIDS, in anhydrous DMSO in presence of triethylamine as a base catalyst. Unreacted compound is removed by an amino-functionalized scavenger, and the PEG conjugates can be purified by methods routinely used in the art, for example, controlled precipitation and combinations of gel filtration, dialysis, ion exchange chromatography, and preparative HPLC.

### Methods for Characterizing and Using the Divalent Hydrazide-PEG Conjugate Compounds

The divalent hydrazide-polymer conjugate compounds having a structure of either formula I or II and the divalent hydrazide-PEG conjugate compounds having a structure of formula I(a) or subformulae I(b), I(c)-I(j) or of formula II(a) or subformulae II(b), II(c), II(d), II(e), and II(f) and II((C)-(F)) described herein are capable of blocking or impeding the CFTR pore or channel and inhibiting ion transport by CFTR located in the outer cell membrane of a cell. Also provided herein are methods of inhibiting ion transport by CFTR , which comprises contacting a cell that has CFTR in the outer membrane with any one of the conjugate compounds described herein, under conditions and for a time sufficient for the CFTR and the compound to interact.

Divalent hydrazide conjugate compounds may be identified and/or characterized by such a method of inhibiting ion transport by CFTR , performed with isolated cells in vitro. In certain embodiments, these methods may be performed using a biological sample as described herein that comprises, for example, cells obtained from a tissue, body fluid, or culture adapted cell line or other biological source as described in detail herein below. The step of contacting the cell that has CFTR in the outer membrane with the at least one compound refers to combining, mixing, or in some other manner of contacting familiar to persons skilled in the art, that permits the compound and the cell to interact such that any effect of the compound on CFTR activity can be measured according to methods described herein and routinely practiced in the art. Methods described herein for inhibiting ion transport by CFTR are understood to be performed under conditions and for a time sufficient that permit the CFTR and the compound to interact. Conditions for a particular assay include temperature, buffers (including salts, cations, media), and other components that maintain the integrity of the cell and the compound, which a person skilled in the art will be familiar and/or which can be readily determined. A person skilled in the art also readily appreciates that appropriate controls can be designed and included when performing the in vitro methods described herein.

Methods for characterizing a compound conjugate, such as determining an effective concentration to achieve a therapeutic benefit, may be performed using techniques and procedures described herein and routinely practiced by a person skilled in the art. Exemplary methods include, but are not limited to, fluorescence cell-based assays of CFTR inhibition (see, e.g., Galietta et al., J. Physiol. 281:C 1734-C 1742 (2001)), short circuit apical chloride ion current measurements and patch-clamp analysis (see, e.g., Muanprasat et al., J. Gen. Physiol. 124:125-37 (2004); Ma et al., J. Clin. Invest. 110:1651-58 (2002); see also, e.g., Carmeliet, Verh. K. Acad. Geneeskd. Belg. 55:5-26 (1993); Hamill et al., Pflugers Arch. 391:85-100 (1981)). The divalent hydrazide-polymer conjugate compounds, including the divalent hydrazide-PEG conjugate compounds, may also be analyzed in animal models, for example, a closed intestinal loop model of cholera, suckling mouse model of cholera, and in vivo imaging of gastrointestinal transit (see, e.g., Takeda et al., Infect. Immun. 19:752-54 (1978); see also, e.g., Spira et al., Infect. Immun. 32:739-747 (1981)).

As described herein, divalent hydrazide-polymer conjugate compounds having a structure of either formula I or II and the divalent hydrazide-PEG conjugate compounds having a structure of formula I(a) or subformulae I(b), I(c)-I(j) or of formula II(a) or subformulae II(b), II(c), II(d), II(e), and II(f) and II((C)-(F)) described herein are capable of inhibiting CFTR activity (i.e., inhibiting, reducing, decreasing, blocking transport of chloride ion in the CFTR channel or pore in a statistically significant or biologically significant manner) in a cell and may be used for treating diseases, disorders, and conditions that result from or are related to aberrantly increased CFTR activity. Accordingly, methods of inhibiting ion transport by CFTR are provided herein that comprise contacting a cell (e.g., a gastrointestinal cell) that comprises CFTR in the outer membrane of the cell (i.e., a cell that expresses CFTR and has channels or pores formed by CFTR in the cell membrane) with any one or more of the conjugate compounds described herein, under conditions and for a time sufficient for CFTR and the conjugate compound to interact.

In certain embodiments, the cell is contacted in an in vitro assay, and the cell may be obtained from a subject or from a biological sample. A biological sample may be a blood sample (from which serum or plasma may be prepared and cells isolated), biopsy specimen, body fluids (e.g., lung lavage, ascites, mucosal washings, synovial fluid), bone marrow, lymph nodes, tissue explant, organ culture, or any other tissue or cell preparation from a subject or a biological source. A sample may further refer to a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. The subject or biological source may be a human or non-human animal, a primary cell culture (e.g., immune cells, virus infected cells), or culture adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, and the like.

As described herein the divalent hydrazide-polymer conjugate compounds, including the divalent hydrazide-PEG compounds, are CFTR inhibitors, and are useful in the treatment of a CFTR-mediated or associated condition, i.e., any condition, disorder or disease, that results from activity of CFTR, such as CFTR activity in ion transport. Such conditions, disorders, and diseases, are amenable to treatment by inhibition of CFTR activity, e.g., inhibition of CFTR ion transport.

In one embodiment, divalent hydrazide-polymer conjugate compounds having a structure of either formula I or II and the divalent hydrazide-PEG conjugate compounds having a structure of formula I(a) or subformulae I(b), I(c)-I(j) or of formula II(a) or subformulae II(b), II(c), II(d), II(e), and II(f) and II((C)-(F)) and specific structures described herein are used in the treatment of conditions associated with aberrantly increased intestinal secretion, particularly acute aberrantly increased intestinal secretion, including secretory diarrhea. Diarrhea amenable to treatment using divalent hydrazide conjugate compounds can result from exposure to a variety of pathogens or agents including, without limitation, cholera toxin (Vibrio cholerae), E. coli (particularly enterotoxigenic (ETEC)), Shigella, Salmonella, Campylobacter, Clostridium difficile, parasites (e.g., Giardia, Entamoeba histolytica, Cryptosporidiosis, Cyclospora), or diarrheal viruses (e.g., rotavirus). Secretory diarrhea resulting from an increased intestinal secretion mediated by CFTR may also be a disorder or sequelae associated with food poisoning, or exposure to a toxin including an enterotoxin such as cholera toxin, a E. coli toxin, a Salmonella toxin, a Campylobacter toxin, or a Shigella toxin.

Other secretory diarrheas that may be treated by administering any one or more of the divalent hydrazide PEG conjugates described herein include diarrhea associated with or that is a sequelae of AIDS, diarrhea that is a condition related to the effects of anti-AIDS medications such as protease inhibitors, diarrhea that is a condition or is related to administration of chemotherapeutic compounds, inflammatory gastrointestinal disorders, such as ulcerative colitis, inflammatory bowel disease (IBD), Crohn's disease, diverticulosis, and the like. Intestinal inflammation modulates the expression of three major mediators of intestinal salt transport and may contribute to diarrhea in ulcerative colitis both by increasing transepithelial Cl⁻ secretion and by inhibiting the epithelial NaCl absorption (see, e.g., Lohi et al., Am. J. Physiol. Gastrointest. Liver Physiol. 283:G567-75 (2002)).

Thus, one or more of the divalent hydrazide-polymer conjugate compounds having a structure of either formula I or II and the divalent hydrazide-PEG conjugate compounds having a structure of formula I(a) or subformulae I(b), I(c)-I(j) or of formula II(a) or subformulae II(b), II(c), II(d), II(e), and II(f) and II((C)-(F)) and specific structures described herein may be administered in an amount effective to inhibit CFTR ion transport and, thus, decrease intestinal fluid secretion. In such embodiments, at least one or more of the conjugate compounds are generally administered to a mucosal surface of the gastrointestinal tract (e.g., by an enteral route, e.g., oral, intraintestinal, rectal, and the like) or to a mucosal surface of the oral or nasal cavities, or (e.g., intranasal, buccal, sublingual, and the like).

Methods are provided herein for treating a disease or disorder associated with aberrantly increased ion transport by cystic fibrosis transmembrane conductance regulator (CFTR), wherein the methods comprise administering to a subject any one (or more) of the divalent hydrazide-polymer conjugate compounds having a structure of either formula I or II or the divalent hydrazide-PEG conjugate compounds having a structure of formula I(a) or subformulae I(b), I(c)-I(j) or of formula II(a) or subformulae II(b), II(c), II(d), II(e), and II(f) and II((C)-(F)), and specific structures described herein, wherein ion transport (particularly chloride ion transport) by CFTR is inhibited. A subject in need of such treatment includes humans and non-human animals. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

### Pharmaceutical Compositions

Also provided herein are pharmaceutical compositions comprising the divalent hydrazide-polymer conjugate compounds, including divalent hydrazide-PEG conjugate compounds, having a structure of any one of formulae I or I(a) or subformulae I(b), I(c)-I(j) or of formulae II or II(a) or subformulae II(b), II(c), II(d), II(e), and II(f) and II((C)-(F)), or specific structures described herein. The compound conjugates may be formulated in a pharmaceutical composition for use in treatment, which includes preventive treatment, of a disease or disorder manifested by increased intestinal fluid secretion, such as secretory diarrhea.

In pharmaceutical dosage forms, any one or more of the divalent hydrazide -polymer conjugate compounds (e.g., divalent hydrazide-PEG conjugate compounds, which include the divalent malonic hydrazide-PEG conjugate compounds and the divalent glycine hydrazide PEG conjugate compounds) described herein may be administered in the form of a pharmaceutically acceptable derivative, such as a salt, or they may also be used alone or in appropriate association, as well as in combination, with other pharmaceutically active compounds. The methods and excipients described herein are merely exemplary and are in no way limiting.

In one embodiment of particular interest, any one or more of the divalent hydrazide-polymer conjugate compounds (e.g., divalent hydrazide-PEG conjugate compounds, which include the divalent malonic hydrazide-PEG conjugate compounds and the divalent glycine hydrazide PEG conjugate compounds) may be delivered to the gastrointestinal tract of the subject to provide for decreased fluid secretion. Suitable formulations for this embodiment include any formulation that provides for delivery of the compound to the gastrointestinal surface, particularly an intestinal tract surface.

Optimal doses may generally be determined using experimental models and/or clinical trials. The optimal dose may depend upon the body mass, weight, or blood volume of the subject. In general, the amount of a conjugate compound described herein, such as a divalent hydrazide-PEG conjugate compound as described herein, that is included in a dose ranges from about 0.01 μg to about 1000 μg per kg weight of the host. The use of the minimum dose that is sufficient to provide effective therapy is usually preferred. Subjects may generally be monitored for therapeutic effectiveness using assays suitable for the condition being treated or prevented, which assays will be familiar to those having ordinary skill in the art and are described herein.

The dose of the composition for treating a disease or disorder associated with aberrant CFTR function, including but not limited to intestinal fluid secretion, secretory diarrhea, such as a toxin-induced diarrhea, or secretory diarrhea associated with or a sequelae of an enteropathogenic infection, Traveler's diarrhea, ulcerative colitis, irritable bowel syndrome (IBS), AIDS, chemotherapy and other diseases or conditions described herein may be determined according to parameters understood by a person skilled in the medical art. Accordingly, the appropriate dose may depend upon the subject's condition, that is, stage of the disease, general health status, as well as age, gender, and weight, and other factors considered by a person skilled in the medical art.

Pharmaceutical compositions may be administered in a manner appropriate to the disease or disorder to be treated as determined by persons skilled in the medical arts. An appropriate dose and a suitable duration and frequency of administration will be determined by such factors as the condition of the patient, the type and severity of the patient's disease, the particular form of the active ingredient, and the method of administration. In general, an appropriate dose (or effective dose) and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). Clinical assessment of the level of dehydration and/or electrolyte imbalance may be performed to determine the level of effectiveness of a conjugate compound and whether dose or other administration parameters (such as frequency of administration or route of administration) should be adjusted.

The terms, “treat” and “treatment” refer to both therapeutic treatment and prophylactic or preventative measures, wherein the objective is to prevent or slow or retard (lessen) an undesired physiological change or disorder or the expansion or severity of such disorder. As discussed herein, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival when compared to expected survival if a subject were not receiving treatment. Subjects in need of treatment include those already with the condition or disorder as well as subjects prone to have or at risk of developing the condition or disorder, and those in which the condition or disorder is to be prevented.

A pharmaceutical composition may be a sterile aqueous or non-aqueous solution, suspension or emulsion, which additionally comprises a physiologically acceptable excipient (pharmaceutically acceptable or suitable excipient or carrier) (i.e., a non-toxic material that does not interfere with the activity of the active ingredient). Such compositions may be in the form of a solid, liquid, or gas (aerosol). Alternatively, compositions described herein may be formulated as a lyophilizate, or compounds may be encapsulated within liposomes using technology known in the art. Pharmaceutical compositions may also contain other components, which may be biologically active or inactive. Such components include, but are not limited to, buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, stabilizers, dyes, flavoring agents, and suspending agents and/or preservatives.

Any suitable excipient or carrier known to those of ordinary skill in the art for use in pharmaceutical compositions may be employed in the compositions described herein. Excipients for therapeutic use are well known, and are described, for example, in Remington: The Science and Practice of Pharmacy (Gennaro, 21^{st} Ed. Mack Pub. Co., Easton, Pa. (2005)). In general, the type of excipient is selected based on the mode of administration. Pharmaceutical compositions may be formulated for any appropriate manner of administration, including, for example, topical, oral, nasal, intrathecal, rectal, vaginal, intraocular, subconjunctival, sublingual or parenteral administration, including subcutaneous, intravenous, intramuscular, intrasternal, intracavernous, intrameatal or intraurethral injection or infusion. For parenteral administration, the carrier preferably comprises water, saline, alcohol, a fat, a wax or a buffer. For oral administration, any of the above excipients or a solid excipient or carrier, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose and/or magnesium carbonate, may be employed.

A pharmaceutical composition (e.g., for oral administration or delivery by injection) may be in the form of a liquid. A liquid pharmaceutical composition may include, for example, one or more of the following: a sterile diluent such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition is preferably sterile.

A composition comprising any one of the divalent hydrazide-polymer conjugate compounds having a structure of either formula I or II and the divalent hydrazide-PEG conjugate compounds having a structure of formula I(a) or subformulae I(b), I(c)-I(j) or of formula II(a) or subformulae II(b), II(c), II(d), II(e), and II(f) and II((C)-(F)), and specific structures as described herein may be formulated for sustained or slow release. Such compositions may generally be prepared using well known technology and administered by, for example, oral, rectal or subcutaneous implantation, or by implantation at the desired target site. Sustained-release formulations may contain a conjugate compound dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of active conjugate compound contained within a sustained release formulation depends upon the site of implantation, the rate and expected duration of release, and the nature of the condition to be treated or prevented.

For oral formulations, the conjugate compounds described herein can be used alone or in combination with appropriate additives to make tablets, powders, granules or capsules, for example, with conventional additives, such as lactose, mannitol, corn starch or potato starch; with binders, such as starch, gelatin, natural sugars such as glucose or beta-lactose, corn sweeteners, natural and synthetic gums such as acacia, tragacanth, or sodium alginate, carboxymethylcellulose, polyethylene glycol, waxes, crystalline cellulose, cellulose derivatives, and acacia; with disintegrators, such as corn starch, potato starch or sodium carboxymethylcellulose, methyl cellulose, agar, bentonite, or xanthan gum; with lubricants, such as talc, sodium oleate, magnesium stearate sodium stearate, sodium benzoate, sodium acetate, or sodium chloride; and if desired, with diluents, buffering agents, moistening agents, preservatives, coloring agents, and flavoring agents. The conjugate compounds may be formulated with a buffering agent to provide for protection of the compound from low pH of the gastric environment and/or an enteric coating. The conjugate compounds may be formulated for oral delivery with a flavoring agent, e.g., in a liquid, solid or semi-solid formulation and/or with an enteric coating.

Oral formulations may be provided as gelatin capsules, which may contain the active compound conjugate along with powdered carriers, such as lactose, starch, cellulose derivatives, magnesium stearate, stearic acid, and the like. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract. Liquid dosage forms for oral administration may contain coloring and/or flavoring agents to increase acceptance of the compound by the subject.

The divalent hydrazide polymer conjugate compounds described herein can be made into suppositories by mixing with a variety of bases such as emulsifying bases or water-soluble bases. The conjugate compounds described herein can be administered rectally via a suppository. The suppository can include vehicles such as cocoa butter, carbowaxes and polyethylene glycols, which melt at body temperature, yet are solidified at room temperature.

The conjugate compounds described herein may be used in aerosol formulation to be administered via inhalation. The compounds may be formulated into pressurized acceptable propellants such as dichlorodifluoromethane, propane, nitrogen and the like.

Any one or more of the divalent hydrazide conjugate compounds described herein may be administered topically (e.g., by transdermal administration). Topical formulations may be in the form of a transdermal patch, ointment, paste, lotion, cream, gel, and the like. Topical formulations may include one or more of a penetrating agent, thickener, diluent, emulsifier, dispersing aid, or binder. When the conjugate compound is formulated for transdermal delivery, the compound may be formulated with or for use with a penetration enhancer. Penetration enhancers, which include chemical penetration enhancers and physical penetration enhancers, facilitate delivery of the compound through the skin, and may also be referred to as “permeation enhancers” interchangeably. Physical penetration enhancers include, for example, electrophoretic techniques such as iontophoresis, use of ultrasound (or “phonophoresis”), and the like. Chemical penetration enhancers are agents administered either prior to, with, or immediately following compound administration, which increase the permeability of the skin, particularly the stratum corneum, to provide for enhanced penetration of the drug through the skin. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995); Lenneruas et al., J. Pharm. Pharmacol. 2002; 54(4):499-508; Karande et al., Pharm. Res. 2002; 19(5):655-60; Vaddi et al., J. Pharm. Sci. 2002 July; 91(7):1639-51; Ventura et al., J. Drug Target 2001; 9(5):379-93; Shokri et al., Int. J. Pharm. 2001; 228(1-2):99-107; Suzuki et al., Biol. Pharm. Bull. 2001; 24(6):698-700; Alberti et al., J. Control Release 2001; 71(3):319-27; Goldstein et al., Urology 2001; 57(2):301-5; Kiijavainen et al., Eur. J. Pharm. Sci. 2000; 10(2):97-102; and Tenjarla et al., Int. J. Pharm. 1999; 192(2):147-58.

When a divalent conjugate compound is formulated with a chemical penetration enhancer, the penetration enhancer is selected for compatibility with the compound, and is present in an amount sufficient to facilitate delivery of the compound through skin of a subject, e.g., for delivery of the compound to the systemic circulation. The conjugate compounds may be provided in a drug delivery patch, e.g., a transmucosal or transdermal patch, and can be formulated with a penetration enhancer. The patch generally includes a backing layer, which is impermeable to the compound and other formulation components, a matrix in contact with one side of the backing layer, which matrix provides for sustained release, which may be controlled release, of the compound, and an adhesive layer, which is on the same side of the backing layer as the matrix. The matrix can be selected as is suitable for the route of administration, and can be, for example, a polymeric or hydrogel matrix.

For use in the methods described herein, one or more of the divalent hydrazide compounds described herein may be formulated with other pharmaceutically active agents or compounds, including other CFTR-inhibiting agents and compounds or agents and compounds that block intestinal chloride channels.

Kits with unit doses of the conjugate compounds described herein, usually in oral or injectable doses, are provided. In such kits, in addition to the containers containing the unit doses, will be an informational package insert describing the use and attendant benefits of the drugs in treating pathological condition of interest.

In another embodiment, a method of manufacture is provided for producing any one of the aforementioned divalent hydrazide-polymer conjugate compounds having a structure of either formula I or II and the divalent hydrazide-PEG conjugate compounds having a structure of formula I(a) or subformulae I(b), I(c)-I(j) or of formula II(a) or subformulae II(b), II(c), II(d), II(e), and II(f) and II((C)-(F)), and specific structures described herein. In one embodiment, the method of manufacture comprises synthesis of the compound. Synthesis of one of more of the compounds described herein may be performed according to methods described herein and practiced in the art. In another method of manufacture, the method comprises comprise formulating (i.e., combining, mixing) at least one of the compounds disclosed herein with a pharmaceutically suitable excipient. These methods are performed under conditions that permit formulation and/or maintenance of the desired state (i.e., liquid or solid, for example) of each of the compound and excipient. A method of manufacture may comprise one or more of the steps of synthesizing the at least one compound, formulating the compound with at least one pharmaceutically suitable excipient to form a pharmaceutical composition, and dispensing the formulated pharmaceutical composition in an appropriate vessel (i.e., a vessel appropriate for storage and/or distribution of the pharmaceutical composition).

Other embodiments and uses will be apparent to one skilled in the art in light of the present disclosures. The following examples are provided merely as illustrative of various embodiments and shall not be construed to limit the invention in any way.

### EXAMPLES

### Example 1

### Synthesis of MalH-PEG Conjugates

Synthesis of compound MalH-DIDS (2-naphthalenylamino-[(3,5-dibromo-2,4-dihydroxyphenyl)methylene]hydrazide [[[4-[2-(4-isothiocyanato-2-sulfopheacyl)ethenyl]-2-sulfophenyl]amino]thioxomethyl]hydrazide-propanedioic acid, disodium salt): A mixture of dihydrazide intermediate 4 (see above Reaction Scheme 1) (Sonawane et al., (2006), supra) (5 mmol) and 4,4′-diisothiocyanatostilbene-2,2′-disulfonic acid disodium salt hydrate (15 mmol) in DMF (5 ml) was refluxed for 4 h. After cooling, the reaction mixture was added dropwise to a stirred solution of EtOAc:EtOH (1:1), filtered, washed with ethanol, and further purified by column chromatography to give MalH-DIDS (43%) as a pale yellow solid.

¹H and ¹³C NMR spectra were obtained in CDCl₃ or DMSO-d₆ using a 400 MHz Varian Spectrometer referenced to CDCl₃ or DMSO. Mass spectrometry was performed using a WATERS LC/MS system (ALLIANCE HT 2790+ZQ, HPLC, WATERS model 2690, Milford, Mass.). Flash chromatography was performed using EM silica gel (230-400 mesh), and thin layer chromatography was performed on MERK silica gel 60 F254 plates (MERK, Darmstadt, Germany).

The MalH-DIDS compound had the following properties: mp >300° C.; ¹H NMR (DMSO-d₆): [symbol] 4.98, 5.63 (d, 1H, J=9.88, 8.51 Hz, COCH), 6.33-6.51 (m, 1H, Ar—H), 6.71, 6.84 (m, 1H, Ar—H), 7.03-7.37 (m, 4H, Ar—H & Ar—NH), 7.42-7.65 (m, 4H, Ar—H), 7.77-7.92 (m, 3H, Ar—H), 7.98-8.11 m, 1H), 8.93 (s, 1H), 9.13, 9.15, 9.21 (three s, 1H), 11.62, 11.70 (two s, 1H), 11.98, 12.00, 12.21 (s, 1H). All signals between 8.93-12.21 and 4.98, 5.63 were D₂O exchangable; MS (ES⁺) (m/z): [M−1]⁻ calculated for C₃₆H₂₅Br₂N₇O₉S₄, 987.71, found 986.44.

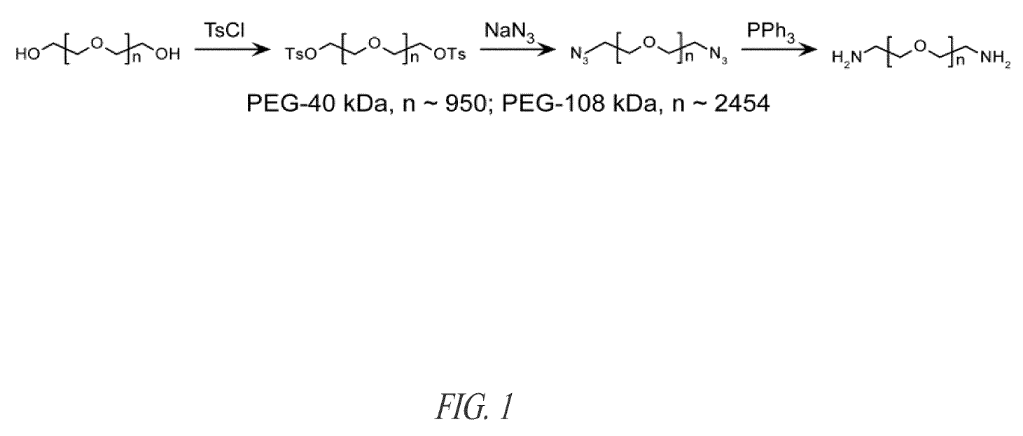

Both the divalent MalH-PEG-MalH and the monovalent MalH-PEG conjugates were synthesized by reaction of the corresponding bisamino and monoamino PEGs with a 5-fold molar excess of MalH-DIDS in anhydrous DMSO in presence of triethylamine as a base catalyst. Unreacted MalH-DIDS was removed by an amino-functionalized scavenger, and the PEG conjugates were purified by controlled precipitation and combinations of gel filtration, dialysis, ion exchange chromatography, and preparative HPLC. Bisamino-PEGs of up to 20 kDa were available commercially, giving solution lengths of up to 10 nm (see, e.g., Baird et al., Biochemistry. 42:12739-12748 (2003)), slightly less than that estimated for the distance between CFTR pores in a potential CFTR dimer. To generate larger conjugates with greater solutions lengths to potentially span inhibitor binding sites in CFTR dimers, available PEGs of 40 kDa and 108 kDa with terminal hydroxyls were converted to mesylates, followed by reaction with sodium azide and Staudinger reduction (see, e.g., Staudinger and Meyer, J. Helv. Chim. Acta. 2:635-646 (1919); and Pal et al., Synth. Commun. 34:1317-1323 (2004)), as shown in FIG. 1(C).

In greater detail, bis-amino or mono-amino PEGs (0.25, 1, 2, 3, 6, 10, 20 kDa, purchased from SIGMA-ALDRICH, St. Louis, Mo.; 40 and 100 kDa were synthesized as described below) (each 20 mg in 0.5 ml DMSO), MalH-DIDS (5 molar excess, Sonawane et al., 2007), and triethylamine (5-fold molar excess) were stirred slowly at room temperature for 1 hour. The amino-functionalized silica gel (10-fold molar excess) was added and stirred for additional 2 hours. The reaction mixture was filtered, scavenger was washed with 1 ml of DMSO, and the combined filtrate was added dropwise with stirring to 50 ml methanol. The precipitated product was filtered and washed twice with methanol. PEG conjugates of size 6 kDa and lower were further purified by anion exchange chromatography (Sepharose, GE) with NaCl gradient (0.5-1 M) elution. PEG conjugates of sizes 10 kDa, 20 kDa, 40 kDa, and 100 kDa were dialyzed overnight dialysis against PBS. These larger PEG conjugates were purified by gel filtration (SEPHADEX G25).

Bis-amino-PEGs (40 & 100 kDa): To a mixture of PEG (25 μmol, 40 and 108 kDa, Sigma) and triethylamine (14 μl, 100 μmol) in 2-5 ml CH₂Cl₂, methane sulfonyl chloride (52 mmol) was added dropwise at 0° C. and stirred for 6 hours at room temperature. The reaction mixture was washed with sodium bicarbonate (50 mM, 2 ml) and the organic phase was dried (MgSO₄). The evaporated organic phase yielded 1,w-dimethanesulfonylpolyoxyethylenes of 40 and 100 kDa, which were dissolved in 2 ml DMSO and NaN₃ (13 mg, 0.2 mmol) was added and stirred for 6 hours at 50° C. After cooling, water (20 ml) was added and the PEG-azide was extracted in dichloromethane and evaporated. A mixture of the PEG-azide (10 gmol) and triphenylphosphine (8 mg, 30 gmol) in dry methanol (3 mL) was refluxed for 1 hour and solvent was removed under reduced pressure. The residue was dissolved in dichloromethane (10 ml), filtered, and then exposed to dry hydrogen chloride gas. The precipitated hydrochloride salt of bis-amino PEG was filtered. The solution was cooled at 4° C. overnight and the precipitated hydrogen chloride salt was further purified by cation exchange chromatography with carboxymethyl CM-Sephadex C25, eluted with 10 mM Tris pH 9.0 and a 2 L gradient of 0.1-2 M NaCl.

Compound purity and absence of unreacted MalH-DIDS were confirmed by HPLC/MS, and the PEG conjugates were characterized by ¹H NMR, mass spectrometry, and UV/visible spectrometry. ¹H and ¹³C NMR spectra were obtained in CDCl₃ or DMSO-d₆ using a 400 MHz Varian Spectrometer referenced to CDCl₃ or DMSO. Mass spectrometry was done on a WATERS LC/MS system (ALLIANCE HT 2790+ZQ, HPLC: Waters model 2690, Milford, Mass.). Flash chromatography was performed using EM silica gel (230-400 mesh), and thin layer chromatography was done on MERK silica gel 60 F254 plates.

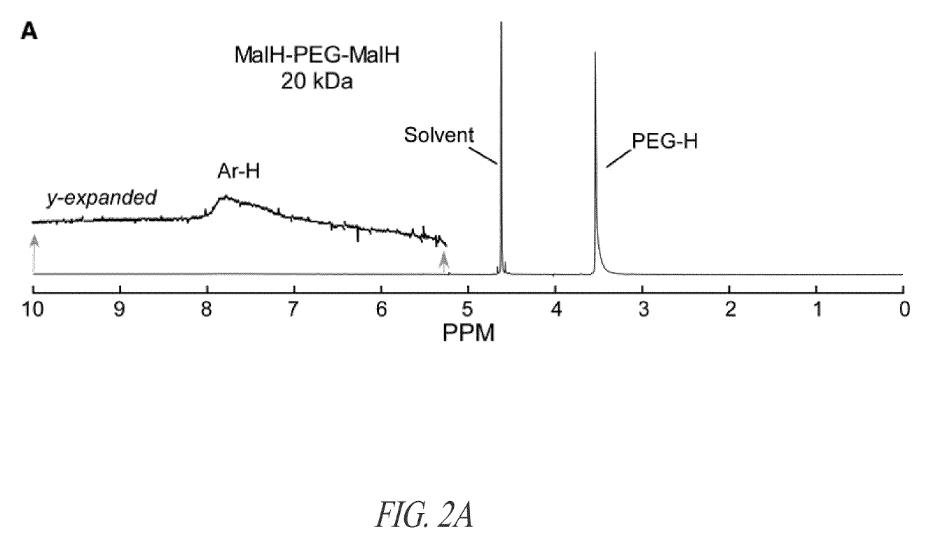

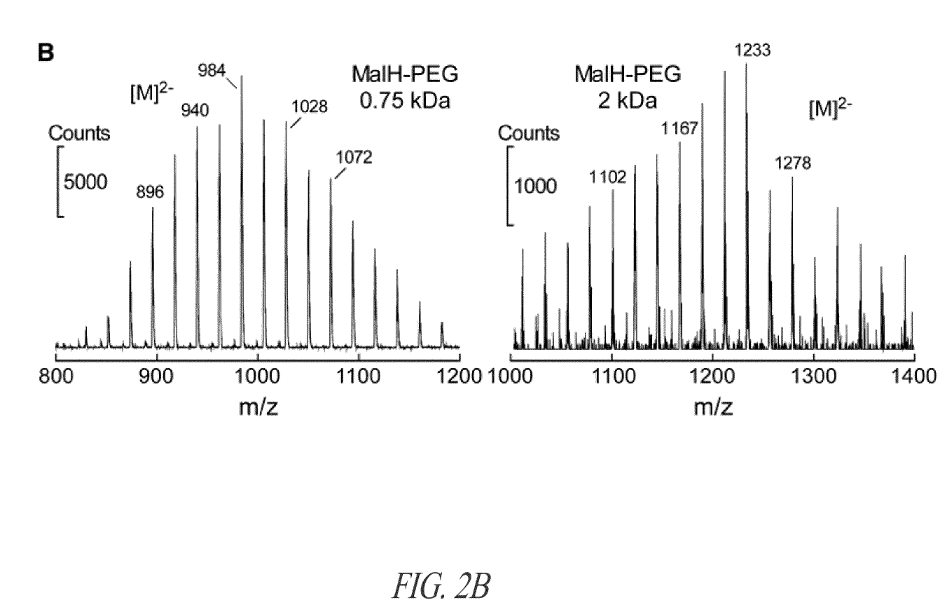

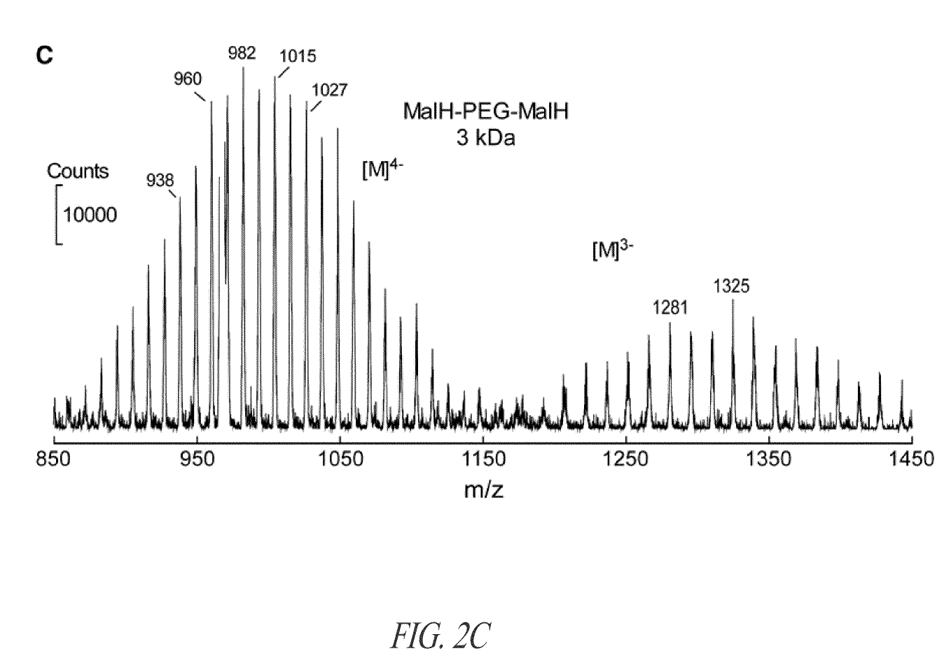

FIG. 2(A) shows a representative ¹H NMR spectrum of MalH-PEG20 kDa-MalH, showing a prominent peak for the PEG protons and relatively small peaks in the aromatic region seen after y-scale expansion. Similar NMR spectra were obtained for the other MalH-PEG conjugates. Mass spectra of the monovalent conjugates, MalH-PEG750-OMe (also called MalH-PEG, 0.75 kDa) and MalH-PEG2 kDa-OMe, and the divalent conjugate, MalH-PEG3 kDa-MalH, are provided in FIGS. 2(B) and 2(C). The monovalent conjugates are also called herein, MalH-PEG, followed by reference to the molecular weight of PEG of the particular conjugate (e.g., MalH-PEG, 0.75 kDa and MalH-PEG, 2 kDa). The divalent conjugates are also called herein MalH-PEG-MalH, followed by reference to the molecular weight of PEG of the particular conjugate. Mass spectra confirmed the predicted molecular weights. Higher molecular weight PEG conjugates had considerable polydispersity, with the expected characteristic peak spacing of CH₂—CH₂—O=44 Da/charge. The bisamino-PEGs were confirmed by ¹H NMR, giving multiple peaks for CH₂—NH₂ in the range 2.90-3.10 ppm, and ¹³C NMR showing C—NH₂ at ˜40 ppm (rather than ˜60 ppm for C—OH).

MalH-PEG0.1kDa-OH: yield 49%; mp >300° C.; ¹H NMR (D₂O): δ 3.19-4.44 (s, 8H, PEG-CH₂), 4.72, 5.22 (d, m, 1H, COCH), 7.60-7.88 (m, Ar—H), MS (ES⁻) (m/z): [M-2H]²⁻ and [M−1]⁻ calculated for C₄₀H₃₈Br₂N₈O₁₁S₄, 546.43 and 1094.86, found 545, 546, 547 [M−2H]²⁻, 1091, 1093, 1095 [M−H]⁻.

MalH-PEG0.75 kDa-OMe: yield 18%; ¹H NMR (D₂O): δ 2.85 (s, OCH₃), 3.52 (s, PEG-CH₂), 7.60-7.82 (m, Ar—H), MS (ES⁺) (m/z): [[M]²⁻+Na⁺]/2 calculated for C₆₉H₉₆Br₂N₈O₂₅S₄, 896.31, found 896+/−22, 44, 88, 176 (FIG. 2B).

MalH-PEG2 kDa-OMe: yield 31%; ¹H NMR (D₂O): δ 2.69 (s, O—CH₃), 3.21 (s, PEG-CH₂), 7.57-7.90 (m, Ar—H), MS (ES⁺) (m/z): [M−2H]²⁻ calculated for C₁₂₁H₂₀₀Br₂N₈O₅₁S₄, 1433.0, found 1432.8+/−22, 44, 88, 176 (FIG. 2B).

MalH-PEG5 kDa-OMe: yield 53%; ¹H NMR (D₂O): δ 2.64 (s, O—CH₃), 3.50 (s, PEG-CH₂), 7.38-7.91 (m, Ar—H); Conjugation ratio, MalH:PEG 1: 1.04 (UV/Visible).

MalH-PEG10kDa-OMe: yield 62%; ¹H NMR (D₂O): δ 2.58 (s, O—CH₃), 3.49 (s, PEG-CH₂), 7.55-8.07 (m, Ar—H); Conjugation ratio, MalH:PEG 1: 1.08 (UV/Visible).

MalH-PEG20kDa-OMe: yield 39%; ¹H NMR (D₂O): δ 2.59 (s, O—CH₃), 3.48 (s, PEG-CH₂), 7.40-7.96 (m, Ar—H); Conjugation ratio, MalH:PEG 1: 0.96 (UV/Visible).

MalH-PEG0.14 kDa-MalH: yield 29%; ¹H NMR (D₂O): δ 3.21-3.60 (m, PEG-CH₂), 3.62-3.71 (m, PEG-CH₂), 7.27-7.82 (m, Ar—H), MS (ES⁺) (m/z): [M−2H]²⁻ & [[M−2H]²⁻ 3Na⁺] calculated for C₇₈H₇₀Br₄N₁₆O₂OS₈, 1062.82 & 1131.82, found 1062.94 & 1130.88.

MalH-PEG3kDa-MalH: yield 44%; ¹H NMR (D₂O): δ 3.48 (s, PEG-CH₂), 7.13-780 (m, Ar—H), MS (ES⁺) (m/z): [[M−4H]⁴⁻+Na⁺]/4 calculated for C₂₂₄H₃₆₂Br₄N₁₆O₉₃S₈, 1339.25, found 1339 (FIG. 2B).

MalH-PEG6kDa-MalH: yield 26%; ¹H NMR (D₂O): δ 3.51 (s, PEG-CH₂), 7.22-8.14 (m, Ar—H); Conjugation ratio, MalH:PEG 2:1.11 (UV/Visible).

MalH-PEG10kDa-MalH: yield 23%; ¹H NMR (D₂O): δ 3.46 (s, PEG-CH₂), 7.05-8.21 (m, Ar—H); Conjugation ratio, MalH:PEG 2:0.92 (UV/Visible).

MalH-PEG20kDa-MalH: yield 55%; ¹H NMR (D₂O): δ 3.53 (s, PEG-CH₂), 7.14-7.91 (m, Ar—H); Conjugation ratio, MalH:PEG 2:1.07 (UV/Visible).

MalH-PEG40kDa-MalH: yield 27%; ¹H NMR (D₂O): δ 3.53 (s, PEG-CH₂), 7.13-8.12 (m, Ar—H); Conjugation ratio, MalH:PEG 2:0.95 (UV/Visible).

MalH-PEG108kDa-MalH: yield 58%; ¹H NMR (D₂O): δ 3.60 (s, PEG-CH₂), 7.07-7.89 (m, Ar—H); Conjugation ratio, MalH:PEG 2:1.08 (UV/Visible).

H₂N-PEG 40 kDa-NH₂: 26% yield, ¹H NMR (D₂O): δ 2.91 (m, —CH₂—N), 3.27 (t, O—CH₂—C—N), 3.52 (s, PEG-CH₂).

H₂N-PEG108 kDa-NH₂: 38% yield, ¹H NMR (D₂O): δ 2.90 (m, —CH₂—N), 3.31 (t, O—CH₂—C—N), 3.51 (s, PEG-CH₂).

### Example 2

### Improved CFTR Inhibition by Divalent MalH-PEG Conjugates

Fluorescence cell-based assay of CFTR inhibition. CFTR inhibition by the MalH-PEG conjugates was determined by a fluorescence cell-based assay utilizing doubly transfected cells expressing human wild-type CFTR and a yellow fluorescent protein (YFP) iodide sensor, as described (see, e.g., Galietta, et al., J. Physiol. 281:C 1734-C1742 (2001)). Fisher rat thyroid (FRT) cells stably expressing wild-type human CFTR and YFP-H148Q were cultured on 96-well black-wall plates as described (see, e.g., Ma, et al., J. Clin. Invest. 110:1651-1658 (2002)). Cells in 96-well plates were washed three times, and then CFTR was activated by incubation for 15 minutes with an activating cocktail containing 10 μM forskolin, 20 μM apigenin, and 100 μM IBMX. Test compounds were added 5 minutes before assay of iodide influx in which cells were exposed to a 100 mM inwardly directed iodide gradient. YFP fluorescence was recorded for 2 seconds before and 12 seconds after creation of the iodide gradient. Initial rates of iodide influx were computed from the time course of decreasing fluorescence after the iodide gradient.

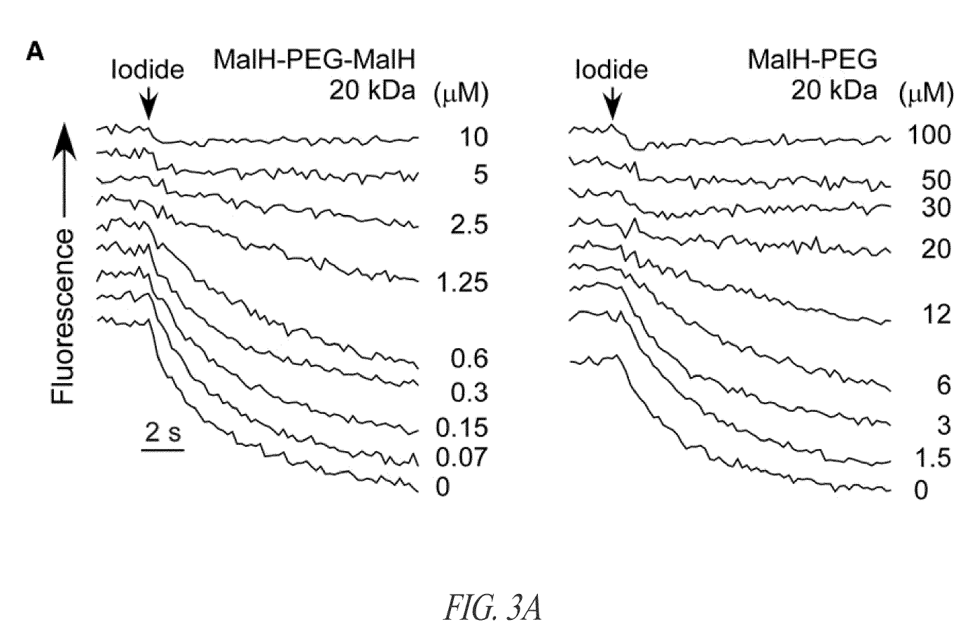

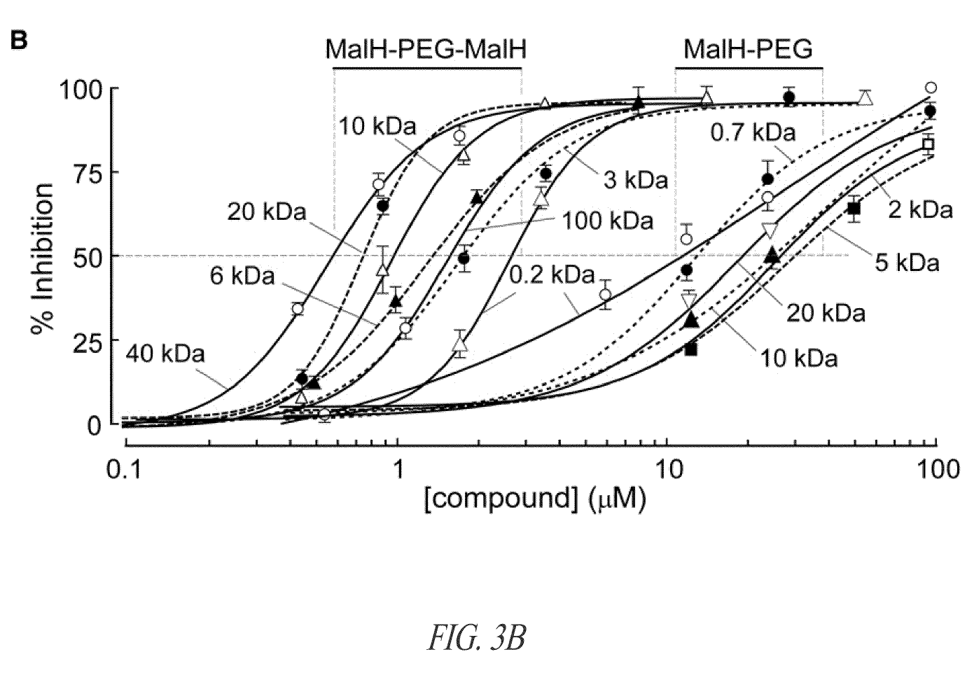

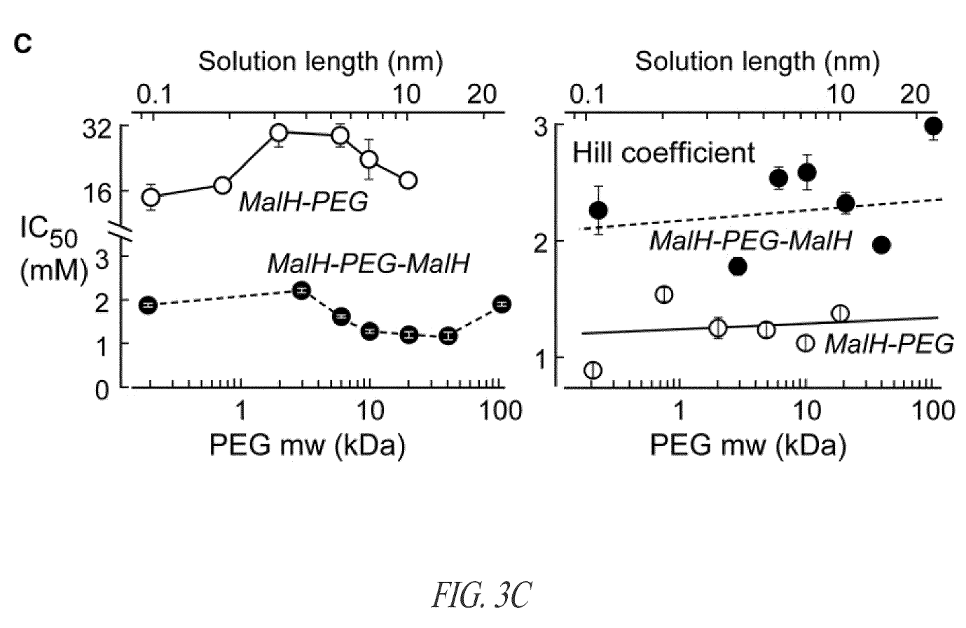

CFTR-facilitated iodide influx following extracellular iodide addition results in quenching of cytoplasmic YFP fluorescence. FIG. 3(A) shows representative original fluorescence data for conjugates of molecular size 20 kDa, showing substantially greater inhibition potency by the divalent (left panel) vs. monovalent (right panel) conjugate. FIG. 3(B) shows percentage CFTR inhibition as determined from initial curve slopes, for each of the monovalent and divalent MalH-PEG conjugates. FIG. 3(C) summarizes IC₅₀ values and Hill coefficients determined by non-linear regression to a single site inhibition model. Significantly lower IC₅₀ values were found for the divalent MalH-PEG-MalHs compared to the monovalent MalH-PEGs, with greater Hill coefficients, providing evidence for a cooperative mechanism for CFTR inhibition by the divalent conjugates, in which without wishing to be bound by theory, both MalH moieties in a divalent conjugate interact with CFTR.

Short-circuit current measurements. Short-circuit current measurements were performed to verify the apical membrane surface site of action and relatively potencies of the MalH-PEG conjugates, and to determine the kinetics of CFTR inhibition. FRT cells (stably expressing human wildtype CFTR) were cultured on Snapwell filters with 1 cm² surface area (Corning-Costar) to resistance >1,000 Ω·cm² as described (Sonowane et al., Gastroenterology, supra). Filters were mounted in an Easymount Chamber System (Physiologic Instruments, San Diego). For apical Cl⁻ current measurements the basolateral hemichamber contained 130 mM NaCl, 2.7 mM KCl, 1.5 mM KH₂PO₄, 1 mM CaCl₂, 0.5 mM MgCl₂, 10 mM Na-HEPES, 10 mM glucose (pH 7.3). The basolateral membrane was permeabilized with amphotericin B (250 μg/ml) for 30 min. In the apical solution 65 mM NaCl was replaced by sodium gluconate, and CaCl₂ was increased to 2 mM. Solutions were bubbled with 95% O₂/5% CO₂ and maintained at 37° C. Current was recorded using a DVC-1000 voltage-clamp (World Precision Instruments) using Ag/AgCl electrodes and 1 M KCl agar bridges.

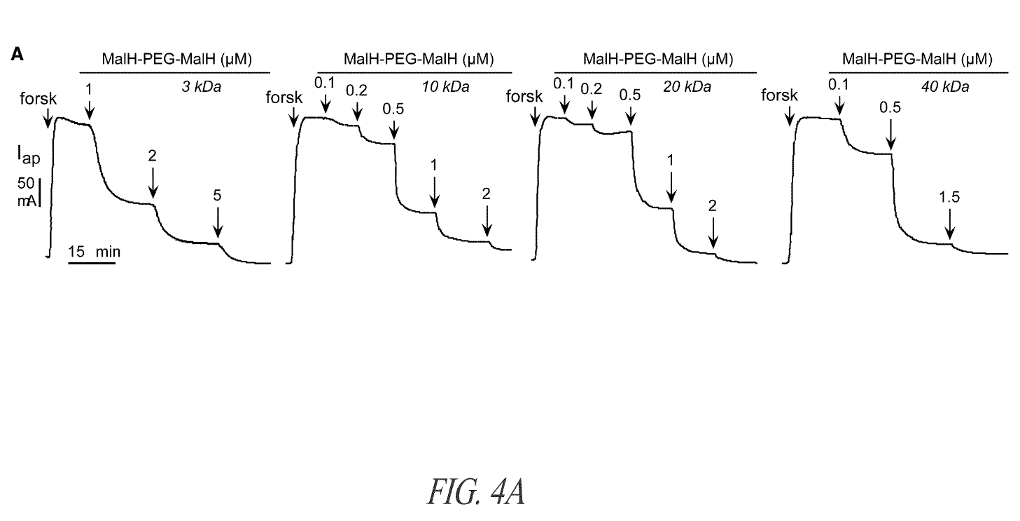

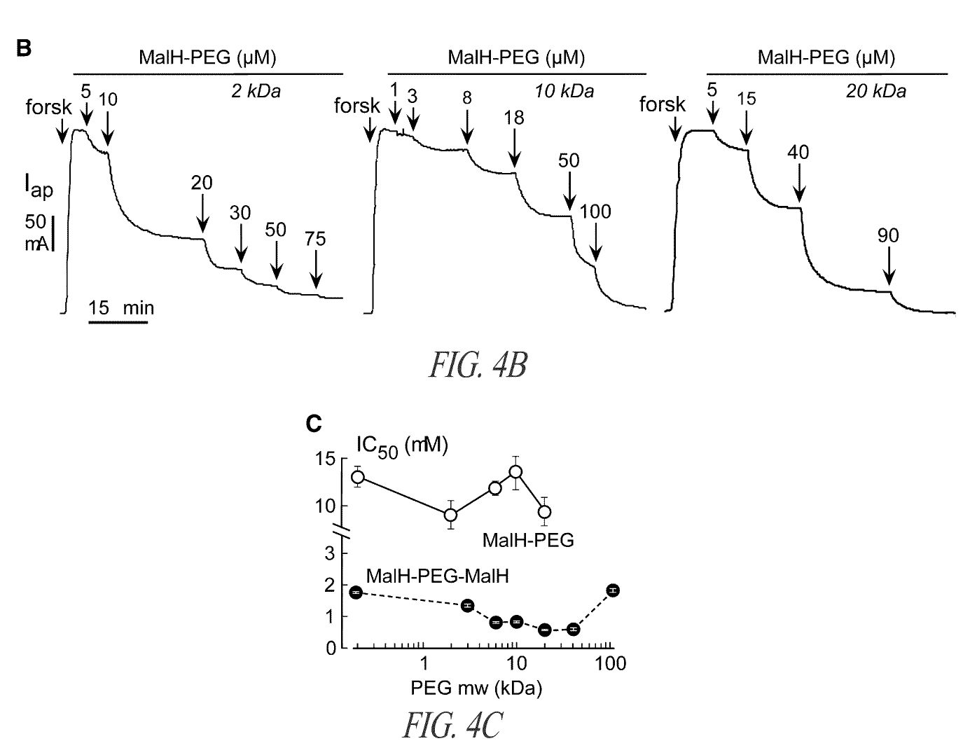

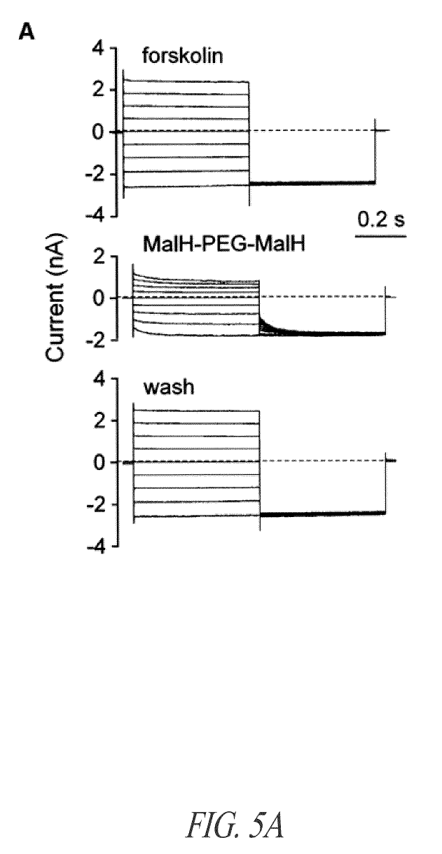

FIGS. 4(A) and 4(B) show representative short-circuit current data for inhibition of CFTR-mediated apical membrane chloride current by the divalent and monovalent MalH-PEGs, respectively. The conjugates were added only to the solution bathing the apical cell surface. Inhibition was rapid and was nearly complete at higher concentrations of the conjugates. CFTR chloride current was inhibited with IC₅₀ values of <1 μM for many of the divalent conjugates, whereas IC₅₀ values for the monovalent conjugates were generally >10 μM, as shown in FIG. 4(C), demonstrating a greater than 10-fold difference in IC₅₀ values between the monovalent and divalent conjugates. The exact IC₅₀ values obtained from each of the fluorescence assay and the short circuit current experiments differ because of differences in assay conditions, such as differences in apical membrane potential and dilution effects in the fluorescence assay.

### Example 3

### Mechanism of CFTR Inhibition by MalH-PEG Conjugates

Patch-clamp analysis. Whole-cell patch-clamp analysis was completed to investigate the mechanism of CFTR inhibition by the MalH-PEG conjugates. Experiments were performed to compare monovalent vs. divalent conjugates of molecular size 20 kDa, where IC₅₀ values differed by >20-fold. Whole-cell CFTR chloride currents were measured in the absence of inhibitors, and at concentrations near the IC₅₀ values of 0.6 μM and 15 μM for the divalent and monovalent conjugates, respectively.

Patch-clamp experiments were carried out at room temperature on FRT cells stably expressing wildtype CFTR. Whole-cell and outside-out configurations were used. For whole-cell experiments the pipette solution contained (in mM): 120 mM CsCl, 10 mM TEA-Cl, 0.5 mM EGTA, 1 mM MgCl₂, 40 mM mannitol, 10 mM Cs-HEPES and 3 mM mM MgATP (pH 7.3). For outside-out patches, the pipette solution contained (in mM): 150 mM N-methyl -D-glucamine chloride (NMDG-Cl), 2 mM MgCl₂, 10 mM EGTA, 10 mM Hepes, 1 mM ATP (pH 7.3). This pipette solution was supplemented with 125 nM catalytic subunit of protein kinase A. The bath solution in all experiments was (in mM): 150 NaCl, 1 CaCl₂, 1 MgCl₂, 10 glucose, 10 mannitol, 10 Na-Hepes (pH 7.4). The cell membrane was clamped at specified voltages using an EPC-7 patch-clamp amplifier (List Medical). Data were filtered at 500 Hz (whole cell) or 200 Hz (outside-out) and digitized at 1000 Hz using an INSTRUTECH ITC-16 AD/DA interface and the PULSE (HEKA) software. Inhibitors were applied by extracellular perfusion.

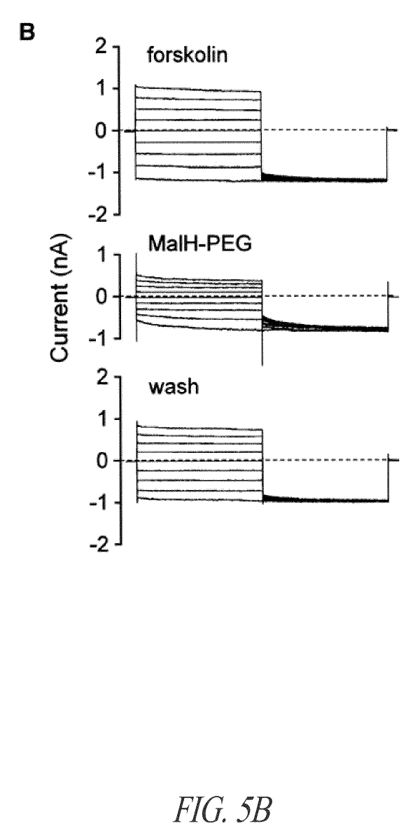

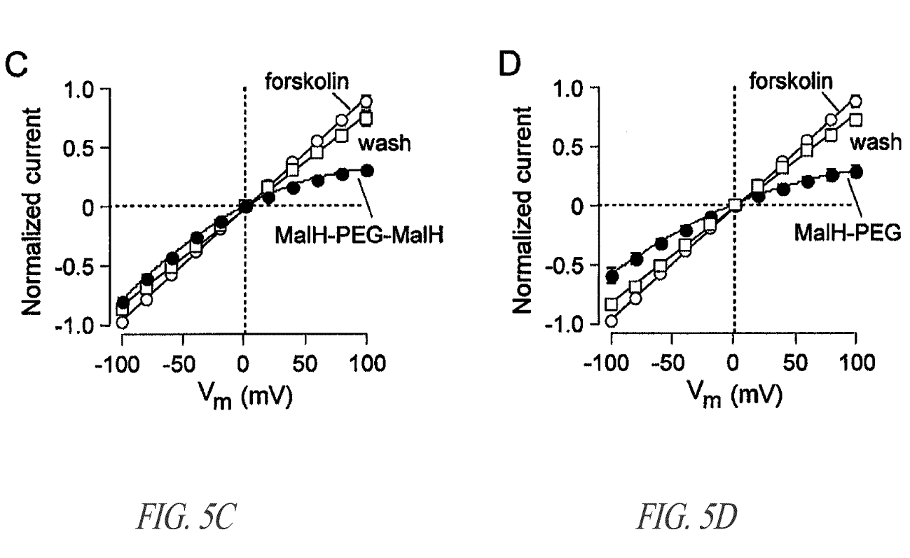

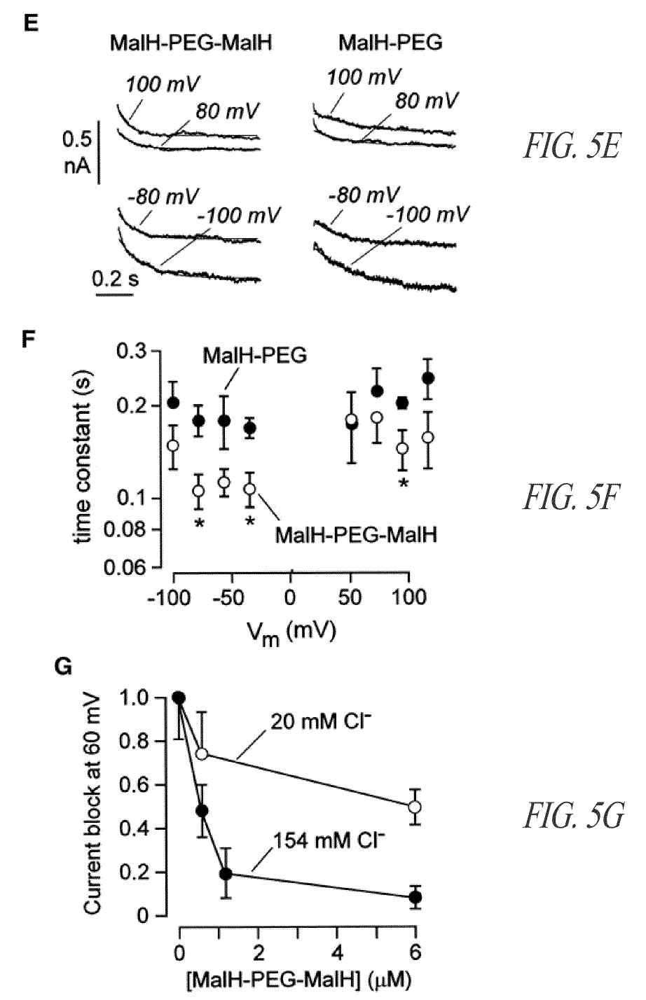

FIGS. 5(A) and 5(B) show representative traces, with averaged current-voltage relationships shown in FIGS. 5(C) and 5(D). Both compounds produced voltage-dependent inhibition of CFTR currents with positive currents being more strongly affected, producing inwardly rectifying behavior in the presence of inhibitors (which is consistent with occlusion of the channel pore). The divalent conjugate showed a more marked voltage-dependence. CFTR inhibition by the MalH-PEG conjugates was reversible following inhibitor washout with recovery to baseline current in 2-4 minutes.

The CFTR current traces at different membrane potentials revealed slow kinetics for block and unblock by the MalH-PEG conjugates. When membrane voltage was clamped from a holding potential of 0 mV to a positive or a negative potential, CFTR currents showed time-dependent decreases and increases, respectively, as shown in FIG. 5(E). The kinetics fitted well to single-exponential functions with time constants in the range 100-200 ms, substantially greater than that for GlyH-101 (8-10 ms; see, e.g., Muanprasat et al., J. Gen. Physiol. 124:125-137 (2004)), though comparable to those of MalH-lectin conjugates (see, e.g., Sonawane et al., Gastroenterology 132:1234-1244 (2007)). The time constants showed little voltage-dependence, and at most potentials were significantly greater for the monovalent compared with divalent conjugates, as shown in FIG. 5(F). As further evidence that the MalH-PEG conjugates act by a pore occlusion mechanism, lowering extracellular Cl⁻ to 20 mM, strongly reduced the block by MalH-PEG-MalH (see FIG. 5(G)).

The distance of the MalH binding site along the electric field was estimated with the Woodhull equation (see Woodhull, J. Gen. Physiol. 61:687-708 (1973)). Assuming a valence (z) value of −1 for both monovalent and divalent compounds, the computed fraction of the membrane potential sensed at the binding site relative to the extracellular surface (δ) is 0.21 and 0.33, respectively. If z is −2 for the divalent compounds, δ becomes 0.17.

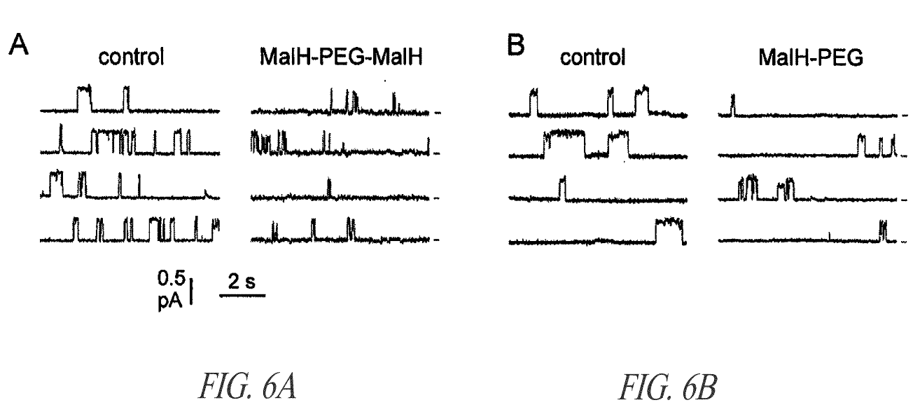

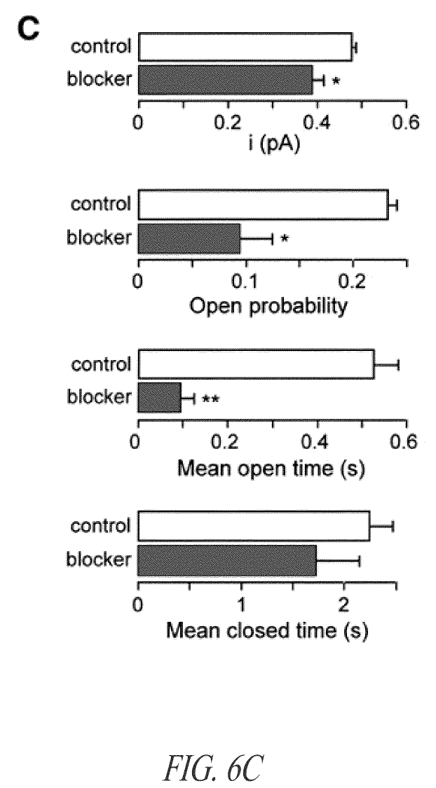

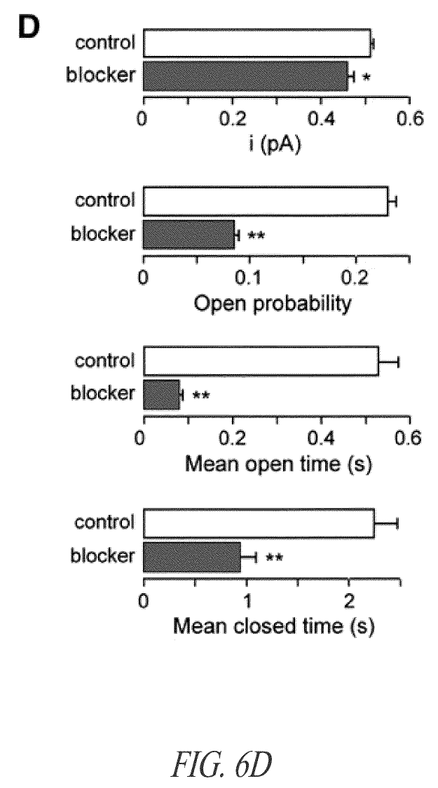

Outside-out patch-clamp measurements were carried out to further investigate the mechanism of CFTR inhibition by the MalH-PEG conjugates. To activate CFTR , the pipette (intracellular) solution contained 1 mM ATP and 5 μg/ml protein kinase A catalytic subunit. FIGS. 6(A) and 6(B) show representative recordings of single channel CFTR channel activity obtained at 60 mV in the absence and presence of divalent and monovalent MalH-PEG conjugates. Addition of MalH-PEG conjugates to the extracellular side greatly reduced the duration of channel openings. Data from multiple experiments are summarized in FIGS. 6(C) and 6(D). The MalH-PEG conjugates significantly reduced mean open time and apparent open channel probability. The mean closed time was significantly reduced by the monovalent MalH-PEG, without wishing to be bound by theory, probably because of an increased number of brief intraburst closures although a more detailed analysis requires different experimental parameters . A significant decrease (˜10%) in single channel amplitude (I) was also observed. These results support the conclusion that MalH-PEG conjugates inhibit CFTR by an external pore occlusion mechanism.

### Example 4

### Divalent MalH-PEG Conjugates Inhibit Cholera Toxin-Induced Intestinal Fluid Secretion

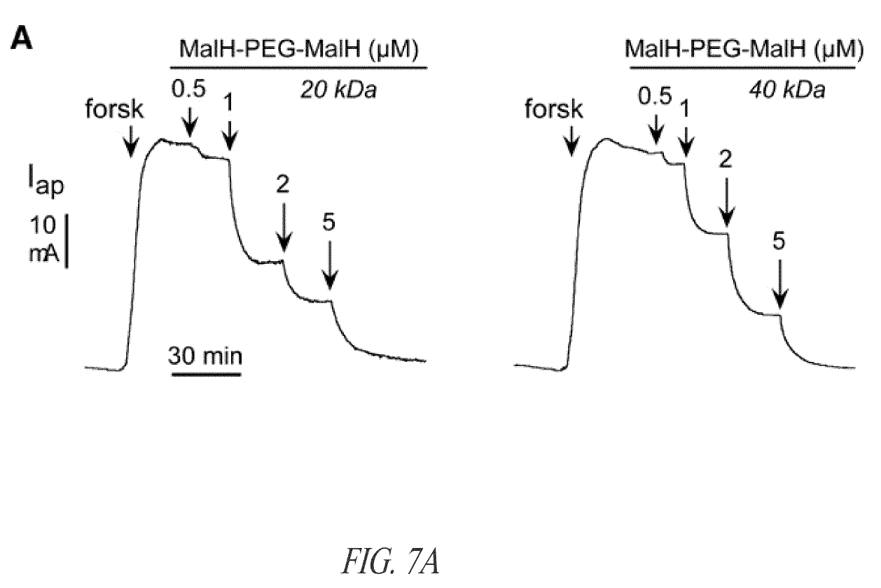

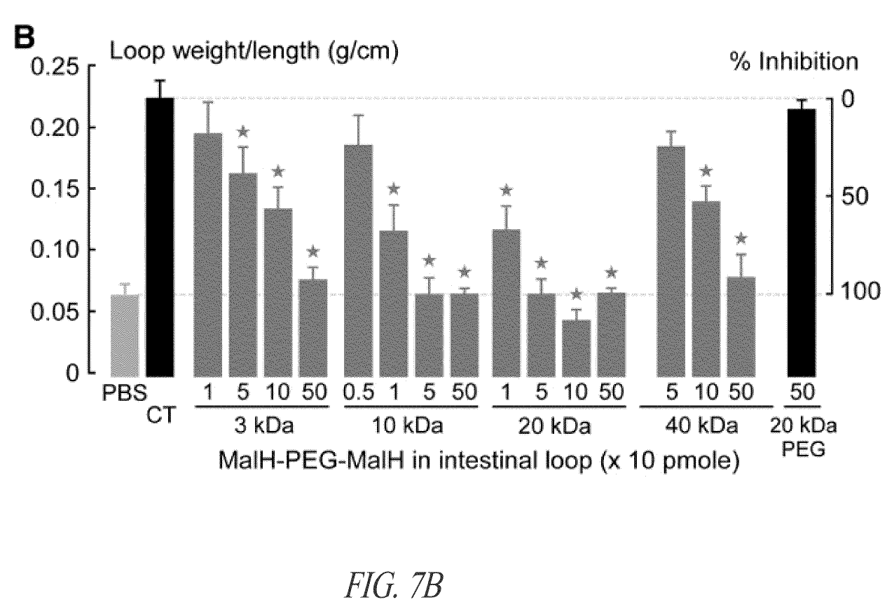

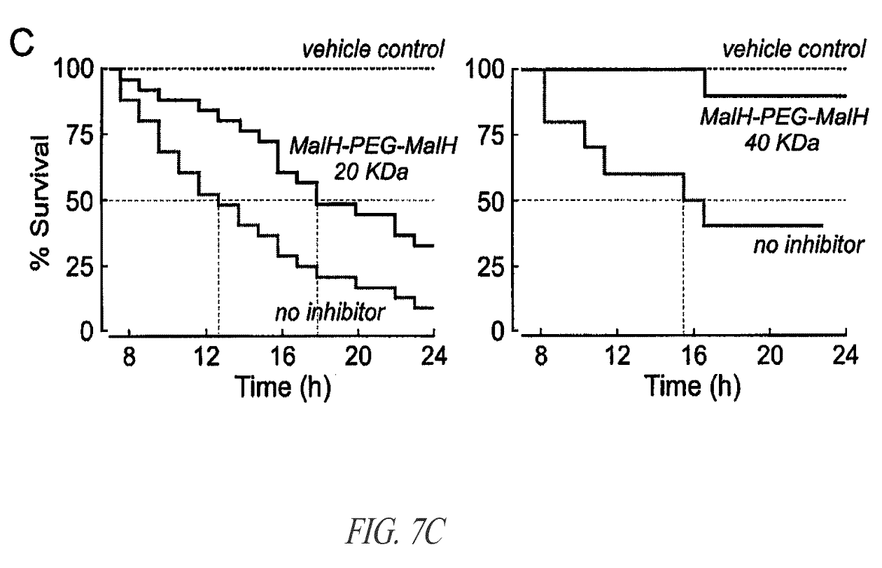

In vitro cell model of fluid secretion. Inhibition efficacy of divalent conjugates was investigated in T84 colonic epithelial cells under non-permeabilized conditions and in the absence of a Cl⁻ gradient. Following epithelial sodium channel (EnaC) inhibition by amiloride, CFTR was activated by forskolin, and then MalH-PEG-MalHs were added to the chamber bathing the apical cell surface. FIG. 7(A) shows inhibition of forskolin-stimulated short circuit current by 20 kDa MalH-PEG20 kDa-MalH (left) and 40 kDa MalH-PEG20 kDa-MalH (right) in T84 cells with IC₅₀ values of ˜1 μM.

Closed intestinal loop model of cholera. The divalent MalH-PEG conjugates were tested for their antisecretory efficacy in an intestinal closed-loop mouse model of cholera. The closed-loop model quantifies the accumulation of fluid in mid-jejunal loops in response to cholera toxin. This is a well-established and technically simple quantitative model in which fluid secretion and absorption mechanisms are intact, though there is no intestinal transit (see, e.g., Oi et al., Proc. Natl. Acad. Sci. USA. 99:3042-3046 (2002)).

Midjejunal loops were injected either with saline or with cholera toxin containing different concentrations of test compound, and intestinal fluid secretion was measured at 6 hours. Mice (CD1 strain, 28-34g) were given 5% sucrose for 24 h prior to anaesthesia (2.5% avertin intraperitoneally). Body temperature was maintained at 36-38° C. using a heating pad. Following a small abdominal incision three or four closed mid-jejunal loops (length 15-20 mm) were isolated by sutures. Loops were injected with 100 μl of PBS or PBS containing cholera toxin (1 μg), without or with test compounds. The abdominal incision was closed with suture and the mice were allowed to recover from anesthesia. At 6 hours the mice were again anesthetized, the intestinal loops were removed, and loop length and weight were measured to quantify net fluid accumulation. Mice were sacrificed by an overdose of avertin. All protocols were approved by the UCSF Committee on Animal Research.

FIG. 7(B) shows a loop weight-to-length ratio of 0.06 g/cm in PBS-injected loops (corresponding to 100% inhibition), and ˜0.22 g/cm for cholera toxin-injected loop (corresponding to 0% inhibition). The divalent MalH-CFTR conjugates of molecular sizes 2, 10, 20 and 40 kDa inhibited cholera toxin-induced fluid secretion in a concentration-dependent manner with IC₅₀ values of ˜100, 10, 10 and 100 pmol/loop, respectively. PEG alone (bar at right) did not inhibit intestinal fluid accumulation.

Suckling mouse model of cholera. The divalent MalH-PEG conjugates were also tested for their antisecretory efficacy in an art-accepted suckling mouse model of cholera, in which survival is the endpoint for intestinal fluid loss (see, e.g., Sonawane et al., FASEB J. 20:130-132 (2006); and Takeda et al., Infect. Immun. 19:752-754 (1978)). Equal numbers of newborn Balb-C mice from the same mother(s), each weighing 2-3g (age 3-4 days), were gavaged using PE-10 tubing with 10 μg cholera toxin in a 50 μL volume containing 50 mM Tris, 200 mM NaCl and 0.08% Evans blue (pH 7.5), with or without MalH-PEG20 kDa-MalH (500 μmol) or MalH-PEG40 kDa-MalH (500 μmol). ‘Control’ mice were gavaged with buffer alone. Successful gavage was confirmed by Evans blue localization in stomach/intestine. Mouse survival was assessed hourly, as described (see, e.g., Sonawane et al., Gastroenterology 132:1234-1244 (2007)).

FIG. 7(C) summarizes the suckling mouse survival studies. Suckling, 3-4 day old Balb-C mice receiving a single oral dose of cholera toxin generally died by 20 hours, with no mortality in ‘vehicle control’ (saline gavaged) mice over >24 h. Survival of mice receiving cholera toxin was significantly improved when the divalent 20 or 40 kDa MalH-PEG-MalH conjugates were gavaged along with cholera toxin.

### Example 5

### CFTR Inhibitory Activity of Monovalent Hydrazide Compounds

The CFTR inhibitory activity of exemplary monomeric hydrazide compounds was determined as described (see U.S. Patent Application Publication No. 2005/023974). Presented in the following table, are exemplary glycine hydrazide compounds that exhibited CFTR inhibitory activity between 1 and 20 μM Kᵢ (the concentration that resulted in 50% inhibition of CFTR C₁₋conductance) as determined by short-circuit current analysis on CFTR-expressing FRT cells. CFTR inhibitory activity of exemplary malonic hydrazide compounds was between 1 and 10 μM Kᵢ and was determined by short-circuit current analysis on CFTR-expressing FRT cells (see U.S. Pat. No. 7,414,037; U.S. Patent Application Publication No. 2005/023974).

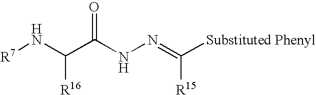

| Compound | R⁷ | R¹⁶ | Substituted Phenyl | R¹⁵ |
|---|---|---|---|---|
| GlyH-101 | 2-naphthalenyl | H | 3,5-di-Br-2,4-di-OH-Ph | H |
| GlyH-102 | 2-naphthalenyl | H | 3,5-di-Br-4-OH-Ph | H |
| GlyH-103 | 2-naphthalenyl | H | 3,5-di-Br-2-OH-4-OMe-Ph | H |
| GlyH-104 | 1-naphthalenyl | H | 3,5-di-Br-2,4-di-OH-Ph | H |
| GlyH-105 | 1-naphthalenyl | H | 3,5-di-Br-4-OH-Ph | H |
| GlyH-106 | 2-naphthalenyl | CH₃ | 3,5-di-Br-2,4-di-OH-Ph | H |
| GlyH-107 | 2-naphthalenyl | CH₃ | 3,5-di-Br-4-OH-Ph | H |
| GlyH-108 | 2-naphthalenyl | H | 3,5-di-Br-2,4-di-OH-Ph | CH₃ |
| GlyH-109 | 2-naphthalenyl | H | 3,5-di-Br-4-OH-Ph | CH₃ |
| OxaH-110 | 2-naphthalenyl | ═O | 3,5-di-Br-2,4-di-OH-Ph | H |
| OxaH-111 | 2-naphthalenyl | ═O | 3,5-di-Br-4-OH-Ph | H |
| OxaH-112 | 2-naphthalenyl | ═O | 3,5-di-Br-2,4-di-OH Ph | CH₃ |
| OxaH-113 | 2-naphthalenyl | ═O | 3,5-di-Br-4-OH-Ph | CH₃ |
| GlyH-114 | 4-Cl-Ph | H | 3,5-di-Br-4-OH-Ph | H |
| GlyH-115 | 4-Cl-Ph | H | 3,5-di-Br-2,4-di-OH Ph | H |
| GlyH-116 | 4-Me-Ph | H | 3,5-di-Br-2,4-di-OH Ph | H |

All the above U.S. patents, U.S. patent application publications, U.S. patent applications, foreign patents, foreign patent applications, and non-patent publications referred to in this specification and/or listed in the Application Data Sheet, are incorporated herein by reference, in their entirety.

From the foregoing a person skilled in the art will appreciate that, although specific embodiments have been described herein for purposes of illustration, various modifications may be made. Those skilled in the art will recognize, or be able to ascertain, using no more than routine experimentation, many equivalents to the specific embodiments described herein. Such equivalents are intended to be encompassed by the following claims. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having the following structure I: 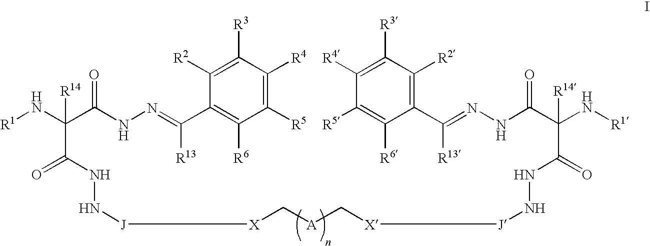
or a pharmaceutically acceptable salt, prodrug, or stereoisomer thereof
wherein:
R¹ and R^{1′} are the same or different and independently optionally substituted phenyl, optionally substituted heteroaryl, optionally substituted quinolinyl, optionally substituted anthracenyl, or optionally substituted naphthalenyl;
R², R^{2′}, R³, R^{3′}, R⁴, R^{4′}, R⁵, R^{5′}, R⁶, and R^{6′} are each the same or different and independently hydrogen, hydroxy, C₁₋₈ alkyl, C₁₋₈ alkoxy, carboxy, halo, nitro, cyano, —SO₃H, —S(═O)₂NH₂, aryl, and heteroaryl;
R¹³, R^{13′}, R¹⁴, and R^{14′} are each the same or different and independently hydrogen or C₁₋₈ alkyl;
X and X′ are each the same or different linker moiety;
J and J′ are each the same or different spacer moiety;
A is a polymer subunit; and
n is an integer between 0 and 2,500.

2. The compound of claim 1 wherein A is —CH₂—O—CH₂— and the compound has the following structure I(a): 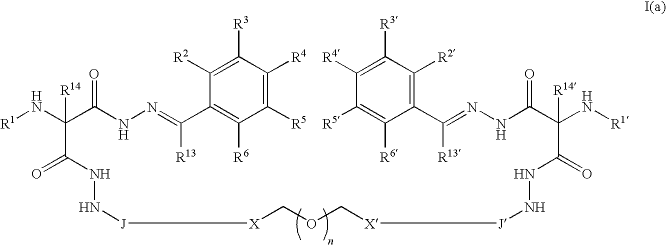
or a pharmaceutically acceptable salt, prodrug, or stereoisomer thereof
wherein:
R¹ and R^{1′} are the same or different and independently optionally substituted phenyl, optionally substituted heteroaryl, optionally substituted quinolinyl, optionally substituted anthracenyl, or optionally substituted naphthalenyl;
R², R^{2′}, R³, R^{3′}, R⁴, R^{4′}, R⁵, R^{5′}, R⁶, and R^{6′} are the same or different and independently hydrogen, hydroxy, C₁₋₈ alkyl, C₁₋₈ alkoxy, carboxy, halo, nitro, cyano, —SO₃H, —S(═O)₂NH₂, aryl, and heteroaryl;
R¹³, R^{13′}, R¹⁴, and R^{14′} are the same or different and independently hydrogen or C₁₋₈ alkyl;
X and X′ are each the same or different linker moiety;
J and J′ are each the same or different spacer moiety; and
n is an integer between 0 and 2,500.

3. The compound of claim 2 wherein R¹³, R^{13′}, R¹⁴, and R^{14′} are the same or different and independently hydrogen or methyl.

4. The compound of claim 2 wherein R¹ and R^{1′} are the same or different and independently phenyl substituted with one or more of hydroxy, C₁₋₈ alkyl, C₁₋₈ alkoxy, carboxy, —SO₃H, aryl, aryloxy, and halo.

5. The compound of claim 2 wherein R¹ and R^{1′} are the same or different and independently 1-naphthalenyl or 2-naphthalenyl, optionally substituted with one or more of halo, hydroxy, —SH, —SO₃H, C₁₋₈ alkyl, and C₁₋₈ alkoxy; aryloxy; mono-halophenyl; di-halophenyl; mono-alkylphenyl; 2-anthracenyl; or 6-quinolinyl.

6. The compound of claim 5 wherein R¹ and R^{1′} are the same or different and independently mono-(halo)naphthalenyl; di-(halo)naphthalenyl; tri-(halo)naphthalenyl; mono-(hydroxy)naphthalenyl; di-(hydroxy)naphthalenyl; tri-(hydroxy)naphthalenyl; mono-(alkoxy)naphthalenyl; di-(alkoxy)naphthalenyl; tri-(alkoxy)naphthalenyl; mono-(aryloxy)naphthalenyl; di-(aryloxy)naphthalenyl; mono-(alkyl)naphthalenyl; di-(alkyl)naphthalenyl; tri-(alkyl)naphthalenyl; mono-(hydroxy)-naphthalene-sulfonic acid; mono-(hydroxy)-naphthalene-disulfonic acid; mono(halo)-mono (hydroxy)naphthalenyl; di(halo)-mono(hydroxy)naphthalenyl; mono(halo)-di(hydroxy)naphthalenyl; di(halo)-di(hydroxy)naphthalenyl; mono-(alkyl)-mono-(alkoxy)-naphthalenyl; or mono-(alkyl)-di-(alkoxy)-naphthalenyl.

7. The compound of claim 4 wherein R¹ and R^{1′} are the same or different and independently mono-(halo)phenyl; di-(halo) phenyl; tri-(halo) phenyl; 2-halophenyl; 4-halophenyl; 2-4-halophenyl; mono-(hydroxy)phenyl; di-(hydroxy)phenyl tri-(hydroxy)phenyl; mono-(alkoxy)phenyl; di-(alkoxy)phenyl; tri-(alkoxy)phenyl; mono-(aryloxy)phenyl; di-(aryloxy)phenyl; mono-(alkyl)phenyl; di-(alkyl)phenyl; tri-(alkyl)phenyl; mono-(hydroxy)-phenyl-sulfonic acid; mono-(hydroxy)-phenyl-disulfonic acid; mono(halo)-mono(hydroxy)phenyl; di(halo)-mono(hydroxy)phenyl; mono(halo)-di(hydroxy)phenyl; di(halo)-di(hydroxy)phenyl; mono-(alkyl)-mono-(alkoxy)-phenyl; or mono-(alkyl)-di-(alkoxy)-phenyl.

8. The compound of claim 5 wherein R¹ and R^{1′} are the same or different and independently 2-naphthalenyl, 2-chlorophenyl; 4-chlorophenyl; 2-4-dichlorophenyl, 4-methylphenyl, 2-anthracenyl, or 6-quinolinyl.

9. The compound of claim 2 wherein R², R^{2′}, R³, R^{3′}, R⁴, R^{4′}, R⁵, R^{5′}, R⁶, and R^{6′} are the same or different and independently hydrogen, hydroxy, halo, C₁₋₈ alkyl, C₁₋₈ alkoxy, or carboxy.

10. The compound of claim 9 wherein R², R³, R⁴, R⁵, and R⁶, are each the same or different and independently selected such that the phenyl group to which R², R³, R⁴, R⁵, and R⁶ are attached is substituted with one, two, or three halo; one or two carboxy; one, two, or three hydroxy; one or two halo and one, two, or three hydroxy; one or two halo, one or two hydroxy, and one C₁₋₈ alkoxy; one or two halo, one hydroxy, and one or two C₁₋₈ alkoxy; or one halo, one or two hydroxy, and one or two C₁₋₈ alkoxy.

11. The compound of claim 9 wherein R^{2′}, R^{3′}, R^{4′}, R^{5′}, and R^{6′} are each the same or different and independently selected such that the phenyl group to which R^{2′}, R^{3′}, R^{4′}, R^{5′}, and R^{6′} are attached is substituted with one, two, or three halo; one or two carboxy; one, two, or three hydroxy; one or two halo and one, two, or three hydroxy; one or two halo, one or two hydroxy, and one C₁₋₈ alkoxy; one or two halo, one hydroxy, and one or two C₁₋₈ alkoxy; or one halo, one or two hydroxy, and one or two C₁₋₈ alkoxy.

12. The compound of claim 10 wherein halo is bromo.

13.-14. (canceled)

15. The compound of claim 10 wherein R², R³, R⁴, R⁵, and R⁶ are the same or different and independently selected such that the phenyl group to which R², R³, R⁴, R⁵, and R⁶ is attached is 2-, 3-, or 4-halophenyl; 3,5-dihalophenyl; 2-, 3-, or 4-hydroxyphenyl; 2,4-dihydroxyphenyl; 3,5-dihalo-2,4,6-trihydroxyphenyl, 3,5-dihalo-2,4-dihydroxyphenyl; 3,5-dihalo-4-hydroxyphenyl; 3-halo-4-hydroxyphenyl; 3,5-dihalo-2-hydroxy-4-methoxyphenyl; or 4-carboxyphenyl.

16. The compound of claim 11 wherein R^{2′}, R^{3′}, R^{4′}, R^{5′}, and R^{6′} are the same or different and independently selected such that the phenyl group to which R^{2′}, R^{3′}, R^{4′}, R^{5′}, and R^{6′} is attached is 2-, 3-, or 4-halophenyl; 3,5-dihalophenyl; 2-, 3-, or 4-hydroxyphenyl; 2,4-dihydroxyphenyl; 3,5-dihalo-2,4,6-trihydroxyphenyl, 3,5-dihalo-2,4-dihydroxyphenyl; 3,5-dihalo-4-hydroxyphenyl; 3-halo-4-hydroxyphenyl; 3,5-dihalo-2-hydroxy-4-methoxyphenyl; or 4-carboxyphenyl.

17. The compound of claim 11 wherein halo is bromo.

18. The compound of claim 9 wherein
(a) each of R³ and R⁵ is halo and each of R⁴ and R⁶ is hydroxy;
(b) each of R³ and R⁵ is halo and R⁴ is hydroxyl;
(c) each of R³ and R⁵ is bromo and each of R⁴ and R⁶ is hydroxyl; or
(d) each of R³ and R⁵ is bromo, R⁴ is hydroxy, and R⁶ is hydrogen.

19. The compound of claim 9 wherein
(a) each of R^{3′} and R^{5′} is halo and each of R^{4′} and R^{6′} is hydroxyl;
(b) each of R^{3′} and R^{5′} is halo and R^{4′} is hydroxyl;
(c) each of R^{3′} and R^{5′} is bromo and each of R^{4′} and R^{6′} is hydroxyl; or
(d) each of R^{3′} and R^{5′} is bromo, R^{4′} is hydroxy, and R^{6′} is hydrogen.

20. The compound of claim 9 wherein each of R³, R^{3′}, R⁵ and R^{5′} is halo and each of R⁴, R^{4′}, R⁶, and R^{6′} is hydroxy.

21. The compound of claim 20 wherein each of R² and R^{2′} is hydrogen.

22. The compound of claim 9 wherein
(a) each of R³, R^{3′}, R⁵, and R^{5′} is halo and each of R⁴ and R^{4′} is hydroxyl;
(b) each of R³, R^{3′}, R⁵, and R^{5′} is bromo, and each of R⁴, R^{4′}, R⁶, and R^{6′} is hydroxyl; or
(c) each of R³, R^{3′}, R⁵, and R^{5′} is bromo, each of R⁴ and R^{4′} is hydroxy, and each of R⁶ and R^{6′} is hydrogen.

23. The compound of claim 22 wherein each of R² and R^{2′} is hydrogen.

24. The compound of either claim 1 or claim 2 wherein X and X′ are each the same or different and independently —NH—, —O—, or —S—.

25. The compound of claim 2 wherein the spacer J and spacer J′ are each 4,4′-diisothiocyanostilbene-2,2′-disulfonic acid (DIDS) and the compound has the following structure I(b): 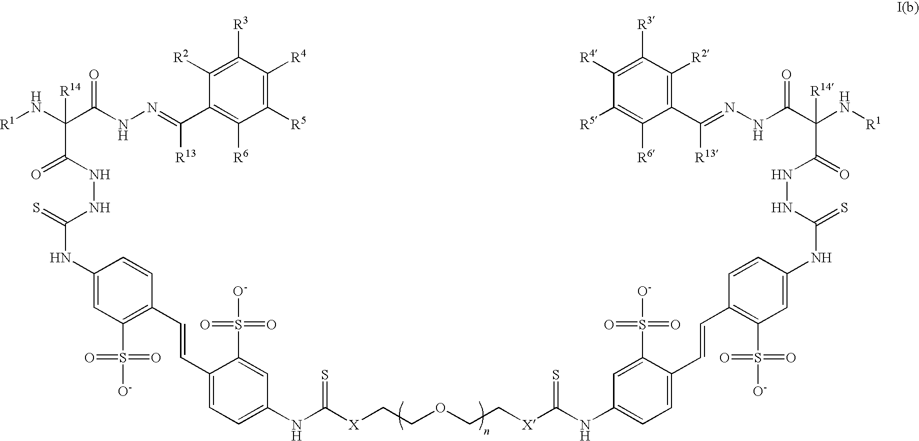
or a pharmaceutically acceptable salt, prodrug, or stereoisomer thereof, wherein:
R¹ and R^{1′} are the same or different and independently optionally substituted phenyl, optionally substituted heteroaryl, optionally substituted quinolinyl, optionally substituted anthracenyl, or optionally substituted naphthalenyl;
R², R^{2′}, R³, R^{3′}, R⁴, R^{4′}, R⁵, R^{5′}, R⁶, and R^{6′} are the same or different and independently hydrogen, hydroxy, C₁₋₈ alkyl, C₁₋₈ alkoxy, carboxy, halo, nitro, cyano, —SO₃H, —S(═O)₂NH₂, aryl, and heteroaryl;
R¹³, R^{13′}, R¹⁴, and R^{14′} are the same or different and independently hydrogen or C₁₋₈ alkyl;
X and X′ are each the same or different linker moiety; and
n is an integer between 0 and 2,500.

26. The compound of claim 25 wherein the compound is a sodium salt.

27. The compound of claim 25 wherein R¹³, R^{13′}, R¹⁴, and R^{14′} are the same or different and independently hydrogen or methyl.

28. The compound of claim 25 wherein R¹ and R^{1′} are the same or different and independently 1-naphthalenyl or 2-naphthalenyl, optionally substituted with one or more of halo, hydroxy, —SH, —SO₃H, C₁₋₈ alkyl, and C₁₋₈ alkoxy; aryloxy; phenyl substituted with one or more of hydroxy, C₁₋₈ alkyl, C₁₋₈ alkoxy, carboxy, —SO₃H, aryl, aryloxy, or halo; mono-halophenyl; di-halophenyl; mono-alkylphenyl; 2-anthracenyl; or 6-quinolinyl.

29. The compound of claim 28 wherein R¹ and R^{1′} are the same or different and independently 2-naphthalenyl, 2-chlorophenyl, 4-chlorophenyl, -2-4-dichlorophenyl, or 4-methylphenyl.

30. The compound of claim 28 wherein R¹ and R^{1′} are the same or different and independently mono-(halo)naphthalenyl; di-(halo)naphthalenyl; tri-(halo)naphthalenyl; mono-(hydroxy)naphthalenyl; di-(hydroxy)naphthalenyl; tri-(hydroxy)naphthalenyl; mono-(alkoxy)naphthalenyl; di-(alkoxy)naphthalenyl; tri-(alkoxy) naphthalenyl; mono-(aryloxy) naphthalenyl; di-(aryloxy)naphthalenyl; mono-(alkyl)naphthalenyl; di-(alkyl)naphthalenyl; tri-(alkyl)naphthalenyl; mono-(hydroxy)-naphthalene-sulfonic acid; mono-(hydroxy)-naphthalene-disulfonic acid; mono(halo)-mono (hydroxy)naphthalenyl; di(halo)-mono(hydroxy)naphthalenyl; mono(halo)-di(hydroxy)naphthalenyl; di(halo)-di(hydroxy)naphthalenyl; mono-(alkyl)-mono-(alkoxy)-naphthalenyl; or mono-(alkyl)-di-(alkoxy)-naphthalenyl.

31. The compound of claim 28 wherein R¹ and R^{1′} are the same or different and independently mono-(halo)phenyl; di-(halo)phenyl; tri-(halo)phenyl; mono-(hydroxy)phenyl; di-(hydroxy)phenyl tri-(hydroxy)phenyl; mono-(alkoxy)phenyl; di-(alkoxy)phenyl; tri-(alkoxy)phenyl; mono-(aryloxy)phenyl; di-(aryloxy)phenyl; mono-(alkyl)phenyl; di-(alkyl)phenyl; tri-(alkyl)phenyl; mono-(hydroxy)-phenyl-sulfonic acid; mono-(hydroxy)-phenyl-disulfonic acid; mono(halo)-mono(hydroxy)phenyl; di(halo)-mono (hydroxy)phenyl; mono(halo)-di(hydroxy)phenyl; di(halo)-di(hydroxy)phenyl; mono-(alkyl)-mono-(alkoxy)-phenyl; or mono-(alkyl)-di-(alkoxy)-phenyl.

32. The compound of claim 25 wherein R², R^{2′}, R³, R^{3′}, R⁴, R^{4′}, R⁵, R^{5′}, R⁶, and R^{6′} are the same or different and independently hydrogen, hydroxy, halo, C₁₋₈ alkyl, C₁₋₈ alkoxy, or carboxy.

33. The compound of claim 32 wherein R², R³, R⁴, R⁵, and R⁶ are each the same or different and independently selected such that the phenyl group to which R², R³, R⁴, R⁵, and R⁶ are attached is substituted with one, two, or three halo; one or two carboxy; one, two, or three hydroxy; one or two halo and one, two, or three hydroxy; one or two halo, one or two hydroxy, and one C₁₋₈ alkoxy; one or two halo, one hydroxy, and one or two C₁₋₈ alkoxy; or one halo, one or two hydroxy, and one or two C₁₋₈ alkoxy, wherein halo is bromo, chloro, iodo, or fluoro.

34. The compound of claim 32 wherein R^{2′}, R^{3′}, R^{4′}, R^{5′}, and R^{6′} are each the same or different and independently selected such that the phenyl group to which R^{2′}, R^{3′}, R^{4′}, R^{5′}, and R^{6′} are attached is substituted with one, two, or three halo; one or two carboxy; one, two, or three hydroxy; one or two halo and one, two, or three hydroxy; one or two halo, one or two hydroxy, and one C₁₋₈ alkoxy; one or two halo, one hydroxy, and one or two C₁₋₈alkoxy; or one halo, one or two hydroxy, and one or two C₁₋₈ alkoxy, wherein halo is bromo, chloro, iodo, or fluoro.

35.-36. (canceled)

37. The compound of claim 33 wherein R², R³, R⁴, R⁵, and R⁶ are the same or different and independently selected such that the phenyl group to which R², R³, R⁴, R⁵, and R⁶ is attached is 2-, 3-, or 4-halophenyl; 3,5-dihalophenyl; 2-, 3-, or 4-hydroxyphenyl; 2,4-dihydroxyphenyl; 3,5-dihalo-2,4,6-trihydroxyphenyl, 3,5-dihalo-2,4-dihydroxyphenyl; 3,5-dihalo-4-hydroxyphenyl; 3-halo-4-hydroxyphenyl; 3,5-dihalo-2-hydroxy-4-methoxyphenyl; or 4-carboxyphenyl.

38. The compound of claim 34 wherein R^{2′}, R^{3′}, R^{4′}, R^{5′}, and R^{6′} are the same or different and independently selected such that the phenyl group to which R^{2′}, R^{3′}, R^{4′}, R^{5′}, and R^{6′} is attached is 2-, 3-, or 4-halophenyl; 3,5-dihalophenyl; 2-, 3-, or 4-hydroxyphenyl; 2,4-dihydroxyphenyl; 3,5-dihalo-2,4,6-trihydroxyphenyl, 3,5-dihalo-2,4-dihydroxyphenyl; 3,5-dihalo-4-hydroxyphenyl; 3-halo-4-hydroxyphenyl; 3,5-dihalo-2-hydroxy-4-methoxyphenyl; or 4-carboxyphenyl.

39. The compound of claim 32 wherein halo is bromo.

40. The compound of claim 32 wherein
(a) each of R³ and R⁵ is halo and each of R⁴ and R⁶ is hydroxyl;
(b) each of R³ and R⁵ is halo and R⁴ is hydroxyl;
(c) each of R³ and R⁵ is bromo and each of R⁴ and R⁶ is hydroxyl; or
(d) each of R³ and R⁵ is bromo, R⁴ is hydroxy, and R⁶ is hydrogen.

41. The compound of claim 32 wherein
(a) each of R^{3′} and R^{5′} is halo and each of R^{4′} and R^{6′} is hydroxy;
(b) each of R^{3′} and R^{5′} is halo and R^{4′} is hydroxyl;
(c) each of R^{3′} and R^{5′} is bromo and each of R^{4′} and R^{6′} is hydroxyl; or
(d) each of R^{3′} and R^{5′} is bromo, R^{4′} is hydroxy, and R^{6′} is hydrogen.

42. The compound of claim 32 wherein
(a) each of R³, R^{3′}, R⁵ and R^{5′} is halo and each of R⁴, R^{4′}, R⁶, and R^{6′} is hydroxyl;
(b) each of R³, R^{3′}, R⁵, and R^{5′} is halo and each of R⁴ and R^{4′} is hydroxyl;
(c) each of R³, R^{3′}, R⁵, and R^{5′} is bromo, and each of R⁴, R^{4′}, R⁶, and R^{6′} is hydroxyl; or
(d) each of R³, R^{3′}, R⁵, and R^{5′} is bromo, each of R⁴ and R^{4′} is hydroxy, and each of R⁶ and R^{6′} is hydrogen.

43. The compound of any one of claims 40-42 wherein R² and R^{2′} are each hydrogen.

44. The compound of claim 25 wherein X and X′ are each the same or different and independently —NH—, —O—, or —S—.

45. (canceled)

46. The compound of claim 25 wherein the compound has one of the following structures I(c), I(d), I(e), or I(f):

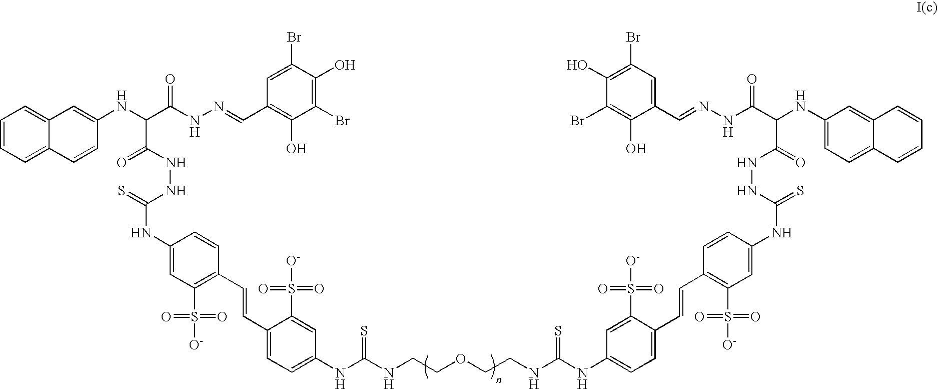

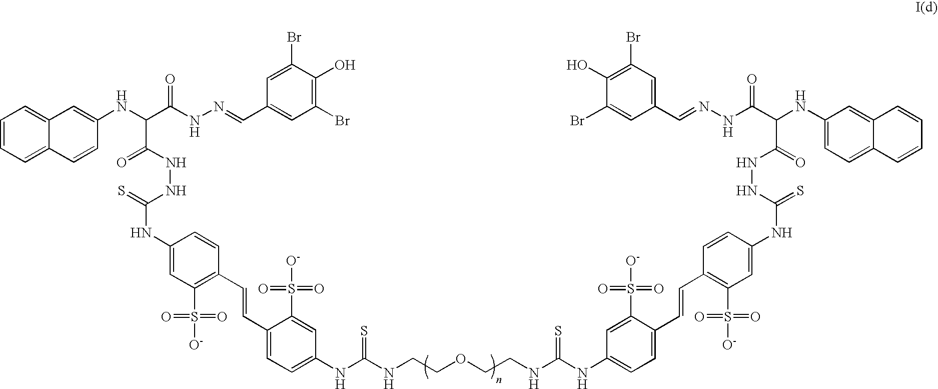

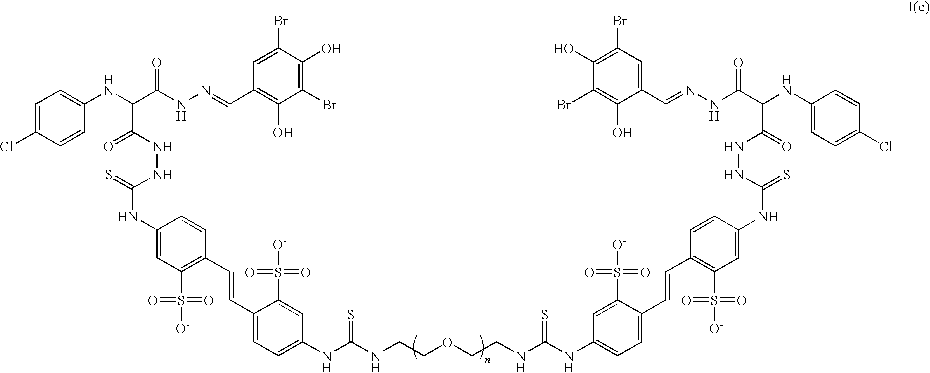

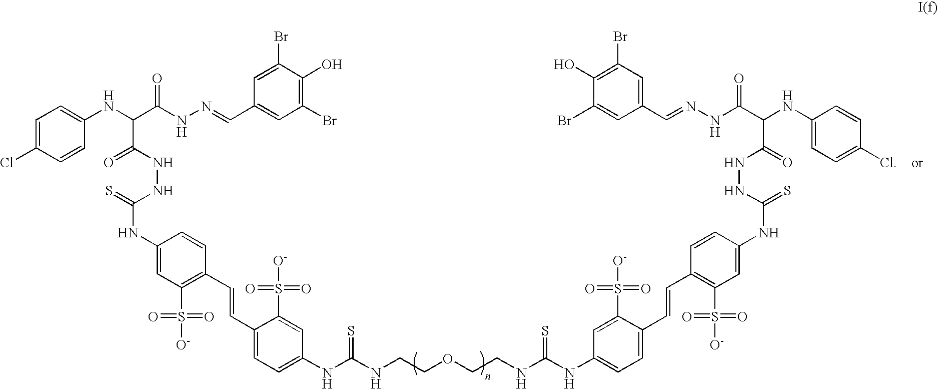

47. The compound of claim 46 wherein the compound is a sodium salt.

48. The compound of either claim 1 or claim 2 wherein n is an integer between 0 and 10, between 0 and 100, between 1 and 5, between 1 and 10, between 1 and 100, or between 1 and 1000.

49. The compound of either claim 1 or claim 2 wherein n is an integer between 50 and 1000, between 200-300, between 450 and 550, or between 900 and 1000.

50. A compound having the following structure II: 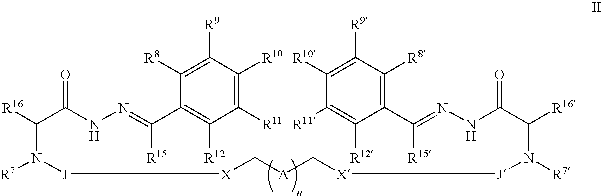
or a pharmaceutically acceptable salt, prodrug, or stereoisomer thereof
wherein:
R⁷ and R^{7′} are the same or different and independently optionally substituted phenyl, optionally substituted heteroaryl, optionally substituted quinolinyl, optionally substituted anthracenyl, or optionally substituted naphthalenyl;
R⁸, R^{8′}, R⁹, R^{9′}, R¹⁰, R^{10′}, R¹¹, R^{11′}, R¹² and R^{12′} are the same or different and independently hydrogen, hydroxy, C₁₋₈ alkyl, C₁₋₈ alkoxy, carboxy, halo, nitro, cyano, —SO₃H, —S(═O)₂NH₂, aryl, and heteroaryl;
R¹⁵, R^{15′}, R¹⁶, and R^{16′} are the same or different and independently hydrogen or C₁₋₈ alkyl;
X and X′ are each the same or different linker moiety;
J and J′ are each the same or different spacer moiety;
A is a polymer subunit; and
n is an integer between 0 and 2,500.

51. The compound of claim 50 wherein A is —CH₂—O—CH₂— and the compound has the following structure II(a): 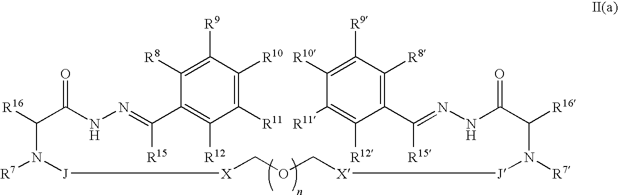
or a pharmaceutically acceptable salt, prodrug, or stereoisomer thereof
wherein:
R⁷ and R^{7′} are the same or different and independently optionally substituted phenyl, optionally substituted heteroaryl, optionally substituted quinolinyl, optionally substituted anthracenyl, or optionally substituted naphthalenyl;
R⁸, R^{8′}, R⁹, R^{9′}, R¹⁰, R^{10′}, R¹¹, R^{11′}, R¹² and R^{12′} are the same or different and independently hydrogen, hydroxy, C₁₋₈ alkyl, C₁₋₈ alkoxy, carboxy, halo, nitro, cyano, —SO₃H, —S(═O)₂NH₂, aryl, and heteroaryl;
R¹⁵, R^{15′}, R¹⁶, and R^{16′} are the same or different and independently hydrogen or C₁₋₈ alkyl;
X and X′ are each the same or different linker moiety;
J and J′ are each the same or different spacer moiety; and
n is an integer between 0 and 2,500.

52. The compound of claim 51 wherein spacer J and spacer J′ are each 4,4′-diisothiocyanostilbene-2,2′-disulfonic acid (DIDS) and the compound has the following structure II(b):

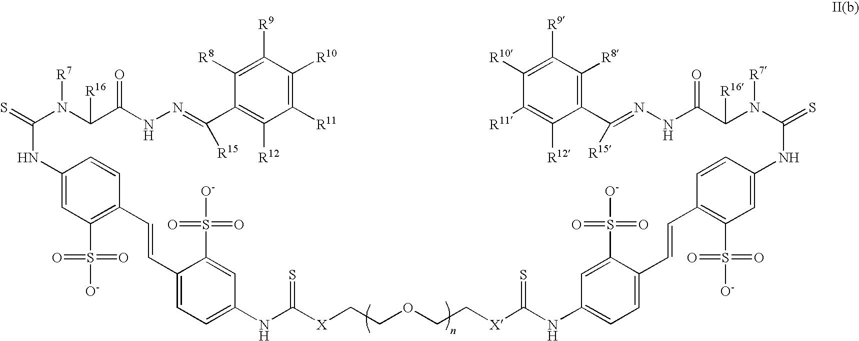

53. The compound of claim 52 wherein R¹⁵, R^{15′}, R¹⁶, and R^{16′} are the same or different and independently hydrogen or methyl.

54. The compound of claim 52 wherein R⁷ and R^{7′} are the same or different and independently unsubstituted phenyl, or substituted phenyl wherein phenyl is substituted with one or more of hydroxy, C₁₋₈ alkyl, C₁₋₈ alkoxy, carboxy, —SO₃H, aryl, aryloxy, or halo.

55. The compound of claim 54 wherein R⁷ and R^{7′} are the same or different and independently mono-(halo)phenyl; di-(halo)phenyl; tri-(halo)phenyl; mono-(hydroxy)phenyl; di-(hydroxy)phenyl tri-(hydroxy)phenyl; mono-(alkoxy)phenyl; di-(alkoxy)phenyl; tri-(alkoxy)phenyl; mono-(aryloxy)phenyl; di-(aryloxy)phenyl; mono-(alkyl)phenyl; di-(alkyl)phenyl; tri-(alkyl)phenyl; mono-(hydroxy)-phenyl-sulfonic acid; mono-(hydroxy)-phenyl-disulfonic acid; mono(halo)-mono(hydroxy)phenyl; di(halo)-mono (hydroxy)phenyl; mono(halo)-di(hydroxy)phenyl; di(halo)-di(hydroxy)phenyl; mono-(alkyl)-mono-(alkoxy)-phenyl; or mono-(alkyl)-di-(alkoxy)-phenyl.

56. The compound of claim 55 wherein halo is chloro.

57. The compound of claim 54 wherein R⁷ and R^{7′} are the same or different and independently substituted phenyl wherein phenyl is substituted with methyl or chloro.

58. The compound of 52 wherein R⁷ and R^{7′} are the same or different and independently quinolinyl or anthracenyl, optionally substituted with one or more of halo, hydroxy, C₁₋₈ alkyl, or C₁₋₈ alkoxy.

59. The compound of claim 52 wherein R⁷ and R^{7′} are the same or different and independently 2-naphthalenyl or 1-naphthalenyl, optionally substituted with one or more of halo, hydroxy, —SH, —SO₃H, C₁₋₈ alkyl, aryl, aryloxy, or C₁₋₈ alkoxy.

60. The compound of claim 59 wherein R⁷ and R^{7′} are the same or different and independently mono-(halo)naphthalenyl; di-(halo)naphthalenyl; tri-(halo)naphthalenyl; mono-(hydroxy)naphthalenyl; di-(hydroxy)naphthalenyl; tri-(hydroxy)naphthalenyl; mono-(alkoxy)naphthalenyl; di-(alkoxy)naphthalenyl; tri-(alkoxy)naphthalenyl; mono-(aryloxy)naphthalenyl; di-(aryloxy)naphthalenyl; mono-(alkyl)naphthalenyl; di-(alkyl)naphthalenyl; tri-(alkyl)naphthalenyl; mono-(hydroxy)-naphthalene-sulfonic acid; mono-(hydroxy)-naphthalene-disulfonic acid; mono(halo)-mono(hydroxy)naphthalenyl; di(halo)-mono(hydroxy)naphthalenyl; mono(halo)-di(hydroxy)naphthalenyl; di(halo)-di(hydroxy)naphthalenyl; mono-(alkyl)-mono-(alkoxy)-naphthalenyl; or mono-(alkyl)-di-(alkoxy)-naphthalenyl.

61. The compound of claim 52 wherein R⁷ and R^{7′} are the same or different and independently 2-chlorophenyl, 4-chlorophenyl, 2,4-chlorophenyl, 4-methylphenyl, 2-anthracenyl, or 6-quinolinyl.

62. The compound of claim 52 wherein R⁷ and R^{7′} are the same or different and independently 2-naphthalenyl or 1-naphthalenyl.

63. The compound of claim 52 wherein R⁸, R⁹, R¹⁰, R¹¹, R¹², R^{8′}, R^{9′}, R^{10′}, R^{11′}, and R^{12′} are each the same or different and independently hydrogen, hydroxy, halo, C₁₋₈ alkyl, C₁₋₈ alkoxy, or carboxy.

64. The compound of any one of claims 63 wherein R⁸, R⁹, R¹⁰, R¹¹, and R¹² are each the same or different and independently selected such that the phenyl group to which R⁸, R⁹, R¹⁰, R¹, and R¹² are attached is substituted with one, two, or three halo; one or two carboxy; one, two, or three hydroxy; one or two halo and one, two, or three hydroxy; one or two halo, one or two hydroxy, and one C₁₋₈ alkoxy; one or two halo, one hydroxy, and one or two C₁₋₈ alkoxy; or one halo, one or two hydroxy, and one or two C₁₋₈ alkoxy.

65. The compound of any one of claims 63 wherein R^{8′}, R^{9′}, R^{10′}, R^{11′}, and R^{12′} are each the same or different and independently selected such that the phenyl group to which R^{8′}, R^{9′}, R^{10′}, R^{11′}, and R^{12′} are attached is substituted with one, two, or three halo; one or two carboxy; one, two, or three hydroxy; one or two halo and one, two, or three hydroxy; one or two halo, one or two hydroxy, and one C₁₋₈ alkoxy; one or two halo, one hydroxy, and one or two C₁₋₈ alkoxy; or one halo, one or two hydroxy, and one or two C₁₋₈ alkoxy.

66.-67. (canceled)

68. The compound of claim 64 wherein R⁸, R⁹, R¹⁰, R¹¹, and R¹² are each the same or different and independently selected such that the phenyl group to which R⁸, R⁹, R¹⁰, R¹¹, and R¹² are attached is 2-, 3-, or 4-halophenyl; 3,5-dihalophenyl; 2-, 3-, or 4-hydroxyphenyl; 2,4-dihydroxyphenyl; 3,5-dihalo-2,4,6-trihydroxyphenyl; 3,5-dihalo-2,4-dihydroxyphenyl; 3,5-dihalo-4-hydroxyphenyl; 3-halo-4-hydroxyphenyl; 3,5-dihalo-2-hydroxy-4-methoxyphenyl; or 4-carboxyphenyl.

69. The compound of claim 65 wherein R^{8′}, R^{9′}, R^{10′}, R^{11′}, and R^{12′} are each the same or different and independently selected such that the phenyl group to which R^{8′}, R^{9′}, R^{10′}, R^{11′}, and R^{12′} are attached is 2-, 3-, or 4-halophenyl; 3,5-dihalophenyl; 2-, 3-, or 4-hydroxyphenyl; 2,4-dihydroxyphenyl; 3,5-dihalo-2,4,6-trihydroxyphenyl, 3,5-dihalo-2,4-dihydroxyphenyl; 3,5-dihalo-4-hydroxyphenyl; 3-halo-4-hydroxyphenyl; 3,5-dihalo-2-hydroxy-4-methoxyphenyl; or 4-carboxyphenyl.

70. The compound of claim 63 wherein halo is bromo.

71. The compound of claim 63 wherein
(a) each of R⁹ and R¹¹ is halo and each of R¹⁰ and R¹² is hydroxyl;
(b) each of R⁹ and R¹¹ is halo and R¹⁰ is hydroxyl;
(c) each of R⁹ and R¹¹ is bromo, and each of R¹⁰ and R¹² is hydroxy; or
(d) each of R⁹ and R^{11′} is bromo, R¹⁰ is hydroxy, and R¹² is hydrogen.

72. The compound of claim 63 wherein
(a) each of R^{9′} and R^{11′} is halo and each of R^{10′} and R^{12′} is hydroxyl;
(b) each of R^{9′} and R^{11′} is halo and R^{10′} is hydroxyl;
(c) each of R^{9′} and R^{11′} is bromo, and each of R^{10′} and R^{12′} is hydroxyl; or
(d) each of R^{9′} and R^{11′} is bromo, R^{10′} is hydroxy, and R^{12′} is hydrogen.

73. The compound of claim 63 wherein
(a) each of R⁹, R^{9′}, R¹¹ and R^{11′} is halo and each of R¹⁰, R^{10′}, R¹², and R^{12′} is hydroxyl;
(b) each of R⁹, R^{9′}, R¹¹, and R^{11′} is halo and each of R¹⁰ and R^{10′} is hydroxyl;
(c) each of R⁹, R^{9′}, R¹¹, and R^{11′} is bromo, and each of R¹⁰, R^{10′}, R¹², and R^{12′} is hydroxyl; or
(d) each of R⁹, R^{9′}, R¹¹, and R^{11′} is bromo, each of R¹⁰ and R^{10′} is hydroxy, and each of R¹² and R^{12′} is hydrogen.

74. The compound of claim 73 wherein R⁸ and R^{8′} are each hydrogen.

75. The compound of claim 52 wherein X and X′ are each the same or different and independently —NH—, —O—, or —S—.

76. (canceled)

77. The compound of claim 52 wherein the compound is a sodium salt.

78. The compound of claim 52 wherein the compound has one of the following structures II(c), II(d), II(e), or II(f): 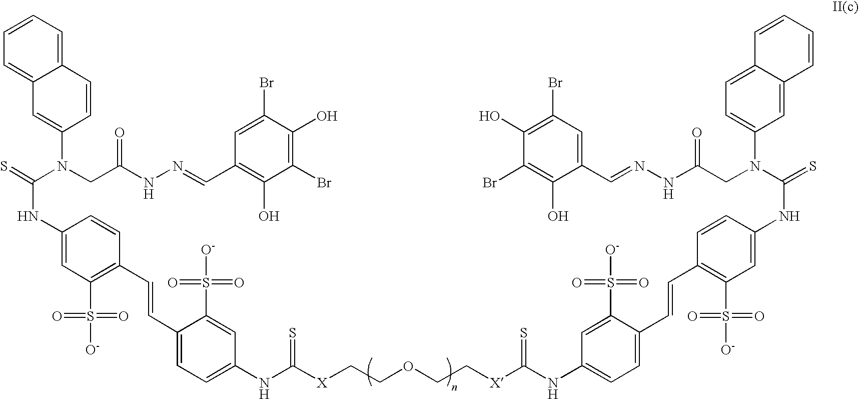 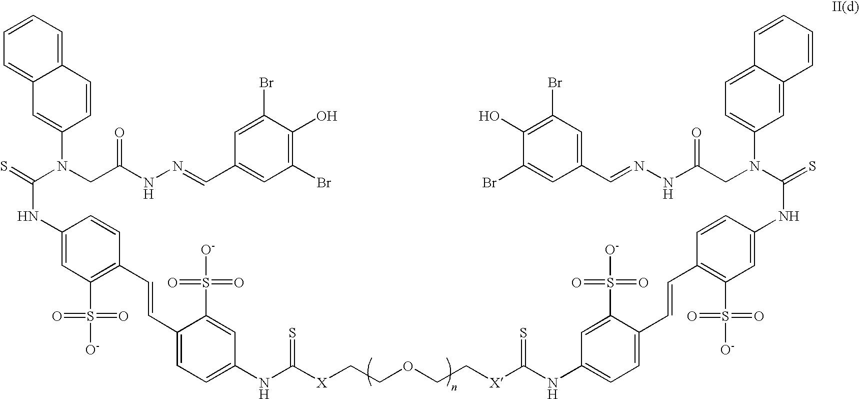 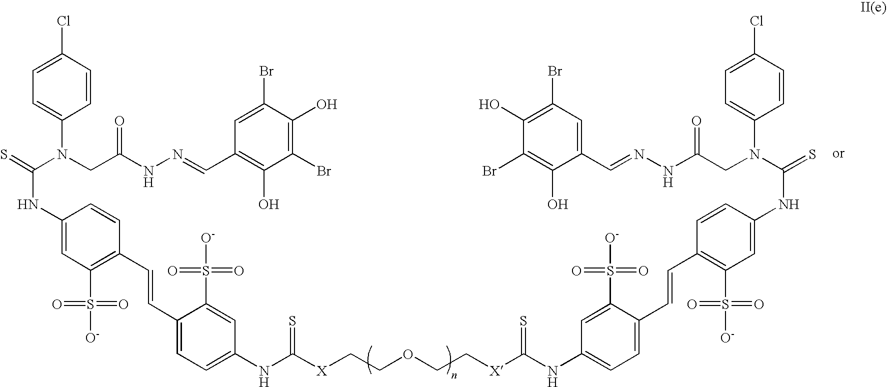 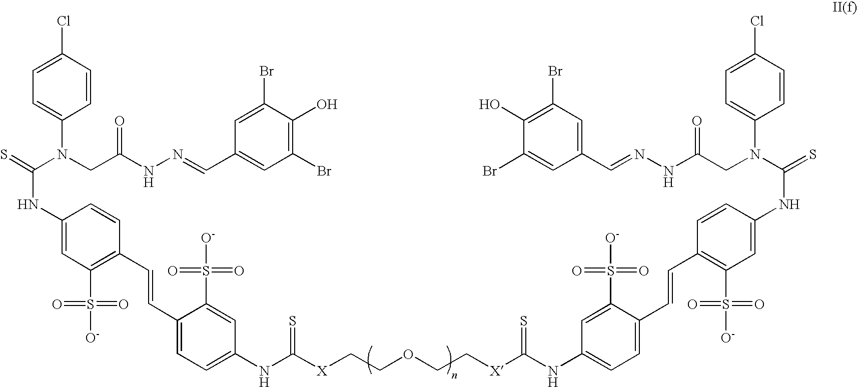
wherein X and X′ are each independently —NH—, —O—, or —S—.

79. The compound of claim 78 wherein each of X and X′ is —NH—.

80. The compound of claim 78 wherein the compound is a sodium salt.

81. The compound of claim 50 wherein n is an integer between 0 and 10, between 0 and 100, between 1 and 5, between 1 and 10, between 1 and 100, between 1 and 1000, or between 50 and 1000.

82. The compound of claim 81 wherein n is an integer between 200 and 300, between 450 and 550, or between 900 and 1000.

83. A composition comprising the compound of claim 1 or claim 50 and a pharmaceutically acceptable excipient.

84. A method of treating a disease or disorder associated with aberrantly increased ion transport by cystic fibrosis transmembrane conductance regulator (CFTR), the method comprising administering to a subject the composition according to claim 83, wherein ion transport by CFTR is inhibited.

85. The method according to claim 84 wherein the disease or disorder is selected from aberrantly increased intestinal fluid secretion and secretory diarrhea.

86.-92. (canceled)

93. A method of inhibiting ion transport by a cystic fibrosis transmembrane conductance regulator (CFTR) comprising contacting (a) a cell that comprises CFTR and (b) the compound of either claim 1 or claim 50, under conditions and for a time sufficient for the CFTR and the compound to interact, thereby inhibiting ion transport by CFTR.

94. A method of treating secretory diarrhea comprising administering to a subject a pharmaceutically acceptable excipient and the compound of either claim 1 or claim 50.

95. (canceled)

96. The compound of claim 31 wherein halo is chloro.
